(19) United States
(12) Patent Application Publication
(10) Pub. No.: US 2026/0108728 A1
(43) Pub. Date: Apr. 23, 2026

(54) SYSTEMS, DEVICES, AND METHODS FOR DIRECTING SHOCK WAVES USING A NOZZLE

(71) Applicant: Shockwave Medical, Inc., Santa Clara, CA (US)

(72) Inventors: Alyssa MCCULLOCH, Santa Clara, CA (US); Robert ZELENKA, Milpitas, CA (US); Daryl WONG, San Jose, CA (US); Thu Anh HO, San Jose, CA (US); Steven Yihlih PENG, Fremont, CA (US)

(73) Assignee: Shockwave Medical, Inc., Santa Clara, CA (US)

(21) Appl. No.: 19/362,998

(22) Filed: Oct. 20, 2025

Related U.S. Application Data
(60) Provisional application No. 63/710,534, filed on Oct. 22, 2024.

(51) Int. Cl. A61N 1/05; A61B 17/00; A61B 17/22; A61M 25/00

(52) U.S. Cl. CPC A61N 1/057; A61B 17/22022; A61B 2017/00292; A61B 2017/22008; A61B 2017/22024; A61B 2017/22025; A61B 2017/22079; A61M 25/0082; A61N 2001/0578

(57) ABSTRACT

A method of facilitating pacemaker lead removal using a shock wave catheter includes: advancing a catheter over a pacemaker lead, the catheter comprising a nozzle comprising an outlet sized to receive the pacemaker lead, and at least one shock wave emitter positioned proximally of the nozzle outlet; positioning the nozzle outlet adjacent to a treatment site comprising a lesion located at least partially distal to the nozzle outlet; generating one or more shock waves that propagate distally within the nozzle and are concentrated by the nozzle to disrupt the lesion to facilitate removal of the pacemaker lead.

### CROSS-REFERENCE TO RELATED APPLICATIONS

This application claims priority to U.S. Provisional Application No. 63/710,534, filed Oct. 22, 2024, the content of which is incorporated herein by reference in its entirety.

### FIELD

The present disclosure relates generally to the field of medical devices and methods, and more specifically to shock wave catheter devices for treating calcified lesions in body lumens, such as calcified lesions and occlusions in vasculature and kidney stones in the urinary system.

### BACKGROUND

The accumulation of calcium in a patient's blood vessels, tissues, or other organs can cause calcification that may disrupt organ function and lead to health issues for the patient. For example, when vascular plaque builds up along and in the walls of the coronary arteries, the accumulation can narrow the passageway of the vessel (referred to as stenosis) and restrict blood flow to the heart muscle, which can eventually lead to a heart attack. Treating stenosis is even more challenging when the plaque becomes hardened due to calcification.

A wide variety of catheters have been developed for treating stenotic blood vessels that are narrowed by the progressive growth and accumulation of plaque, a condition also known as atherosclerosis. For example, treatment systems for percutaneous coronary angioplasty or peripheral angioplasty use angioplasty balloons to dilate a calcified lesion and restore normal blood flow in a vessel. In these types of procedures, a catheter carrying a balloon is advanced into the vasculature along a guide wire until the balloon is aligned with calcified plaques. The balloon is then pressurized (normally to greater than 10 atm), causing the balloon to expand in a vessel to push calcified plaques back into the vessel wall and dilate occluded regions of vasculature. A particular focus is to treat calcified lesions of plaque, such as calcified lesions in the vasculature associated with arterial disease. When treating calcified lesions, it is important to minimize damage to surrounding soft tissues while still breaking up the lesion as much as possible.

However, traditional dilation balloon angioplasty therapies may not work with calcified tissue because the calcium in the atherosclerotic plaque hardens the lesion, resisting the mechanical force of balloon expansion. The resistance can result in more procedural complication and vessel damage because the high-pressure balloons preferentially expand away from the hard calcified tissue. The predisposition of the ballon to expand in a direction of lower resistance increases the risk of major dissection or perforation of the vessel, often at the ends of a lesion at the interface between healthy tissue and calcified tissue (i.e., where the balloon encounters soft tissue). In the case of an eccentric calcified lesion where the hardened region is biased on a side of a vessel, the expansion ends up going preferentially in the direction opposite of the calcified region of the lesion, straining and dissecting the healthier side of the blood vessel. Moreover, in the case of nodular calcium, expansion of a standard angioplasty balloon can lead to pushing the node of calcified material in a manner that may puncture the vessel.

Another approach to dealing with calcified stenotic plaque is to cut away at a calcified lesion, by using a cutting or scoring balloon, an angioplasty balloon having a raised structure on the surface of the balloon (e.g., an angioplasty balloon with blade-like structures on its exterior). The expansion of an angioplasty balloon having a raised structure may allow for mechanical force on a lesion to be focused at the location of the raised structure, but these devices still do not provide for any protection from dissection or perforation resulting from preferential expansion of the balloon away from hardened tissue. Another technique for cutting away at a calcified lesion is by using an atherectomy device, which typically includes a motor-driven rotating or oscillating blade that is pushed into and cuts through an occlusion (also referred to as “debulking” or “extirpation”). Because these treatments work by liberating the calcified tissues from a blood vessel wall, there is an increased risk of embolism where the free-floating masses of calcification may proceed down the blood stream. Such systems may include baskets to capture or negative-pressure lumens to aspirate such unmoored emboli as a necessary additional structure to ensure the safety of such devices. An additional concern for atherectomy devices is that the movement or rotation of atherectomy catheter blades generates frictional heat and can cause a related thermal injury from mere operation of the atherectomy device. That heat can directly injure the lining of a blood vessel and can also lead to an increased risk of blood clotting. Naturally, the action of a moving blade within the vasculature also significantly increases the potential for a large dissection and perforation of the blood vessel by the blade itself.

Accordingly, there is an ongoing need for improved medical devices and treatments to address calcification and restore organ function. One such treatment is intravascular lithotripsy (IVL), which uses acoustic pressure to break up the calcified regions. In IVL, a device such as a catheter is advanced within the patient's body to a position adjacent to the treatment area. The IVL device is configured to generate acoustic waves, specifically, ultrasonic short pulse waves (also known as “shock waves”), which propagate outward from the IVL device to modify the calcified regions. The acoustic pressure of the shock waves may crack and disrupt the calcified regions near the IVL device without harming the surrounding blood vessels, tissues, or other organs. In particular, IVL can address and treat calcified plaques and stenosis with a safety profile that minimizes risk of blood vessel damage and with an efficacy profile that provides for durable circulatory restoration.

Calcium buildup in various body structures, such as mitral annular calcification (MAC) and chronic total occlusions (CTOs) or fibrotic tissue buildup surrounding pacemaker leads in cardiac tissue, can become very thick and thus difficult to treat. Such lesions are often difficult to treat using radially-firing shock wave generating devices, which typically include shock wave emitters spaced along the length (i.e., along the longitudinal axis) of the device's body and are used to treat buildup of calcified plaque along the length of the inner wall of a body lumen such as a blood vessel. Such devices are not configured for generating shock waves primarily directed forward of the catheter.

### SUMMARY

Described herein are shock wave generating systems, devices, and methods that utilize a nozzle to direct and concentrate shock waves and/or cavitation bubbles in a forward (i.e., distal) direction for treating a treatment area located distally of the nozzle. The nozzle may converge from a larger inlet portion to a narrower outlet portion. Shock waves are concentrated as they propagate toward the nozzle outlet portion. Concentration of the shock waves and/or cavitation bubbles may result in a more targeted sonic output and corresponding increase in force exerted on a target treatment area. As shock waves concentrate within the nozzle as they move toward an outlet, fluid within the nozzle is pushed forward toward the nozzle outlet, increasing in and velocity at the outlet of the nozzle. The fluid is ejected from the nozzle outlet and projects distally of the nozzle outlet toward a target treatment area.

Also disclosed are devices and methods that provide directional control of shock-wave propagation. A deflector (e.g., an acoustically reflective wall) positioned distal to one or more shock wave emitters may be positioned to intercept energy propagating distally from the one or more shock wave emitters and redirect at least a portion of that energy radially toward a target site. During operation, shock waves (and, optionally, fluid and/or cavitation bubbles) generated by the emitter(s) propagate distally toward the deflector, and the deflector redirects the shock waves at least partially radially outward toward a lesion or other treatment site. This radial re-direction of the shock waves may enable enhanced range of treatment via simple rotation of the catheter within a vessel or other body lumen.

An exemplary catheter includes a catheter body and at least one shock wave emitter disposed at a distal end of the catheter body that is configured to generate shock waves that propagate in a distal direction relative to the catheter body. A nozzle is disposed at the distal end of the catheter such that shock waves generated by the emitter propagate within the nozzle toward an outlet of the nozzle, which may cause the shock waves to concentrate together at the outlet of the nozzle. The catheter can be used by positioning the catheter such that occlusive material to be treated is at least partially within the outlet of the nozzle and/or positioned distally of the nozzle. One or more shock waves may then be generated such that shock waves and/or cavitation bubbles are concentrated at the nozzle outlet, impinging on occlusive material within the nozzle outlet and/or directed distally following concentration at the nozzle outlet toward the occlusive material.

According to an aspect, a catheter for use in a body lumen includes: a catheter body; at least one shock wave emitter disposed at a distal end of the catheter body and configured to generate at least one shock wave that propagates distally of the catheter body; and a nozzle disposed at a distal end of the catheter body and configured to direct the at least one shock wave to an outlet of the nozzle.

Optionally, the nozzle is configured to receive a pacemaker lead. Optionally, the catheter includes a central lumen extending along the length of the catheter body to an inlet of the nozzle, wherein the central lumen is configured to receive the pacemaker lead. Optionally, the distal end of the nozzle includes a beveled edge for dislodging fibrotic tissue from the pacemaker lead. Optionally, the nozzle is configured to concentrate the at least one shock wave generated by the at least one shock wave emitter at the outlet of the nozzle. Optionally, the nozzle is configured to direct at least one bubble resulting from the at least one shock wave to the outlet of the nozzle. Optionally, the nozzle is configured to concentrate the at least one bubble at the outlet of the nozzle. Optionally, the nozzle includes stainless-steel, high-density polyethylene, polyvinyl chloride, or a combination thereof. Optionally, an inlet of the nozzle is positioned distally of the at least one shock wave emitter such that the at least one shock wave enters the nozzle through the inlet. Optionally, the outlet of the nozzle forms a distal end of the catheter. Optionally, an inlet of the nozzle overlaps with at least a portion of the at least one shock wave emitter with respect to a longitudinal axis of the catheter body. Optionally, the nozzle is a convergent nozzle. Optionally, the at least one shock wave emitter is positioned radially inward of an outer diameter of an inlet to the nozzle.

Optionally, the catheter includes a fluid supply line extending to an inlet of the nozzle. Optionally, the fluid supply line is configured to supply a fluid to an inlet of the nozzle to replace fluid that exits the outlet of the nozzle when the respective shock waves are generated by the one or more shock wave emitters. Optionally, the catheter includes a fluid return line configured to remove debris from the nozzle. Optionally, the nozzle is removably attached to the catheter body. Optionally, the at least one shock wave emitter includes an exposed end of a first insulated wire separated by a spark gap from an exposed end of a second insulated wire.

Optionally, the at least one shock wave emitter includes at least one electrical connection to an electrode of at least one other shock wave emitter. Optionally, the at least one shock wave emitter includes a first electrode and a conductive emitter band that is separated from the first electrode by a spark gap.

Optionally, the catheter body includes: a cavity at the distal end of the catheter body; at least one radially firing shock wave emitter positioned outwardly of the cavity and configured to generate at least one shock wave of the at least one shock wave; and a shield surrounding the catheter body and covering the at least one radially firing shock wave emitter such that shock waves generated by the at least one radially firing shock wave emitter are reflected by the shield into the cavity at the distal end of the catheter body and directed to the outlet of the nozzle.

According to an aspect, a method for removing a pacemaker lead wire includes: advancing the catheter of any of the examples described herein along the pacemaker lead to a target site comprising fibrotic tissue; and generating one or more shock waves to at least partially break up the fibrotic tissue so that the pacemaker lead can be removed. Optionally, the target site is within the heart. Optionally, the target site is located distally of the distal end of the catheter body.

According to an aspect, a shock wave generating system includes: a shock wave energy generator; and the catheter of any of the examples described herein. Optionally, the shock wave energy generator is configured to deliver voltage pulses to a shock wave emitter of the plurality of shock wave emitters. Optionally, the shock wave energy generator is configured to deliver the voltage pulses at a frequency of at least 1 Hz.

According to an aspect, a method for treating an occlusion in a body lumen with shock waves includes positioning a distal portion of a catheter adjacent to the occlusion in the body lumen; emitting one or more shock waves from one or more shock wave emitters located at the distal portion of the catheter such that the shock waves propagate in a distal direction; and directing the shock waves by a nozzle located at a distal end of the catheter to an outlet of the nozzle for treating the occlusion. Optionally, directing the one or more shock waves to the outlet of the nozzle includes concentrating the one or more shock waves. Optionally, the method includes directing, by the nozzle, at least one bubble generated by the one or more shock wave emitters toward an outlet of the nozzle, wherein directing the at least one bubble toward the outlet of the nozzle causes the at least one bubble to concentrate at the outlet and propagate distally of the outlet.

Optionally, the method includes advancing the catheter further into the body lumen; and emitting a one or more additional shock waves from the one or more shock wave emitters so that the shock waves concentrate at the outlet of the nozzle and propagate distally of the catheter body via the outlet of the nozzle. Optionally, the method includes supplying fluid via a nozzle inlet to replace fluid that exits the outlet of the nozzle when emitting the one or more shock waves. Optionally, the method includes removing debris from the body lumen via the outlet of the nozzle using a fluid return line of the catheter that extends from an inlet of the nozzle along the length of the catheter body.

According to an aspect, a method of facilitating pacemaker lead removal using a shock wave catheter includes: advancing a catheter over a pacemaker lead, the catheter comprising a nozzle comprising an outlet sized to receive the pacemaker lead, and at least one shock wave emitter positioned proximally of the nozzle outlet; positioning the nozzle outlet adjacent to a treatment site comprising a lesion located at least partially distal to the nozzle outlet; generating one or more shock waves that propagate distally within the nozzle and are concentrated by the nozzle to disrupt the lesion to facilitate removal of the pacemaker lead.

The treatment site may be within the heart. The nozzle may be a convergent nozzle configured to concentrate the one or more shock waves at or beyond the nozzle outlet. The lesion may be disposed at least partially on the pacemaker lead such that as the catheter is advanced over the pacemaker lead, the lesion is received into the nozzle outlet. The shock waves concentrated at the nozzle outlet may impinge on the lesion within the nozzle and at the outlet. The nozzle may be configured such that a substantially uniform pressure is applied to the pacemaker lead by the one or more shock waves.

The nozzle outlet may include a beveled edge or a tapered edge. The method may include advancing the catheter distally toward the lesion and scraping the lesion from the pacemaker lead using the beveled edge. The one or more shock waves may impinge upon the lesion distally of the nozzle outlet. The method may include supplying a fluid to an inlet of the nozzle while the one or more shock waves are generated. The method may include directing, by the nozzle, a plurality of bubbles generated by the at least one shock wave emitter toward an outlet of the nozzle, wherein the nozzle concentrates the bubbles at the outlet. The method may include aspirating debris from a body lumen in which the pacemaker lead is positioned via the outlet of the nozzle. The nozzle may be formed from an acoustically reflective material. The nozzle may be configured to concentrate the one or more shock waves at a longitudinal axis of the catheter. The nozzle may be configured to concentrate the one or more shock waves at an position offset from a longitudinal axis of the catheter. The at least one shock wave emitter may include a plurality of shock wave emitters arrayed symmetrically about a longitudinal axis of the catheter. The at least one shock wave emitter may be formed by a distal end of a first wire and a distal end of a second wire separated by a spark gap.

According to an aspect, a catheter for treating a lesion in a body lumen may include: a catheter body; at least one shock wave emitter disposed at a distal end of the catheter body and configured to generate at least one shock wave; a cap positioned at least partially distally of the at least one shock wave emitter, the distal cap comprising: a closed distal end; an outer wall extending between the catheter body and the distal end, the outer wall comprising an aperture; a deflector positioned between the distal end and the at least one shock wave emitter, the deflector oriented at an oblique angle relative to a longitudinal axis of the catheter body and configured to direct shock waves generated using the at least one shock wave emitter outward through the aperture. A distal edge of the deflector may be longitudinally aligned with a distal edge of the aperture. A proximal edge of the deflector may be longitudinally aligned with a proximal edge of the aperture. The deflector may be formed from acoustically reflective material. The distal cap may include at least one radiopaque marker aligned with an edge of the aperture.

### BRIEF DESCRIPTION OF THE FIGURES

The invention will now be described, by way of example only, with reference to the accompanying drawings, in which:

FIG. 1 illustrates a system for treating calcifications in body lumens according to some examples.

FIG. 2 illustrates an exemplary catheter including a nozzle and a plurality of shock wave emitters according to some examples.

FIGS. 3A and 3B illustrates exemplary bubble formation, propagation, and collapse with and without a nozzle, according to some examples.

FIGS. 4A and 4B illustrate exemplary catheters various nozzle configurations according to some examples.

FIG. 5 illustrates an exemplary catheter including a nozzle for treating calcifications and fibrotic tissue around a pacemaker lead according to some examples.

FIG. 6 illustrates an exemplary method for generating shock waves concentrated using a nozzle to break up tissue around a pacemaker lead according to some examples.

FIG. 7 illustrates an exemplary method for generating shock waves concentrated within a nozzle according to some examples.

FIG. 8A illustrates an exemplary catheter including a nozzle and a plurality of shock wave emitters according to some examples.

FIG. 8B illustrates a detailed view of a shock wave emitter according to some examples.

FIG. 8C illustrates a side view of an exemplary catheter including a nozzle and a plurality of shock wave emitters according to some examples.

FIGS. 9A-9D illustrate various configurations of shock wave emitters on a distal end of a catheter including a nozzle according to some examples.

FIGS. 10A-10B illustrate an exemplary catheter including a nozzle and a plurality of shock wave emitters that include a conductive emitter band according to some examples.

FIG. 11 illustrates an exemplary catheter including a nozzle and a plurality of shock wave emitters that include a conductive emitter band according to some examples.

FIG. 12A illustrates a front view of a catheter including shock waves that share a common electrode and a nozzle positioned at the distal end of the catheter according to some examples.

FIG. 12B illustrates a side view of the catheter of FIG. 12A.

FIGS. 13A-13B illustrate an exemplary catheter including a nozzle and at least one shock wave emitter according to some examples.

FIGS. 14A-14C illustrate cross-section views of exemplary catheters including at least one shock wave emitter and a nozzle according to some examples.

FIGS. 15A-15E illustrate exemplary slotted emitter sheaths according to some examples.

FIGS. 16A-16C illustrate exemplary shock wave emitters including slotted emitter sheaths according to some examples.

FIGS. 17A-17B illustrate exemplary slotted emitter sheaths according to some examples.

FIGS. 18A-18B illustrate an exemplary catheter including a conventional emitter sheath according to some examples.

FIG. 18C illustrates the distal end of an exemplary catheter with a shock wave generator having a slotted emitter and a nozzle according to some examples.

FIG. 18D illustrates a cross sectional view of the catheter of FIG. 18C, according to some examples.

FIG. 18E illustrates the distal end of an exemplary catheter with a shock wave generator having a slotted emitter and a nozzle according to some examples.

FIGS. 19A-19D illustrate an exemplary nozzle according to some examples.

FIGS. 20A-20B illustrate aspects of an exemplary catheter with a distal cap configured to deflect shock waves according to some examples.

FIG. 21 illustrates aspects of an exemplary method of facilitating pacemaker lead removal using a shock wave catheter

FIG. 22 illustrates aspects of an exemplary computing system according to some examples.

### DETAILED DESCRIPTION

The following description is presented to enable a person of ordinary skill in the art to make and use the various embodiments and aspects thereof disclosed herein. Descriptions of specific catheters, systems, methods, and applications are provided only as examples. Various modifications to the examples described herein will be readily apparent to those of ordinary skill in the art, and the general principles described herein may be applied to other examples and applications without departing from the spirit and scope of the various embodiments and aspects thereof. Thus, the various embodiments and aspects thereof are not intended to be limited to the examples described herein and shown but are to be accorded the scope consistent with the claims.

In recent years, in order to treat atherosclerosis and related conditions, the technique and treatment of intravascular lithotripsy (“IVL”) has been developed. IVL is an interventional procedure that modifies calcified plaque in diseased vasculature. More precisely, IVL is the energy-based generation of ultrasonic acoustic pressure waves (also known as ultrasonic short pressure pulses) for modification, fracture, and/or fragmentation of calcified plaque in situ. The ultrasonic acoustic pressure waves are created by rapid energy absorption in a fluid-filled cavity. These ultrasonic acoustic pressure waves subsequently propagate to impact the calcium in the vessel walls thereby improving arterial compliance and enabling optimal lumen expansion in vascular intervention procedures. The mechanism of plaque modification is through use of a catheter having one or more ultrasonic short pressure pulses (commonly referred to as “shock waves”) emit from a generating source located within a liquid, often via plasma generation, with the generating source configured to create ultrasonic short pressure pulses that modify and fragment the calcified plaque. While IVL is most commonly associated with the fragmentation of vascular calcifications, it is appreciated that the use of shock waves can be used to treat calcification or similar lesions in other tissues and anatomy (e.g., structural heart walls and valves). IVL devices vary in design with respect to the energy source used to generate the acoustic shock waves, with two exemplary energy sources being electrohydraulic generation and laser generation.

For electrohydraulic generation of ultrasonic short pressure pulses, a conductive solution (e.g., saline) can be contained within an enclosure that surrounds electrodes or can be flushed through a tube that surrounds the electrodes. The calcified plaque modification is achieved by creating ultrasonic shock waves within the catheter by an electrical discharge (e.g., a plasma arc) across the electrodes. The energy from this electrical discharge enters the surrounding fluid, generating an acoustic shock wave where the wave itself is ultrasonic (i.e., a wave that has frequency components of greater than 20,000 Hz). In addition, the discharge creates one or more rapidly expanding and collapsing vapor bubbles that generate secondary shock waves due to the cavitation of the collapsing vapor bubble. The shock waves propagate radially outward and modify calcified plaque within the blood vessels. The shock waves travel deeply and safely through soft arterial tissue because of the acoustic impedance of soft tissue, which is similar to water. Acoustic impedance is a function of the density and the elasticity of a material and the speed of sound through that material. When the shock waves encounter tissues with a different acoustic impedance, such as intimal calcification of plaque close to the surface or endothelium of a vessel or medical calcification in the smooth muscle layer of a vessel, the leading edge of the shock wave imparts compressive stress on the calcified tissue. Shearing occurs on the lesion as the shock wave passes through the calcification. When the shock wave reaches the distal boundary of the calcification, the shock wave is both transmitted and reflected, inducing tensile stress that pulls the calcification apart. Further compressive stress is applied by squeezing, which occurs when the ultrasonic shock wave entering the calcium propagates faster than the remaining shock wave travelling outside the calcified region of tissue. These forces generated by IVL result in multi-plane and longitudinal fractures of the calcification in the tissue.

More specifically, catheters to deliver IVL therapy have been developed that include pairs of electrodes for electrohydraulically generating shock waves inside an angioplasty balloon. Shock wave devices can be particularly effective for treating calcified plaque lesions because the acoustic pressure from the shock waves can crack and disrupt lesions near the angioplasty balloon without harming the surrounding tissue. In these devices, the catheter is advanced over a guidewire through a patient's vasculature until it is positioned proximal to and/or aligned with a calcified plaque lesion in a body lumen. The balloon is then inflated with conductive fluid (e.g., using a relatively low pressure of 2-4 atm) so that the balloon expands to contact the lesion but not to a degree that substantively displaces the lesion. Voltage pulses can then be applied across the electrodes of electrode pairs to produce acoustic shock waves that propagate through the walls of the angioplasty balloon and into the lesions. Once the lesions have been cracked by the acoustic shock waves, the balloon can be expanded further to increase the cross-sectional area of the lumen and improve blood flow through the lumen. Alternative devices to deliver IVL therapy can include electrodes disposed within a closed volume other than an angioplasty balloon, such as a cap, balloons of variable compliancy, or other type of enclosure.

The calcified plaque remains in place following application of the shock waves; intimal calcium remains in the blood vessel lining and medical calcium remains in the muscle tissue surrounding the blood vessel. IVL generally does not cause the debulking or extirpation of tissue from a blood vessel wall. However, following the IVL shock wave, the hardened lesion is fractured and does not have the mechanical strength to resist against the expansion of a balloon. Thus, following delivery of IVL shock wave therapy, in some approaches, the catheter can be expanded or moved such that the modified underlying lesion can be moved or displaced along the blood vessel, similar to how a plain angioplasty balloon can treat non-calcified plaque. In some approaches, expansion of the balloon can be done in sequence with or concurrent with delivery of the IVL therapy.

Accordingly, the IVL process can also be considered different from standard atherectomy procedures and different from cutting or scoring balloons at least in that IVL cracks calcium but does not liberate the calcium from the tissue. Hence, generally speaking, IVL systems should not require aspiration nor embolic protection. Accordingly, IVL does not carry the same degree of risk of embolism, perforation, dissection, or other damage to vasculature as atherectomy procedures or angioplasty procedures using cutting or scoring balloons. In further contrast with cutting techniques, due to the compliance of a normal blood vessel and non-calcified plaque, the shock waves produced by IVL do not modify the normal healthy vessel tissue or non-calcified plaque. In other words, the shock waves from IVL do not have an adverse clinical impact on soft tissues while treating the hardened calcified anatomy.

For laser generation of acoustic shock waves, a laser pulse is transmitted into and energy from the laser is absorbed by a fluid within the catheter, optionally with a target to act as catalyst for the laser absorption. This absorption process rapidly heats and vaporizes the fluid, thereby generating the rapidly expanding and collapsing vapor bubble, as well as the acoustic shock waves that propagate outward and modify the calcified plaque. The acoustic shock wave intensity is higher if a fluid is chosen that exhibits strong absorption at the laser wavelength that is employed. These examples of electrohydraulic and laser-based IVL devices are not intended to be a comprehensive list of potential energy sources to create the ultrasonic IVL shock waves.

Described herein are devices, systems, and methods for concentrating forward-biased shock waves using a nozzle disposed at a distal end of a catheter to provide targeted, high-powered sonic output to a treatment area located distally of the catheter. In some examples, the catheters described herein include a catheter body and at least one shock wave emitter disposed at a distal end of the catheter body. A nozzle is disposed at the distal end and configured such that shock waves generated by the at least one shock wave emitter propagate within the nozzle and are directed to the nozzle outlet.

The nozzles described herein are configured such that the shock waves (and/or bubbles resulting from shock wave generation) concentrate at the outlet of the nozzle. As the shockwaves propagate within the nozzle moving toward the outlet, fluid within the nozzle is pushed forward toward the nozzle outlet, increasing in pressure and velocity as it approaches the outlet of the nozzle. The concentrated shock waves (and/or bubbles) along with the accelerated fluid may be directed distally of the catheter body via the nozzle outlet toward a target treatment area to break up, fragment, or otherwise impact on calcified tissue, fibrotic tissue, tissue having more than one morphology (“multi-morphology”, e.g., a combination of calcified and fibrotic tissue), or other lesions within a body lumen.

Catheters configured with a nozzle according to the principles described herein may be used in pacemaker lead removal. The nozzle may be configured to receive a pacemaker lead wire. The catheter may be guided by the pacemaker lead wire to a location of fibrotic or multi-morphology tissue formed on and/or around the pacemaker lead wire. Shock waves generated by the catheter are concentrated by the nozzle onto tissue formed on the pacemaker lead wire and located within the nozzle (such as at the outlet) and/or in front of the nozzle outlet to assist in releasing the pacemaker lead wire from cardiac tissue. The nozzle can be configured such that a uniform pressure is applied to the pacemaker lead to minimize the possibility of fragmentation and/or breakage of the lead. The nozzle may include a beveled or tapered edge, which can be used to scrape or otherwise remove fibrotic and calcified tissue from the lead as the catheter is advanced within the body lumen.

Nozzles described herein may provide a variety of technical advantages. For instance, concentrating shock waves using the nozzles described herein can focus sonic energy on a smaller target treatment area, increasing the treatment energy applied to the target treatment area. When applied to pacemaker leads, this focused energy can help disintegrate calcifications, fibrotic scar tissue, and/or adhesions that may have formed around the leads. The nozzles also enable precise control over the sonic energy flow, allowing medical professionals to target specific areas around the pacemaker leads more accurately and ensuring that the energy is applied only where needed. This level of control is beneficial in preventing damage to surrounding tissues or structures. Moreover, the nozzles may ensure that the pressure exerted on the pacemaker leads is uniform, reducing the risk of uneven treatment or damage to the leads themselves. This uniformity may enable a safe and effective removal process. For instance, the nozzles may reduce the probability of lead fragmentation, which can become problematic when fragments migrate to other parts of the body.

By optimizing the efficacy of lithotripsy, catheters incorporating nozzles, according to the principles described herein, may decrease the likelihood of complications during treatment of lesions in the vasculature and urinary tract and during pacemaker lead removal procedures.

The enhanced precision and effectiveness of the lithotripsy procedure using the nozzles described herein can potentially lead to shorter overall procedure times. This benefits both patients, who experience less time in a medical setting, and healthcare providers, who can serve patients more efficiently.

Also disclosed are devices and methods that provide directional control of shock-wave propagation. A distal cap may be positioned distally of one or more shock wave emitters, and a deflector may be positioned between a distal end of the cap and the shock wave emitters. The deflector may be positioned to redirect distally propagating energy, including shock waves, cavitation bubbles, and/or fluid flow, radially toward a target site. The distal cap may include an aperture that allows fluid, cavitation bubbles, and/or shock waves to propagate outwardly from the distal cap in a radial direction. During operation, the deflector redirects the shock waves, cavitation bubbles and/or fluid flow at least partially radially outward through the aperture toward a lesion or other treatment site. This directional control may enable enhanced range of treatment via simple rotation of the catheter within the body lumen. Moreover, allowing fluid and/or cavitation bubbles to exit via an aperture and impinge on a treatment site may enhance the therapeutic effect of shock wave treatment.

It is to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes,” “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof. As provided herein, it should be appreciated that any disclosure of a numerical range describing dimensions or measurements such as thicknesses, length, weight, time, frequency, temperature, voltage, current, angle, etc. is inclusive of any numerical increment or gradient within the ranges set forth relative to the given dimension or measurement. It should be further appreciated that any disclosure of a numerical range as a boundary term or inequality term is similarly inclusive of any numerical increment or gradation within the given range; e.g., recitation of a parameter that is “at least a defined value, where the defined value ranges from 5% to 50%” supports the disclosure of that parameter being “at least 5%”, “at least 50%”, “at least 37%”, “at least 42.4%”, and the like. Furthermore, numerical designators such as “first,” “second,” “third,” “fourth,” etc. are merely descriptive and do not indicate a relative order, location, or identity of elements or features described by the designators. For instance, a “first” shock wave may be immediately succeeded by a “third” shock wave, which is then succeeded by a “second” shock wave. As another example, a “third” emitter may be used to generate a “first” shock wave and vice versa. Accordingly, numerical designators of various elements and features are not intended to limit the disclosure and may be modified and interchanged.

As used herein, the term “electrode” refers to an electrically conducting element (typically made of metal) that receives electrical current and subsequently releases the electrical current to another electrically conducting element. In the context of the present disclosure, electrodes can be positioned relative to each other, such as in an arrangement of an inner electrode and an outer electrode. Accordingly, as used herein, the term “electrode pair” refers to two electrodes that are positioned in close proximity such that application of a sufficiently high voltage to the electrode pair can cause an electrical current to transmit across a gap (also referred to as a “spark gap”) between the two electrodes (e.g., from an inner electrode to an outer electrode, or vice versa), such as by passing through a conductive fluid or gas. In the context of the present disclosure, the term “emitter” can refer to one or more electrode pairs formed at a particular location (such as longitudinally) along a length of axis or catheter. In some contexts, one or more electrode pairs may also be referred to as an electrode assembly, which may include one or more emitters, and which broadly refers to the region where current transmits across one or more electrode pairs, generating at least one shock wave.

Components of emitters, including electrodes and various planar emitter structures, may be formed from a metal, such as stainless steel, copper, tungsten, platinum, palladium, molybdenum, cobalt, chromium, iridium, an alloy or alloys thereof, such as cobalt-chromium, platinum-chromium, cobalt-chromium-platinum-palladium-iridium, or platinum-iridium, or a mixture of such materials.

The voltage pulse applied by a power source, including any of the power sources described herein (which may also be referred to herein as voltage sources or pulse generators), can be in the range of from about five hundred to three thousand volts (500 V-3,000 V). In some implementations, for the treatment of stenosis in a blood vessel or of another anatomical feature, the voltage pulse applied by the voltage source can be up to about fifteen thousand volts (15,000 V) or higher than fifteen thousand volts (15,000 V). The pulse width of the applied voltage pulses ranges between one microsecond and six microseconds (1-6 μs). The repetition rate or frequency of the applied voltage pulses may be between about 1 Hz and 10 Hz. The total number of pulses applied by the power source to a treatment device (e.g., an IVL catheter) can be, for example, sixty (60) pulses, eighty (80) pulses, one hundred twenty (120) pulses, three hundred (300) pulses, or up to five hundred (500) pulses, or any increments of pulses within this range. Further implementations of power sources can deliver greater than 500 pulses to a treatment device. Alternatively or additionally, in some examples the power source may be configured to deliver a packet of micro-pulses having a sub-frequency between about 10 Hz-10 kHz. The preferred voltage, repetition rate, and number of pulses for any given IVL device or treatment may vary depending on factors such as the size, length, eccentricity, nodularity, or orientation of the lesion, the extent of lesion or tissue calcification, the size of the blood vessel, the attributes of the patient (e.g., age, gender, predisposition to cardiac disease, etc.), or the stage of treatment. In delivering a treatment regime, a physician may start with low energy shock waves and increase the energy as needed during the procedure, or vice versa. The amount of power delivered for shock waves may further vary during the course of a procedure, following a predetermined sequence of energy increases or decreases, or by changing the amount of energy delivered in response to sensor data obtained prior to and/or during the IVL treatment procedure. The magnitude of the shock waves can be controlled by controlling the voltage, current, duration, and repetition rate of the pulsed voltage from the power source.

In some implementations, an IVL catheter may be a “rapid exchange-type” (“RX”) catheter provided with an opening portion located along the length of the catheter through which a guidewire can be directed (such as through a middle portion of a central tube in a longitudinal direction). In some other implementations, an IVL catheter may be an “over-the-wire-type” (“OTW”) catheter in which a guidewire lumen is formed throughout the overall length of the catheter, and a guidewire can be guided through the proximal end of a hub. A guidewire lumen entry point to a catheter is at or proximate to the distal end of the catheter tip, and the guidewire lumen extends through a portion of the catheter to an exit port. Thus in use, a guidewire is delivered into the anatomy of a patient, the proximal end of the guidewire (outside the patient) is fed into the distal end opening of the catheter, and the catheter is run along the guidewire until it reaches the target tissue at the distal end of the guidewire (inside the patient); the effective difference between an OTW and an Rx catheter is where the guidewire exits the catheter. The selection between an OTW design and an Rx design is driven by factors including (but not limited to): anatomy to be treated (e.g., coronary vasculature vs. peripheral vasculature); the length of guidewire to be used; the trackability, stiffness, torque transmission, and deliverability of the catheter; the profile and cross-section of the catheter, the ability to exchange a wire when the catheter is past a stenosis; positioning of the distal end of a catheter close to the end of a guidewire and further obtaining positional confirmation of the catheter.

Certain standard anatomical terms of location may be used herein to refer to the anatomy of animals, and namely humans, with respect to the example implementations. Although certain spatially relative terms, such as “outer,” “inner,” “upper,” “lower,” “below,” “above,” “vertical,” “horizontal,” “top,” “bottom,” and similar terms, are used herein to describe a spatial relationship of one element, device, or anatomical structure to another device, element, or anatomical structure, it is understood that these terms are used herein for ease of description to describe the positional relationship between elements and structures, as illustrated in the drawings. It should be understood that spatially relative terms are intended to encompass different orientations of the elements or structures, in use or operation, in addition to the orientations depicted in the drawings. For example, an element or structure described as “above” another element or structure may represent a position that is below or beside such other element or structure with respect to alternate orientations of the subject patient, element, or structure, and vice-versa. As used herein, the term “patient” may generally refer to humans, anatomical models, simulators, cadavers, and other living or non-living objects.

In the following description of the various embodiments, reference is made to the accompanying drawings, in which are shown, by way of illustration, specific embodiments that can be practiced. It is to be understood that other embodiments and examples can be practiced, and changes can be made without departing from the scope of the disclosure.

Efforts have been made to improve the design of electrode assemblies included in shock wave and directed cavitation catheters. For instance, low-profile electrode assemblies have been developed that reduce the crossing profile of a catheter and allow the catheter to more easily navigate calcified vessels to deliver shock waves in more severely occluded regions of vasculature. Examples of low-profile electrode designs can be found in U.S. Pat. Nos. 8,888,788, 9,433,428, and 10,709,462, in U.S. Publication No. 2021/0085383, and in U.S. patent application Ser. No. 18/586,299, all of which are incorporated herein by reference in their entireties. Other catheter designs have improved the delivery of shock waves, for instance, by specific electrode construction and configuration thereby directing shock waves in a forward direction to break up tighter and harder-to-cross occlusions in vasculature. Examples of forward-biased or firing-firing catheter designs can be found in U.S. Pat. Nos. 10,966,737, 11,478,261, and 11,596,423, in U.S. Publication Nos. 2023/0107690 and 2023/0165598, and in U.S. Patent Application Ser. No. 18/524,575 and Ser. No. 18/680,853, all of which are incorporated herein by reference in their entireties.

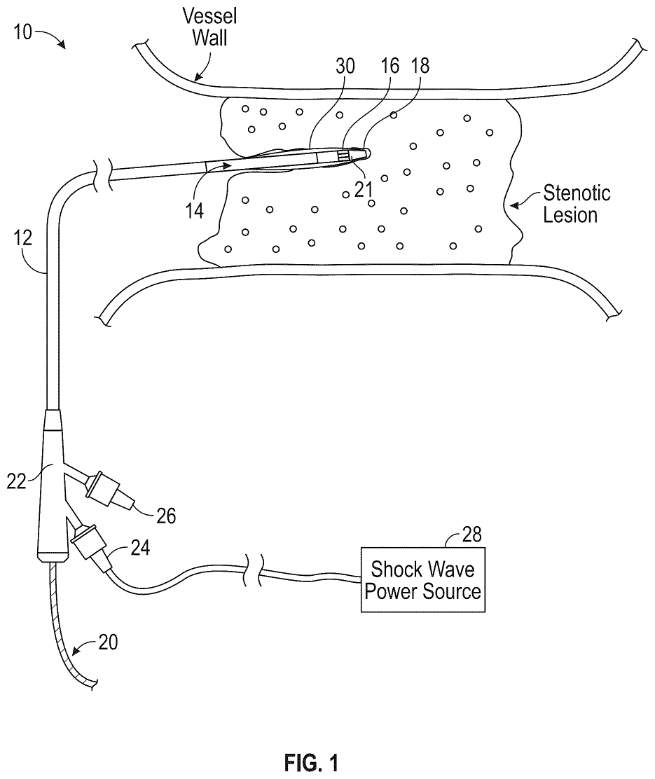

FIG. 1 illustrates a system for treating calcifications in body lumens. The system includes a shock wave generating catheter 10. The catheter 10 may be used to fragment, crack, or otherwise break up calculi within a body lumen, for instance, to treat various occlusions within blood vessels. The catheter 10 includes at its distal end at least one shock wave emitter 16 and a nozzle 18. The catheter 10 is advanced into an occlusion in a patient's vasculature, such as the stenotic lesion depicted in FIG. 1, over a guidewire 20, which may be carried in a guidewire sheath and voltage pulses are applied to the at least one shock wave emitter 16 to generate shock waves that propagate out through the nozzle 18 distally of the distal end of catheter 10. In some examples, a side saddle, guide catheter or guide sheath, or micro guide wire may be used in place of the guide wire 20.

The at least one shock wave emitter may include an electrode pair having first and second electrodes separated by a gap, at which shock waves are formed when a current flows across the gap between the electrodes of the pair (i.e., when a voltage is applied across the first and second electrodes). The electrode pairs described herein may be formed by an emitter band and one or more electrodes positioned adjacent to the emitter band, between adjacent exposed portions of two conductive wires, or otherwise by two conductive elements positioned adjacent to one another separated by a spark gap.

The nozzle 18 disposed at the distal end of the catheter 10 is configured such that at least one shock wave and/or bubble generated by the at least one shock wave emitter propagates nozzle 18 and is concentrated at an outlet of the nozzle and directed distally of the catheter 10. For instance, the nozzle may be formed from an acoustically reflective material such that shock waves and/or bubbles directed into the nozzle are reflected by the nozzle wall 21. The shock waves may continue to propagate forward (e.g., distally) within the nozzle as they reflect from converging walls of the nozzle, reaching a peak concentration at an outlet of the nozzle before propagating distally of the catheter through the nozzle outlet. This shock waves propagating within the nozzle may cause a fluid within the nozzle to move toward the nozzle outlet, increasing in pressure and velocity as it propagates within the nozzle toward the outlet. The fluid may exit via the nozzle outlet and project forward toward a target treatment area.

Accordingly, in some examples, the nozzle 18 includes an open outlet at its distal end through which fluid can exit. A fluid port 26 may be connected to a fluid supply line and a fluid return line extending along at least a portion of the catheter body to an inlet of the nozzle. During shock wave generation, fluid may exit the nozzle from the nozzle outlet at the distal end. The fluid supply line connected to fluid port 26 may be configured to supply a fluid (e.g., saline or other conductive fluid) to an inlet of the nozzle to replace fluid that exits the outlet of the nozzle when the respective shock waves are generated by the one or more shock wave emitters. Debris from shock wave generation (e.g., fragmented calcified tissue) may collect in the nozzle and vasculature following shock wave generation. The fluid return line connected to fluid port 26 may be configured to remove debris from the body lumen received via the outlet of the nozzle.

In some examples, an enclosure 30 (e.g., a low-profile flexible angioplasty balloon, a polymer membrane in tension that can flex outward, etc.) may optionally be sealably attached to the distal end 14 of the catheter 10, forming a channel around the shaft 12 of the catheter. The enclosure 30 may surround the plurality of shock wave emitters 16 and nozzle 18, such that the shock waves are produced in a closed system within the enclosure 30. The enclosure 30 may be filled or inflated with a conductive fluid, such as saline. The enclosure 30 can alternatively be referred to as a “window”, in particular for implementations where when the interior volume is filled with a fluid and pressurized, the window maintains a substantively constant volume and profile. The conductive fluid allows the acoustic shock waves to propagate distally from the electrode pair(s) of the shock wave emitter(s) 16 through the walls of the enclosure 30 and then into the target lesion. In one or more examples, the conductive fluid may also contain x-ray contrast fluid to permit fluoroscopic viewing of the catheter 10 during use. In some implementations, the material that forms the primary surface(s) of the enclosure 30 through which shock waves pass can be a noncompliant polymer. In other implementations, a rigid and inflexible structure may be used in lieu of enclosure 30. The enclosure 30 may mitigate thermal injury to soft tissue and reduce cavitation stresses by limiting expansion of the vapor bubbles produced during shock wave generation to the interior of the enclosure. For instance, the vapor bubbles hit the enclosure wall before reaching their maximum potential size, thus inducing collapse, and reducing cavitation stress and preventing soft tissue injury that can be caused by tensile stresses during cavitation bubble collapse.

The catheter 10 includes a proximal end 22 (or handle) that remains outside of a patient's vasculature during treatment. The proximal end 22 includes an entry port for receiving the guidewire 20. The proximal end 22 also includes the fluid port 26 for receiving a conductive fluid for filling and emptying the nozzle 18 and/or the enclosure 30 during treatment. An electrical connection port 24 is also located on the proximal end 22 to provide an electrical connection between the distal shock wave emitters 16 and an external pulsed high voltage source 28, such as the intravascular lithotripsy (IVL) generator shown in FIG. 1. In some embodiments, generator 28 may be configured to deliver the voltage pulses at a rate of between 1 Hz and 100 Hz, including 1 Hz and 100 Hz. In some embodiments, generator 28 is configured to deliver the voltage pulses at a rate of a rate of between 1 Hz and 50 Hz, including 1 Hz and 50 Hz. Shock wave energy generator 530 may be configured to deliver the voltage pulses at a rate of a rate of up to 100 Hz, up to 90 Hz, up to 80 Hz, up to 70 Hz, up to 60 Hz, up to 50 Hz, up to 40 Hz, up to 30 Hz, up to 20 Hz, and/or up to 10 Hz. Shock wave energy generator 530 may be configured to deliver the voltage pulses at a rate of at least 10 Hz, at least 20 Hz, at least 30 Hz, at least 40 Hz, at least 50 Hz, at least 60 Hz, at least 70 Hz, at least 80 Hz, at least 90 Hz, and/or at least 100 Hz.

Generator 28 may be a portable and/or rechargeable voltage source. Generator 28 may be a laser pulse generator. Generator 28 may be configured to deliver high voltage pulses between 3 kV and 30 kV, including 3 kV and 30 kV. In some embodiments, the high voltage pulses are between 10 kV and 20 kV, including 10 kV and 20 kV. In some embodiments, the high voltage pulses are between 15 kV and 20 kV, including 15 kV and 20 kV. In some embodiments, the high voltage pulses are greater than 20 kV. The high voltage pulses may be at least 1 kV, at least 2 kV, at least 3k V, at least 4k V, at least 5 kV, at least 6k V, at least 7 kV, at least 8 kV, at least 9 kV, at least 10 kV, at least 11 kV, at least 12 kV, at least 13 kV, at least 14 kV, at least 15 kV, at least 16 kV, at least 17 kV, at least 18 kV, at least 19 kV, at least 20 kV, and/or at least 30 kV. The high voltage pulses may be no more than 30 kV, no more than 20 kV, no more than 19 kV, no more than 18 kV, no more than 17 kV, no more than 16 kV, no more than 15 kV, no more than 14 kV, no more than 13 kV, no more than 12 kV, no more than 11 kV, no more than 10 kV, no more than 9 kV, no more than 8 kV, no more than 7 kV, no more than 6 kV, no more than 5 kV, no more than 4 kV, no more than 3 kV, no more than 2 kV, and/or no more than 1 kV.

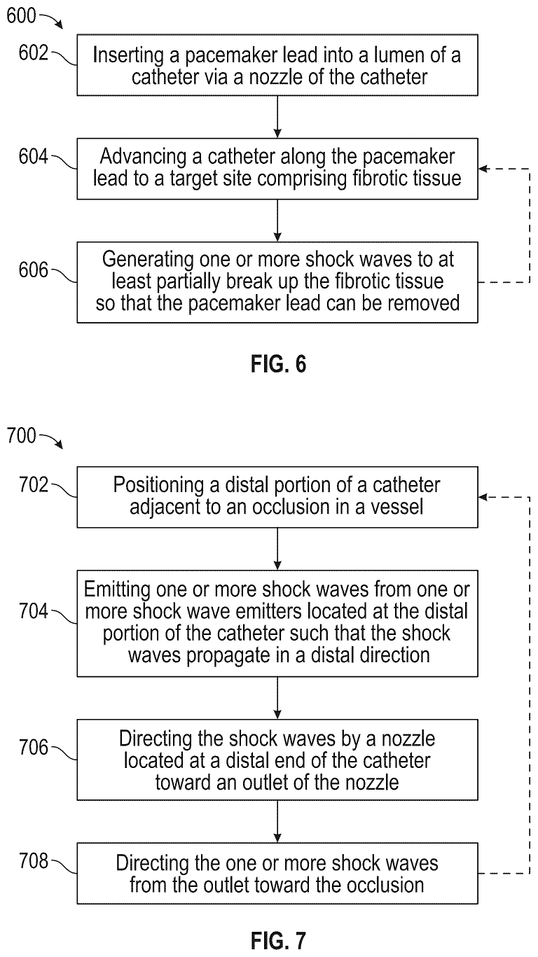

The catheter 10 also includes a flexible shaft 12 that extends from the proximal end 22 to the distal end 14 of the catheter. The shaft 12 provides various internal conduits connecting elements of the distal end 14 with the proximal end 22 of the catheter (see, e.g., FIG. 6D for a cross-section of a region an exemplary shaft). The shaft 12 includes an elongate tube that includes a lumen for receiving the guidewire 20. The elongate tube may include additional lumens extending through the shaft 12 or along an outer surface of the shaft 12. For example, one for fluid lumens (e.g., a fluid inlet lumen and a fluid outlet lumen or a combined flush lumen) can be located along or within the shaft 12 for carrying conductive fluid from the fluid port 26 into the enclosure 30.

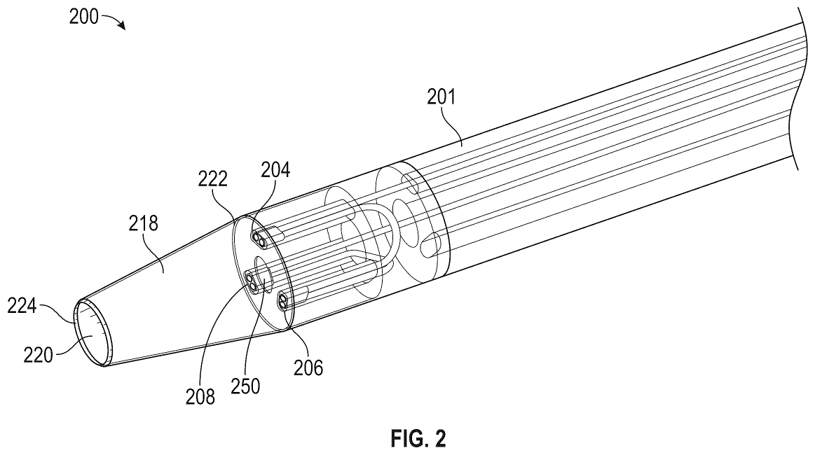

FIG. 2 illustrates an isometric view of an exemplary catheter 200 that can be used for catheter 10 of FIG. 1. Catheter 200 includes a catheter body 201 and a nozzle 218 positioned distally of a plurality of shock wave emitters 204, 206, and 208 at a distal end of catheter 200. Although catheter 200 is illustrated as having three shock wave emitters positioned at the distal end, it should be understood that any number of shock wave emitters may be positioned at or near the distal end of catheter 200 and/or along the length of catheter body 201. The nozzle 218 may be configured such that shock waves generated by shock wave emitters 204, 206, and 208 propagate toward an outlet 220 of the nozzle 218. The nozzle 218 may be configured to concentrate the at least one shock wave generated by shock wave emitters 204, 206, and 208 at the outlet of the nozzle. For instance, the nozzle 218 may be a convergent nozzle (e.g., may include an outlet 220 that has a smaller diameter than its inlet 222).

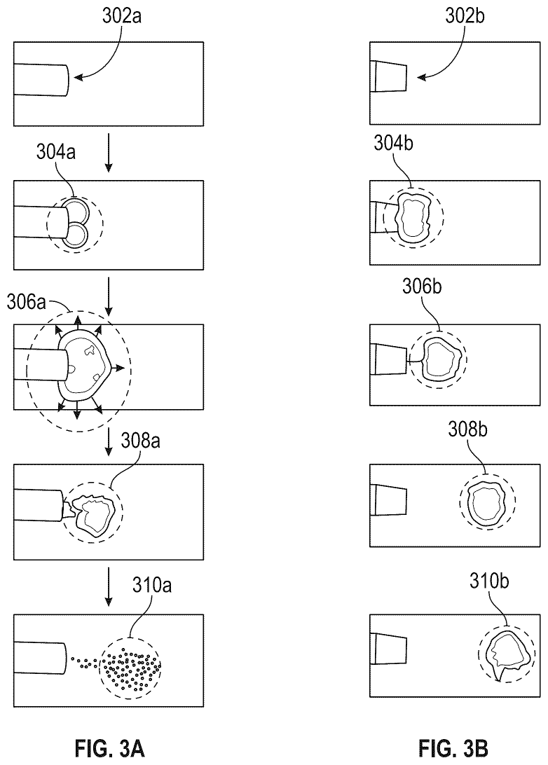

In some examples, one or more bubbles may be generated as a result of the shock wave generation process. Nozzle 218 may be configured to direct at least one bubble to the outlet of the nozzle. The nozzle 218 may be configured to concentrate the at least one bubble at the outlet 220 of the nozzle such that the at least one bubble propagates distally of the outlet 220. A maximum concentration of bubbles and/or shock waves may occur at the outlet 220 of the nozzle 218. Although other forward-directed IVL shock wave generating devices (e.g., devices with emitters that produce primarily distally propagating shock waves) without a nozzle also produce bubbles during the shock wave generation process, those bubbles may collapse shortly after formation. The nozzle 218 may enable the concentrated bubble(s) to propagate further toward a target treatment area, for instance, as shown in FIG. 3B, described below. The collapse of the bubble(s) at or near the treatment area may enhance the effect of forces produced via the collapse on breaking up calcifications, fibrotic tissue, multi-morphology tissue, or other lesions.

In some examples, a diameter of the nozzle outlet 220 may be at least as large as a central guide wire lumen 250 extending along the length of catheter body 220 such that a guide wire can extend through the nozzle outlet. In at least some examples, a diameter of nozzle outlet 220 may be configured to receive a pacemaker lead. In some examples, the nozzle outlet 220 has a diameter of 3 millimeters (mm) to 15 millimeters (mm). In some examples, the nozzle outlet 220 has a diameter of at least 5 millimeters (mm). In some examples, the nozzle outlet 220 has a diameter of less than 10 millimeters (mm). In some examples, nozzle 218 outlet 220 may be configured such that a pacemaker lead can be inserted through outlet 220 and into the central guide wire lumen 250. In some examples the outlet 220 of nozzle 218 includes a beveled edge (e.g., for removing tissue from a pacemaker lead wire). However, the outlet 220 may be formed into a variety of other geometries. For instance, the outer edge of outlet 220 may be rounded or flattened to provide a protective surface for tissues as the catheter is navigated through a body lumen. Nozzle 218 may be configured such that its widest diameter is sufficiently narrow to navigate through vasculature. In some examples, the widest diameter of nozzle 218 may be flush with an outer diameter of the catheter body 201. In some examples the widest diameter of nozzle 218 may be wider than an outer diameter of the catheter body 201 to accommodate shock wave emitters that are positioned radially outward of an outer diameter of catheter body 201. In some examples, the widest diameter of nozzle 218 is less than 15 millimeters (mm). In some examples, the widest diameter of nozzle 218 is less than 9 millimeters (mm). In some examples, the widest diameter of nozzle 218 is 7 millimeters (mm) to 10 millimeters (mm). In at least some examples, the nozzle outlet 220 has a diameter of between 0.355 millimeters (mm) and 0.457 millimeters (mm). The diameter of the nozzle outlet may be at least 0.2 millimeters (mm), at least 0.3 millimeters (mm), at least 0.4 millimeters (mm), at least 0.5 millimeters (mm), at least 1 millimeter (mm), or at least 2 millimeters (mm). The nozzle outlet may be at most 2 millimeters (mm), at most 1 millimeter (mm), at most 0.5 millimeters (mm), at most 0.4 millimeters (mm), at most 0.3 millimeters (mm), or at most 0.2 millimeters (mm).

In some examples, nozzle 218 may extend distally of any shock wave emitters and have a tapered distal end. In some examples, nozzle 218, at nozzle outlet 220, has a diameter that is less than at a proximal end of nozzle 218. This structure may allow for the distal, therapy-delivering end of the device to be narrower than proximal regions of the device.

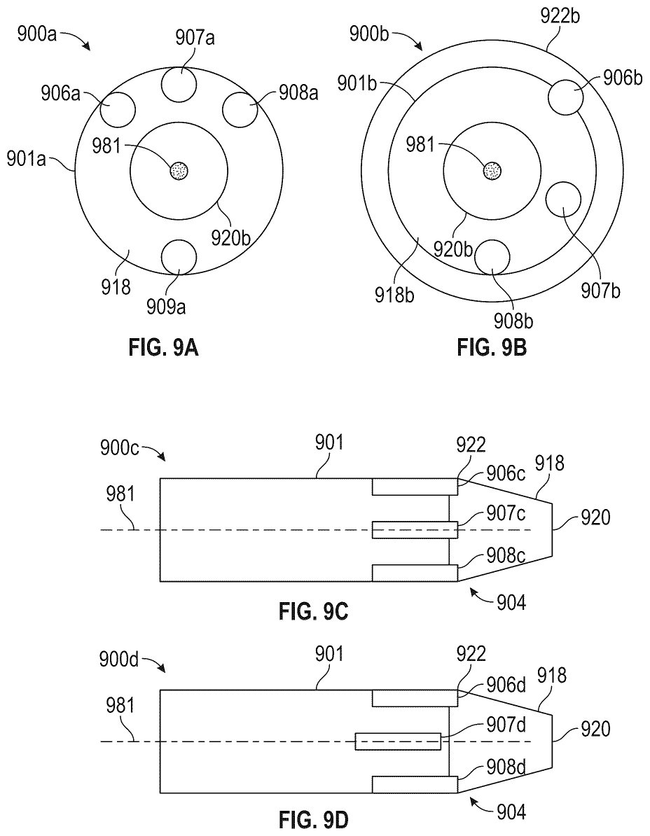

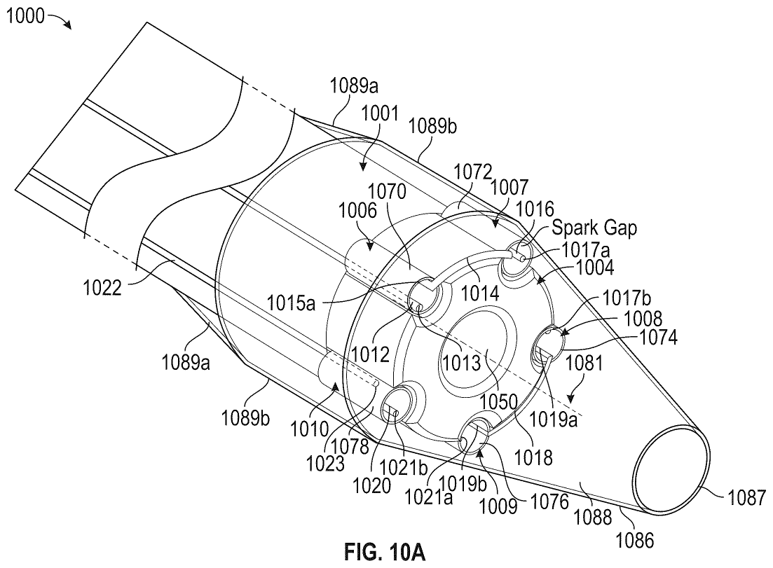

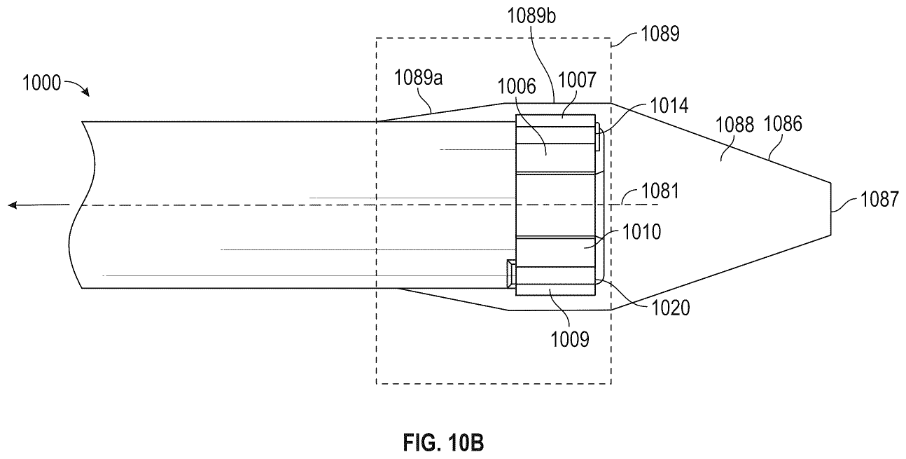

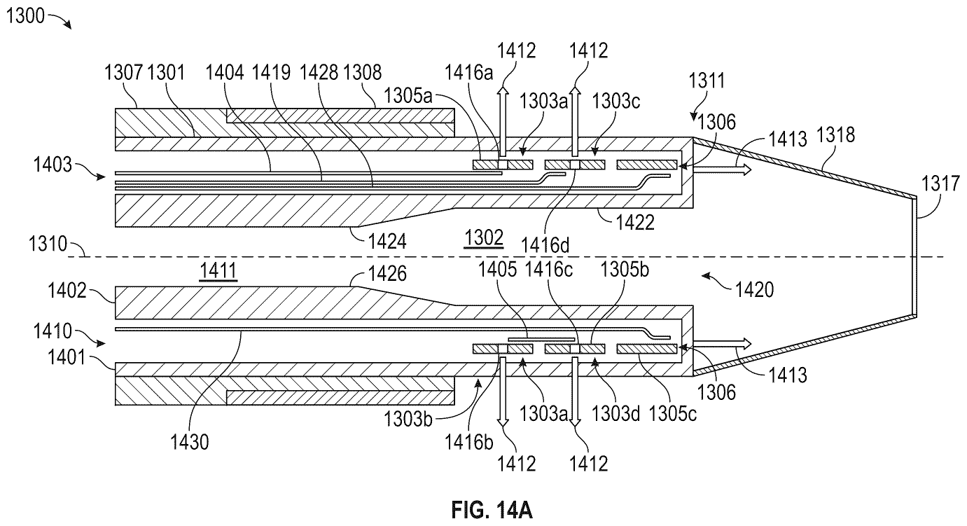

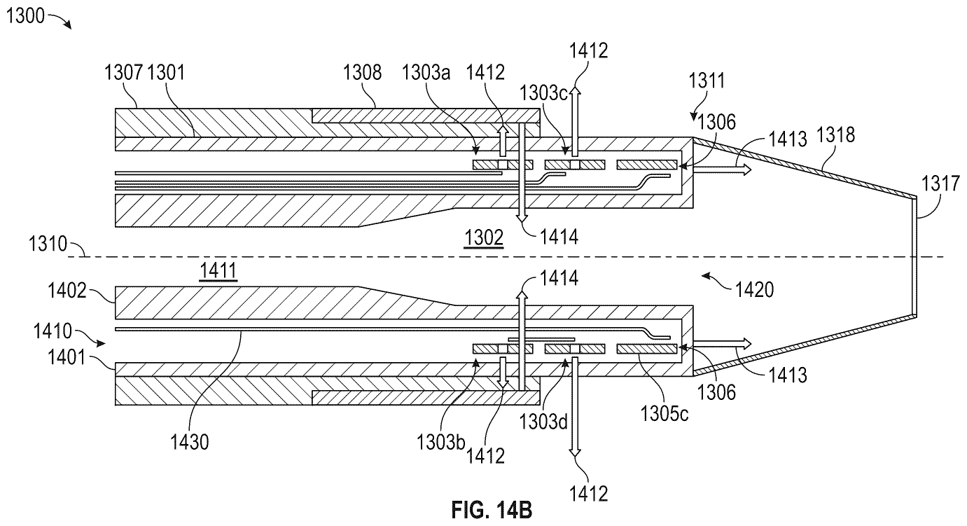

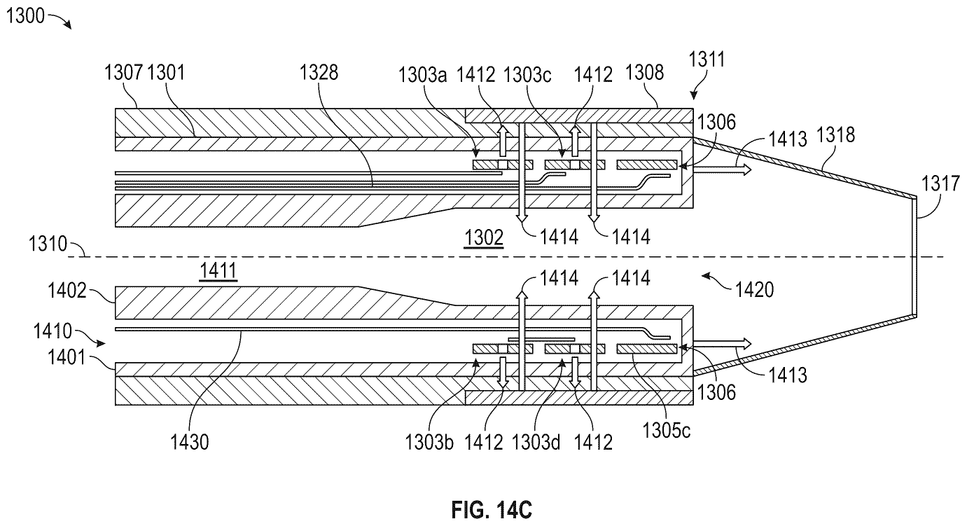

In some examples, the nozzle may be positioned such that shock waves emitted from shock wave emitters 204, 206, and 208 are formed within an interior space of nozzle 218. The nozzle may be configured such that the shock waves are formed near a proximal end/inlet 222 of the nozzle and propagate toward an outlet 220 at its distal end. For instance, the nozzle 218 may be positioned such that its inlet 222 is longitudinally aligned on the catheter body with the shock wave emitters 204, 206, and 208. Nozzle 218 of catheter 200 may positioned such that shock wave emitters 204, 206, and 208 are positioned radially inward of an outer diameter of an inlet to the nozzle. Positioning the emitters 204, 206, and 208 radially inward of the nozzle ensures that any radially biased portion of the shock waves to reflect inwardly of nozzle 218 as they propagate away from the distal end of catheter body 201. In some examples, as described throughout, one or more shock wave emitters may instead be positioned distally of a nozzle inlet and/or otherwise partially within the nozzle (e.g., as illustrated in FIGS. 9C, 9D, 10A and 10B). In some examples, one or more radially-biased shock wave emitters may be positioned proximally of the nozzle and may be reflected into the nozzle using acoustically reflective surfaces (e.g., as illustrated in FIGS. 14A-C). Various modifications to the relative positioning of the nozzle and emitters, and to the shape of the nozzle and/or emitters are described throughout and may impact the manner in which the shock waves propagate within the nozzle and distally of the nozzle outlet.

Nozzle 218 may be formed at least in part of an acoustically reflective and biocompatible material. For instance, nozzle 218 may include medical-grade plastics, steel, or other non-reactive materials. Nozzle 218 may be formed from stainless-steel, high-density polyethylene, polyvinyl chloride, polyether ether ketone (PEEK), or a combination thereof. Stainless steel may be preferred in some examples due to its ability to capture and thrust forward all generated by the emitters, while also exhibiting less attenuation of energy compared to plastic alternatives. In some examples, the distal end of catheter body 201 is also formed of an acoustically reflective material such that any proximally propagating portion of shock waves generated by emitters 204, 206, and 208 are reflected into nozzle 218.

In some examples, the nozzle may be attached to a distal end of a catheter by cutting slits into a proximal end of the nozzle, compressing the portions separated by the slits over the distal end of the catheter, and using a laser welder to weld the slits together on the distal end of the nozzle. Nozzles may be formed via injection molding, formed with an RF tipping machine, and/or reflowed over a shaped mandrel using Heatshrink and hot air. Metal nozzles may be swaged or stamped.

FIGS. 3A and 3B illustrate a side-by-side comparison of bubble propagation without a nozzle (FIG. 3A) and with a nozzle (FIG. 3B). In FIG. 3A, block 302a illustrates a catheter with a plurality of shock wave emitters positioned at a distal end of the catheter prior to shock wave generation. Block 304a illustrates the catheter immediately after application of a voltage pulse to the shock wave emitters of the catheter. As shown in block 304a, at least two bubbles formed as a result of the shock wave generation process. In block 306a, the at least two bubbles have merged into a larger bubble that is expanding outwardly while still positioned immediately adjacent to the catheter's distal end. The outward expansion leads to the rapid collapse of the bubble illustrated in block 308a. Very little forward propagation of the bubble occurs prior to collapse in 308a without a nozzle to concentrate the bubble. Block 310a illustrates many small bubbles formed following collapse of the combined bubble in 306a propagating distally of the catheter.

FIG. 3B illustrates catheter with a plurality of shock wave emitters and a nozzle positioned at a distal end of the catheter prior to shock wave generation. Block 302b illustrates the catheter with a plurality of shock wave emitters and nozzle positioned at a distal end of the catheter prior to shock wave generation. Block 304b illustrates concentrated bubble(s) propagating distally outward from an outlet of the nozzle following shock wave generation. Similar to the catheter of FIG. 3A, cavitation bubbles formed at each of the shock wave emitters may combine to form a single larger cavitation bubble. As shown in blocks 306b-310b, the concentrated bubble(s) formed at the outlet of the nozzle propagate distally farther than those formed without the nozzle without collapsing. Depending on the dimensions of the nozzle, the cavitation bubble may also become elongated in the direction of propagation. The nozzle thus enables the concentrated bubble(s) to propagate to a target treatment area positioned further from the distal end of the catheter (relative to a device without a nozzle) prior to collapse. When a bubble collapses upon reaching a target treatment area, the forces resulting from its collapse are exerted on the tissue to break up calcifications, fibrotic tissue, multi-morphology tissue, or a combination thereof. Without a nozzle, the large bubble illustrated in FIG. 3A cannot reach the target treatment area unless the catheter is positioned immediately adjacent to the tissue to be treated. With the nozzle depicted in FIG. 3B, the bubble can reach the target treatment area even if the catheter cannot be sufficiently advanced within the body lumen to position it immediately adjacent to the tissue.

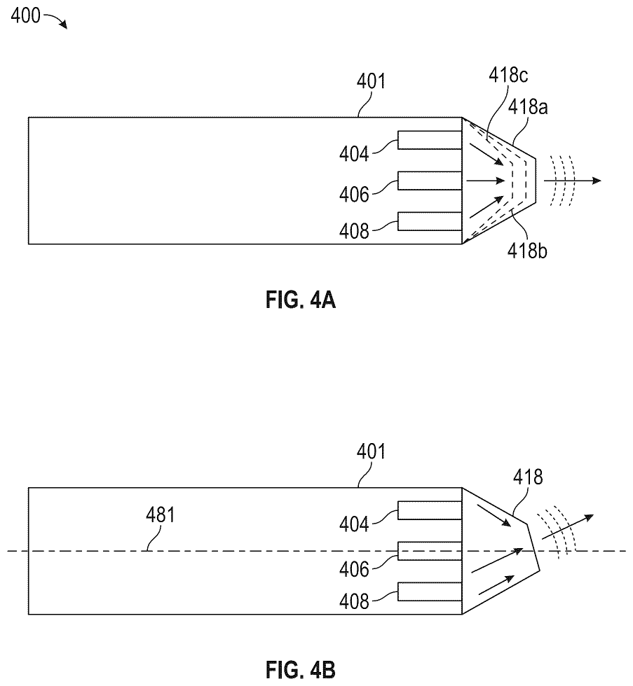

The nozzles described herein may include a variety of different shapes, lengths, inlet and outlet diameters, and so on. Nozzle design parameters may be adjusted based on shock wave emitter configurations and/or to provide a desired concentration of shock waves/bubbles at the nozzle outlet and distally of the nozzle outlet. For instance, some nozzles may include narrower outlets to provide greater shock wave concentration relative to wider outlets, some nozzles may be relatively longer than other nozzles and/or include a relatively steeper converging portion toward the outlet, and so on. FIGS. 4A and 4B illustrate exemplary nozzles having different design features. It should be understood that the examples depicted in FIGS. 4A and 4B are exemplary and in no way limiting. Numerous additional or different variations are within the scope of this disclosure.

FIG. 4A illustrates an exemplary catheter 400 that includes a catheter body 401, a plurality of shock wave emitters 404, 406, and 408, and a nozzle. Three different variations of the nozzle are illustrated and labeled 418a, 418b, and 418c, respectively. As shown, the length of the nozzle, the diameter of the nozzle outlet, and the angle of taper from nozzle inlet to nozzle outlet are varied between nozzles 418a, 418b, and 418c. Relatively larger diameter outlets (e.g., nozzle 418a) may result in less concentrated shock waves in the distal direction and/or bubbles than relatively smaller diameter outlets (e.g., 418b and/or 418c). In some examples, including a smaller nozzle outlet diameter may result in more sonic energy dampening by the nozzle and thus a relatively lower total sonic output, but a higher sonic output in the distal direction. The relative steepness of the angle of taper from nozzle inlet to outlet may also impact the manner in which shock waves and/or bubbles propagate from the nozzle outlet. Shock wave and/or bubble concentration may thus be tailored for a given treatment based on the diameter of the nozzle selected. FIG. 4B illustrates an exemplary catheter 400 that includes a catheter body 401, a plurality of shock wave emitters 404, 406, and 408, and a nozzle configured to direct shock waves distally of the nozzle outlet at an angle offset from a longitudinal axis 481 of the catheter body 481 (e.g., off-center). Such a nozzle configuration may be desirable, for instance, to treat nodular or eccentric calcifications in the vasculature with concentrated shock waves and/or bubbles emitted from the nozzle. In some examples, the nozzles herein are removably attached to a catheter (e.g., removably attached to catheter body 401). This may enable a physician to easily swap nozzles to utilize a nozzle having the required dimensions/other design parameters for a given procedure. In some examples, the nozzles may be friction fit, screwed, clamped, or otherwise removably fastened to the catheter. In other examples, the nozzles may be permanently affixed to the catheter body (e.g., by spot welding, laser welding, friction welding, or other permanent attachment method). In some examples, the nozzle may be integrally formed with the distal region of the catheter.

In some examples, the catheters and nozzles described herein are utilized for pacemaker lead removal. Cardiac tissue surrounding pacemaker leads inserted into the tissue can become scarred/calcified/fibrotic/multi-morphic over time, making it difficult to remove the leads. With the passage of time, the leads can become entrenched or fused with the neighboring tissues and blood vessels. The formation of fibrous tissue, referred to as adhesions, around the leads can exacerbate the challenge of extraction. Additionally, tissue ingrowth into the insulation of the lead can add further complexity to the removal process Shock wave treatment can break up the scarred/calcified/fibrotic/multi-morphic tissue, thus loosening the attachment between the tissue and the pacemaker leads and enabling easier removal of the leads. In some examples, the nozzles described herein may be configured to assist in pacemaker lead removal, for instance, by concentrating shockwaves on tissue attached to a pacemaker lead within the nozzle and at the nozzle outlet and/or by scraping tissue from the pacemaker lead using an edge of the nozzle as the pacemaker lead is inserted into the nozzle outlet.

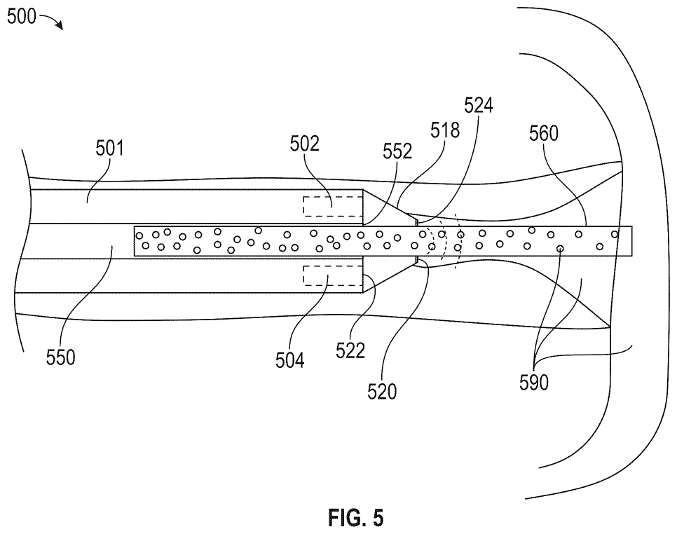

FIG. 5 illustrates an exemplary catheter 500 including shock wave emitters 502 and 504, a lumen 550 extending along the length of catheter body 510, and a nozzle 518 at the distal end of catheter body 501. The nozzle 518 and lumen 550 may be configured for removing pacemaker lead 560 from cardiac tissue 588 and fibrotic/calcified buildup 590. Nozzle 518 includes an inlet 522 and an outlet 520. The inlet may be positioned adjacent to an opening in catheter body 501 forming an inlet 552 to lumen 550. The nozzle outlet 520 and lumen inlet 552 may each be configured to receive pacemaker lead 560. For instance, the catheter 500 can be positioned within a vasculature such that pacemaker lead 560 is inserted into the nozzle inlet 520 and through the lumen inlet 552 into lumen 550. Pacemaker lead 560 may thus act as a guidewire for catheter 500, enabling a user to advance the catheter to a point of attachment between the pacemaker lead and cardiac tissue (e.g., heart muscle).

In some instances, fibrotic and/or calcified tissue can build up along the length of the pacemaker lead, making it difficult to advance a catheter along the lead to an attachment point at the heart tissue. Nozzle 518 may include a beveled edge at the outlet 520 such that as the catheter 500 is advanced over the pacemaker lead 560, the beveled edge scrapes fibrotic and/or calcified tissue away from the pacemaker lead. Nozzle 518 is also configured to concentrate shock waves and/or bubbles at the nozzle outlet 520. One or more shock waves may be generated using shock wave emitters 502 and/or 504 after a pacemaker lead has been inserted into the nozzle outlet 520. The shock waves concentrated at the nozzle outlet 520 may break up calcifications, multi-morphology tissue, and/or fibrotic tissue lodged to the pacemaker lead 560 at or near the nozzle outlet 520. The shock waves may then propagate distally from the nozzle outlet to break up fibrotic and/or calcified tissue located distally of the nozzle outlet 520 to further assist in releasing the pacemaker lead.

If the one or more shock waves do not free the pacemaker lead from the tissue, the catheter 500 can optionally be advanced further along the pacemaker lead 560 toward the calcified/fibrotic tissue 590 and/or a second plurality of shock waves can be generated. This process can be repeated as needed until the attachment between the pacemaker lead and cardiac tissue is sufficiently loosened such that the pacemaker lead can be removed either by removing the catheter along with the pacemaker lead, or by removing the catheter and inserting a separate tool for pacemaker lead removal.

FIG. 6 illustrates an exemplary method for generating shock waves to release a pacemaker lead from calcified/fibrotic/multimorphic tissue as described above. At block 602, a pacemaker lead is inserted into a lumen of a catheter via an opening in a nozzle provided at the distal end of the catheter. The pacemaker lead may be inserted into the nozzle outlet and directed from the nozzle outlet to an inlet of a lumen in the catheter body. At block 604, the catheter is advanced along the pacemaker lead to a target treatment site comprising fibrotic tissue. As the nozzle is advanced along the pacemaker lead to the target treatment site, the nozzle may scrape or otherwise dislodge fibrotic and/or calcified tissue from the pacemaker lead using a beveled edge provided at the outlet of the nozzle. At block 606, one or more shock waves are generated using at least one shock wave emitter included on the catheter to at least partially break up fibrotic tissue so that the pacemaker lead can be removed. The shock waves may break up calcified/fibrotic tissue on a portion of the pacemaker lead that has been inserted into the nozzle at or near the nozzle outlet (e.g., within the nozzle) and/or distally of the nozzle outlet as the shock waves propagate distally from the nozzle outlet. Blocks 604 and 606 may optionally be iteratively repeated until the pacemaker lead can be removed.

In some examples, the catheters described herein may be utilized to break up calcifications, multi-morphology tissue, and/or fibrotic tissue in instances where no pacemaker lead is inserted into the catheter. FIG. 7 illustrates an exemplary method for generating shock waves using a catheter that includes a nozzle for breaking up calcified or fibrotic tissue. At block 702, a distal portion of a catheter is positioned adjacent to an occlusion or other lesion in a vessel. At block 704, one or more shock waves are emitted from one or more shock wave emitters located at the distal portion of the catheter such that the shock waves propagate in a distal direction.

At block 706, the one or more distally propagating shock waves are directed by a nozzle located at a distal end of the catheter toward an outlet of the nozzle. The shock waves may be concentrated together as they are directed toward the outlet of the nozzle due to a convergent configuration of the nozzle. In some examples, at least one bubble may also be generated during shock wave generation by the one or more shock wave emitters. The at least one bubble may be directed toward an outlet of the nozzle. Directing the at least one bubble toward the outlet of the nozzle may cause the at least one bubble to concentrate at the outlet similarly to the shock waves. A peak concentration of the shock waves and/or bubbles may be reached at the outlet of the nozzle.

At block 708, the concentrated one or more shock waves and/or at least one bubble may be directed from the outlet of the nozzle toward the occlusion or other lesion to break up calcified, mutli-morphology, or fibrotic tissue at the occlusion or other lesion. In some examples, a fluid is supplied to the nozzle inlet to replace fluid that exits the outlet of the nozzle when emitting the one or more shock waves using a fluid supply line that extends along the length of the catheter body to the nozzle inlet. In some examples, debris may be removed from the body lumen via the outlet of the nozzle using a fluid return line of the catheter that extends from an inlet of the nozzle along the length of the catheter body. The catheter may optionally then be advanced further into the vessel and one or more additional shock waves may be emitted from the one or more shock wave emitters so that the shock waves concentrate at the outlet of the nozzle and propagate distally of the catheter body via the outlet of the nozzle.

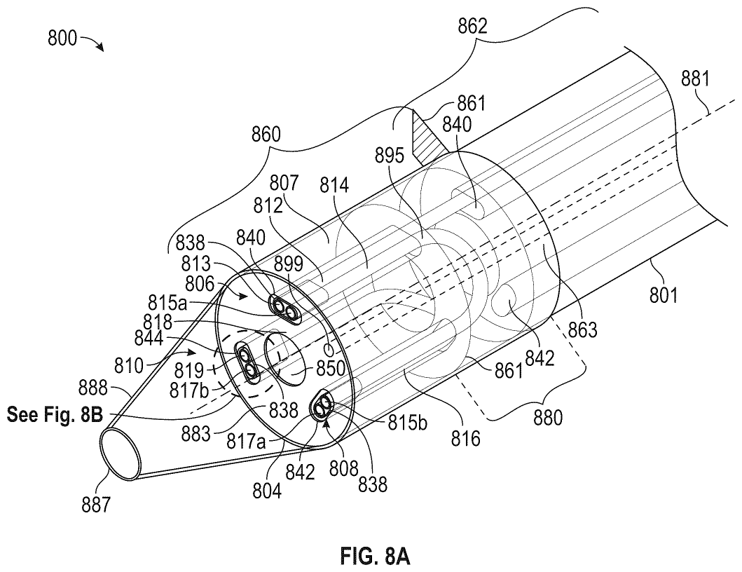

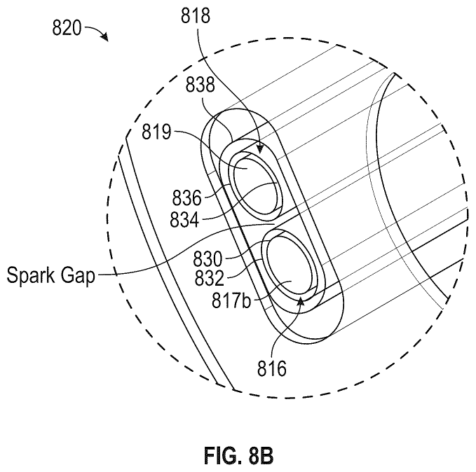

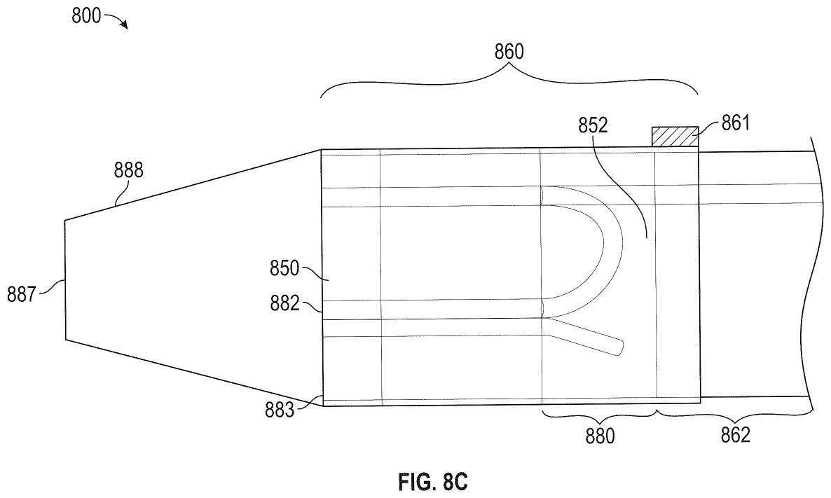

The nozzles described above may be utilized in combination with a variety of forward-firing shock wave emitter configurations. Below, FIGS. 8A-13B illustrate exemplary catheter and shock wave emitter configurations that may be utilized in combination with the nozzles described herein to direct concentrated distally propagating shock waves toward a target treatment region.

FIG. 8A illustrates an isometric view of an example of a distal end 804 of an exemplary catheter 800, which can be used for catheter 10 of FIG. 1. The catheter 800 of FIG. 8A includes a catheter body 801 with a distal end 804. A plurality of shock wave emitters 806, 808, and 810 (shown more clearly in the detailed view of FIG. 8B) are positioned at the distal end 804 of the catheter body 801. Each shock wave emitter 806-810 is configured to generate a shock wave that propagates distally of the catheter body 801 (i.e., distally of distal end 804). A nozzle 888 is positioned distally of the shock wave emitters 806-810 and configured such that shock waves emitted by the shock wave emitters 806-810 are directed into the nozzle toward an outlet of the nozzle 220. The nozzle 888 may include any of the features of the nozzles described above (e.g., nozzle 18, nozzle 218, nozzle 418 and 418a-c, and nozzle 518). For instance, the nozzle 888 is configured such that shock waves and/or bubbles are concentrated at the nozzle outlet and directed from the nozzle outlet 887 distally of the nozzle toward a target treatment region. In the example depicted in FIG. 8A, the nozzle 888 is positioned such that its inlet 889 is positioned distally of shock wave emitters 806-810 and shock wave emitters 806-810 are positioned radially inward of the outer diameter of the nozzle inlet such that the shock waves are emitted into the interior of the nozzle 888 and directed to the nozzle outlet 887. However, in some examples at least a portion of the nozzle may be positioned proximally of the shock wave emitters on the catheter body and/or an inlet to the nozzle may be positioned radially inward of at least one shock wave emitter (e.g., as illustrated in FIGS. 9C-9D and 10A-10B).

In some embodiments, the catheter body 801 may have an outer diameter of between 3 Fr and 14 Fr (French Gauge). In some embodiments, the catheter body 801 may have an outer diameter of between 1 Fr and 20 Fr. In some embodiments, the catheter body 801 may have an outer diameter of between 1 Fr and 100 Fr. In some embodiments, the catheter body 801 may have an outer diameter of at least 1 Fr, at least 2 Fr, at least 3 Fr, at least 4 Fr, at least 5 Fr, at least 6 Fr, at least 7 Fr at least 8 Fr, at least 9 Fr, at least 10 Fr, at least 11 Fr, at least 12 Fr, at least 13 Fr, at least 14 Fr, at least 15 Fr, at least 16 Fr, at least 17 Fr, at least 18 Fr, at least 19 Fr, or at least 20 Fr.

In some embodiments, the catheter body may have an outer diameter of no more than 20 Fr, no more than 19 Fr, no more than 18 Fr, no more than 17 Fr, no more than 16 Fr, no more than 15 Fr, no more than 14 Fr, no more than 13 Fr, no more than 12 Fr, no more than 11 Fr, no more than 10 Fr, no more than 9 Fr, no more than 8 Fr, no more than 7 Fr, no more than 6 Fr, no more than 5 Fr, no more than 4 Fr, no more than 3 Fr, no more than 2 Fr, or no more than 1 Fr. In some examples, an outer diameter of the inlet 889 of nozzle 888 is the same as the outer diameter of the catheter body 801. In some examples, the outer diameter of the inlet 889 of nozzle 888 is narrower than or wider than the outer diameter of the catheter body 801.

In the illustrated embodiment, the shock wave emitters 806, 808, and 810 are evenly spaced (positioned at increments of about 120 degrees) about the longitudinal axis 881; however, a variety of different spacing configurations can be implemented without deviating from the scope of the disclosure. In some embodiments, the shock wave emitters may be spaced apart from one another by a distance of between 0.1 millimeters (mm) and 20 millimeters (mm). In some embodiments, the shock wave emitters may be spaced apart from one another by a distance of between 1 millimeters (mm) and 10 millimeters (mm). In some embodiments, the shock wave emitters may be spaced apart from one another by between 2 mm and 5 millimeters (mm). The shock wave emitters may be spaced apart from one another by at least 1 millimeters (mm), at least 2 millimeters (mm), at least 3 millimeters (mm), at least 4 millimeters (mm), at least 5 millimeters (mm), at least 6 millimeters (mm), at least 7 millimeters (mm), at least 8 millimeters (mm), at least 9 millimeters (mm), at least 10 millimeters (mm), at least 12 millimeters (mm), at least 13 millimeters (mm), at least 14 millimeters (mm), at least 15 millimeters (mm), at least 16 millimeters (mm), at least 17 millimeters (mm), at least 18 millimeters (mm), at least 19 millimeters (mm), or at least 20 millimeters (mm). The shock wave emitters may be spaced apart from one another by no more than 20 millimeters (mm), no more than 19 mm, no more than 18 millimeters (mm), no more than 17 millimeters (mm), no more than 16 millimeters (mm), no more than 15 millimeters (mm), no more than 14 millimeters (mm), no more than 13 millimeters (mm), no more than 12 millimeters (mm), no more than 11 millimeters (mm), no more than 10 millimeters (mm), no more than 9 millimeters (mm), no more than 8 millimeters (mm), no more than 7 millimeters (mm), no more than 6 millimeters (mm), no more than 5 millimeters (mm), no more than 4 millimeters (mm), no more than 3 millimeters (mm), no more than 2 millimeters (mm), or no more than 1 millimeters (mm). The emitters may be spaced apart from one another by a distance set to optimize the constructive interference of shock waves generated by the emitters (e.g., depending on sonic output from individual emitters, acoustic properties of the propagating medium, etc.) In some embodiments, the distance between shock wave emitters is the distance between the two center points of two respective electrode pairs. In some embodiments, the distance between shock wave emitters is measured as the distance between the center points of two respective emitter bands.

In the exemplary embodiment of FIG. 8A, a first shock wave emitter 806 of the plurality of shock wave emitters includes a distal tip 813 of a first insulated wire 812. The insulated wire 812 extends along the length of the catheter body 801 from the distal end 804 (e.g., so that it can be connected to a voltage source proximally of the distal end (for instance, at a proximal end of the catheter), as described further below). A second insulated wire 814 extends from the first shock wave emitter 806 to the second shock wave emitter 808. The second insulated wire includes a first exposed distal tip 815a forming an electrode pair with distal tip 813 separated by a spark gap, thus forming shock wave emitter 806, and a second exposed distal tip 815b forming part of an electrode pair at shock wave emitter 808, as described below. As used herein, an “exposed end,” “exposed tip,” and/or “exposed distal tip” of an insulated wire may refer to a portion of the wire from which the insulation has been removed, thus revealing a portion of the conductive wire. However, while the emitters herein are often described as including the exposed distal ends/tips of insulated wires, it should be understood that any suitable conductor may serve as an electrode of the emitters.

The second insulated wire 814 extends proximally from shock wave emitter 806 into the catheter body 801 for a first distance, and loops around, for instance as illustrated by the bend 895 forming the U-shaped portion of insulated wire 814, to extend distally toward shock wave emitter 808. A third insulated wire 816 includes a first exposed distal tip 817a at shock wave emitter 808. The second exposed distal tip 815b of second insulated wire 814 and first exposed distal tip 817b of the third insulated wire 816 form an electrode pair separated by a spark gap, thus forming shock wave emitter 808. The third insulated wire 816 wire extends from the second shock wave emitter 808 to a third shock wave emitter 810. Similar to the second insulated wire 814, the third insulated wire 816 extends proximally into the catheter body 101 for a first distance, and loops around to extend distally toward shock wave emitter 810. The third insulated wire 816 includes a second exposed distal tip 817b at shock wave emitter 810, forming an electrode pair with exposed distal tip 819 of a fourth insulated wire. The exposed distal tips 817b and 819 form an electrode pair separated by a spark gap, thus forming third shock wave emitter 810. The fourth insulated wire 818 extends proximally into the catheter body and along the length of the catheter body 801 from the distal end 804 to connect to a positive terminal of a voltage source. Accordingly, when a voltage is applied across the first insulated wire 812 connected to the negative terminal of the voltage source and the fourth insulated wire connected to the positive terminal of the voltage source, a plurality of shock waves are simultaneously generated as an electrical current traverses the spark gaps separating the exposed distal tips of each insulated wire at shock wave emitters 806-810.

In some embodiments, the shock wave emitters 806-810 of the catheter 800 shown in FIG. 8A are electrically connected in series such that an electrical pulse applied across insulated wires connected to a negative and positive terminal of a voltage source (such as wire 812 and 818), respectively, causes each of the plurality of shock wave emitters to emit a respective shock wave. In some embodiments, at least one first shock wave emitter of a plurality of shock wave emitters can be driven independently of at least a second shock wave emitter of the plurality of shock wave emitters. Accordingly, in some embodiments, rather than extending wires between all of the shock wave emitters such that applying a single voltage pulse causes each of the shock wave emitters to generate shock waves in series, one or more shock wave emitters can each include an electrode pair configured to generate shock waves independently of the other shock wave emitters. In some embodiments, the electrode pair at each shock wave emitter (e.g., shock wave emitters 806-810) can be formed of the exposed distal tips of a first and second wire that each extend along the length of catheter 800 from the distal end 804 to electrically couple to a respective positive and negative terminal (or to ground) of a voltage source (e.g., each shock wave emitter may be connected to a respective channel of a relay such that it can be driven independently of the other emitters). In such embodiments, when a voltage pulse is applied across the first and second wire of an independently driven shock wave emitter, a current flows from an exposed distal tip of the first insulated wire to the exposed distal tip of the second insulted wire to generate a shock wave, but that shock wave emitter is electrically isolated from the remaining shock wave emitters.

In some embodiments, catheter body 801 includes one or more lumens extending within the catheter body. In some embodiments, one or more of the insulated wires (e.g., wires 812 and 818 in FIG. 8A) extend along the length of the catheter within a respective lumen to connect to a voltage source. As described above, other insulated wires (e.g., wires 814 and 816 of FIG. 8A) are routed between respective shock wave emitters to carry the current received from the voltage between each of the emitters. Accordingly, the wires routed between respective shock wave emitters may extend into a first lumen of the catheter body 801 in a first direction toward a first shock wave emitter and extend into a second lumen in a second direction toward a second shock wave emitter. As depicted in FIG. 8A, insulated wire 814 is formed into a U-shape, where the parallel portions of the U-shape respectively of extend into lumens 840 and 842. The bend 895 in the U-shape of wire 814 is formed within a cavity 880 formed between a surface 861 of a first section 860 of catheter body 801 and second surface 863 of a second section 862 of the catheter body 801 near the distal end 804. In some embodiments, lumens 840, 842, and/or 844 extend from respective orifices in the distal most surface of catheter 800 at distal end 804 into section 860 of the catheter body 801 to a respective orifice of surface 861 facing cavity 880. In some embodiments, any of the respective lumens 840, 842, and/or 844 extend into the second section 862 of catheter body 801 from a respective orifice of surface 863 on the opposite side of cavity 880 along the same respective longitudinal axes as in section 862.

In some embodiments, cavity 880 is formed into section 860 of catheter body 801. Section 860 may be a removable tip that can be friction fit onto section 862. Cavity 880 may be a hollow portion of section 860 that is configured such that a portion 861 of section 862 can extend into the cavity 880 when section 860 is friction fit with section 862 (e.g., such that sections 860 and 862 overlap with one another when section 860 is friction fit to section 862). The removability of section 860 from section 862 can allow for placement/replacement of wires and/or other device maintenance.

In some embodiments, the catheter 800 includes a central lumen 850 extending from the distal end 804 of the catheter along the length of the catheter body 801. In some embodiments the central lumen 850 may be enclosed within a tube 852 that extends from section 860 to section 862 through cavity 880 within the catheter body 801, as shown in FIG. 8C. In some embodiments, the central lumen 850 extends from an orifice at the distal end 804 to an orifice at the opposite end of catheter body 801. The central lumen 850 may be configured to receive a guide wire. For instance, the guide wire can be inserted into the catheter 800 via central lumen 850 proximally of the distal end 804 and exit the catheter via the nozzle outlet 887 after exiting the central lumen 850 at the distal end 804. The guide wire can be used to guide the catheter into place within a body lumen (e.g., blood vessel, urinary tract, or other organ).

In some embodiments, the central lumen 850 is configured to receive a pacemaker wire lead at the distal end 804 of the catheter body 801 after the pacemaker lead is received via outlet 887 of nozzle 888. Pacemaker leads can be difficult to remove due to dense calcification, multi-morphology, and/or fibrotic tissue build-up. This calcification build-up can make extraction more difficult for the physician and riskier for the patient. The catheters described herein can be used to first break-up these dense calcifications using the shock waves generated by the plurality of shock wave emitters before removing the pacemaker lead. Breaking up the calcifications prior to removing the leads can lead to dramatic reduction in removal time.

In some embodiments, the catheter body 801 includes an aspiration lumen. In some embodiments, the aspiration lumen is for removing debris from a body lumen. In some embodiments central lumen 850 can be configured for aspiration. In some embodiments, the catheter body may include a separate lumen in addition to the central lumen 850 for aspiration. In some embodiments, the catheter 800 includes a marker band for determining an orientation of the catheter within a body lumen. Catheter 800 may include a fluid lumen 899. The fluid lumen 899 may serve as a fluid supply line and/or a fluid return line. The fluid lumen 899 may extend along the catheter body 801 from nozzle inlet 889 to a proximal end of the catheter body (e.g., to connect to a fluid source). The fluid lumen 899 may be used to replenish conductive fluid that the ejected from the nozzle 888 during shock wave treatment.

FIG. 8B illustrates a detail view of shock wave emitter 810, in accordance with some embodiments. Shock wave emitter 810 includes insulated wire 816 and insulated wire 818 described above with reference to FIG. 8A. Insulated wire 816 includes a conductive wire 830 disposed within an insulating tube 832. In some embodiments, the conductive wire 830 is a copper wire and insulating tube 832 is a polyimide tube. Similarly, insulated wire 818 includes a conductive wire 834 disposed within an insulating tube 836. In some embodiments, the conductive wire 834 is a Molybdenum wire and the insulating tube 836 is also a polyimide tube. In some embodiments, at least a portion of both of the insulating tubes 832 and 836 are disposed within a single outer insulating tube 838. In some embodiments, the outer insulating tube 838 is also a polyimide tube that provides an additional layer of insulation. The outer insulating tube 838 may separate the two insulated wires 816 and 818 from one another (e.g., by surrounding a portion of each of the insulated wires individually), and in turn separate their respective exposed distal tips (817b and 819) by a spark gap. An outer insulating tube 838 may be positioned at each of shock wave emitters 806, 808, and 810 to provide an additional layer of insulation and to maintain a spark gap between the exposed distal tips (e.g., 817a, 815b, 815a, and 813). As described above, the distal tips 817b and 819 of conductive wires 830 and 834, respectively, are exposed at the distal end of the catheter 800 to allow for generation of shock waves. The exposed distal tips 817b and 819 form an electrode pair separated by a spark gap such that when a voltage is applied across the conductive wires 830 and 834, a current flows from exposed distal tip 817b to the exposed distal tip 819 to generate a shock wave.

In some embodiments, the shock wave emitters 806-810 are arrayed symmetrically about the longitudinal axis 881 of the catheter body, for instance, as shown in FIG. 8A. In some embodiments, the shock wave emitters are instead arrayed asymmetrically about the longitudinal axis of the catheter body. For example, two of the shock wave emitters may be positioned more closely to one another than a third shock wave emitter. Symmetric arrangement of the shock wave emitters about the longitudinal axis may be desirable for optimizing constructive interference between all of the shock wave emitters. However, arranging the shock wave emitters asymmetrically about the axis can provide for asymmetric shock waves, which may be beneficial, for instance, if an occlusion is concentrated at various locations within a body lumen relative to the distal end 804 of the catheter 800 (i.e., if the occlusion is more concentrated at various location about the circumference of the catheter 800).

In some embodiments, a distal most surface 882 of one or more shock wave emitters of the plurality of shock wave emitters is flush with a distal most surface 883 of the distal end of the catheter body (which may be at least partially located within an interior portion of nozzle 888), for instance, as shown in FIG. 8C. In some embodiments, a distal most surface 882 of a shock wave emitter of the plurality of shock wave emitters is instead positioned forward from a distal most surface 883 of the distal end of the catheter body 801. Alternatively, the distal most surface of any of the shock wave emitters may be recessed from a distal most surface of the distal end of the catheter body.

In some embodiments, the plurality of shock wave emitters (e.g., 806-810) are arrayed about a longitudinal axis of the catheter body at the same distal location relative to the distal end of the catheter body, for instance, as shown in FIG. 8C. In some embodiments, a first shock wave emitter of the plurality of shock wave emitters is positioned at a first location relative to the distal end of the catheter body and a second shock wave emitter of the plurality of shock wave emitters is positioned at a second location relative to the distal end of the catheter body. For example, a first shock wave emitter may be positioned such that its distal most surface is flush with the distal end 804 of the catheter body 801, one or more of the shock wave emitters may be positioned distally relative to the first shock wave emitter, and one or more shock wave emitters may be placed proximally relative to the first shock wave emitter. Alternatively, a first shock wave emitter may be positioned such that its distal most surface is flush with the distal end 804 of the catheter body 801, a second shock wave emitter may be positioned proximally relative to the first shock wave emitter, and a third shock wave emitter may be positioned proximally relative to the second shock wave emitter. It should be understood that the aforementioned configurations are meant to be exemplary, and the shock wave emitters could be positioned in a variety of different locations relative to one another without deviating from the scope of this disclosure. A variety of different arrangements of the shock wave emitters that may be included on catheter 800 are shown below in FIGS. 9A-9D. Additionally, it should be understood that while catheter 800 is described as including three shock wave emitters, in some embodiments, the catheters described herein may include additional shock wave emitters (i.e., more than the three provided on catheter 800) positioned at the distal end of the catheter body. The additional shock wave emitters may be included, for instance, to generate a more powerful shock wave for treating occlusions or otherwise breaking up calculi within a human body.

FIG. 9A illustrates a front view of a catheter 900a, in accordance with some embodiments. Catheter 900a includes a plurality of shock wave emitters 906a-909a arrayed asymmetrically about longitudinal axis 981 of catheter 900a. An outer surface of each of shock wave emitters 906a-909a is approximately flush with an inner diameter of an inlet 922a to a nozzle 918a may be positioned approximately flush with catheter body 401a. Shock waves emitted from shock wave emitters 906a-909a are directed toward outlet 920a of nozzle 918a as described throughout. FIG. 9B illustrates a front view of a catheter 900b, in accordance with some embodiments. Catheter 900b similarly includes a plurality of shock wave emitters 906b-908b also arrayed asymmetrically about longitudinal axis 981 of catheter 900b. Shock wave emitter 906b is positioned such that its outer circumferential surface is positioned externally relative an outer circumferential surface of the catheter body 901b, shock wave emitter 907b is positioned such that its outer circumferential surface is positioned inset relative an outer circumferential surface of the catheter body 901b, and shock wave emitter 908b is positioned such that its outer circumferential surface is flush with an outer circumferential surface of the catheter body 901b. Each of shock wave emitters 906b, 907b, and 908b are positioned radially interior to an inlet 922b of nozzle 918b. The diameter of the inlet 922b of nozzle 918b is larger than the outer diameter of catheter body 401b and shock wave emitter 406b is positioned radially interior to inlet 922b. Shock waves emitted from shock wave emitters 906b-909b are directed toward outlet 920b of nozzle 918b as described throughout.

FIGS. 9C-9D illustrate a side view of catheters 900c-900e, respectively, in accordance with some embodiments. Catheter 900c depicted in FIG. 9C includes a plurality of shock wave emitters 906c-908c at a distal end 904 of the catheter body 901. Shock wave emitters 906c-908c are positioned such that a distal most surface of each of shock wave emitters 906c-908c is positioned forward of a distal most surface of the distal end 904 of the catheter body 901 and at least partially within a nozzle 918 positioned distally of the distal end to catheter body 901. All of shock wave emitters 906c-908c are further positioned at the same distal location relative to the distal most surface of the catheter body 901. Shock wave emitters 906c-908c may be arrayed symmetrically or asymmetrically about the longitudinal axis 981 of the catheter body 901. Nozzle 918 is configured such that shock waves emitted from emitters 906c-908c propagate from the inlet 922 of nozzle 918 toward an outlet 920 of nozzle 918 as described throughout.

Catheter 900d of FIG. 9D includes a plurality of shock wave emitters 906d-908d at the distal end of catheter body 901. Shock wave emitters 906d-908d are positioned such that a distal most surface of each of shock wave emitters 906d and 908d is forward of the distal most surface of catheter body 901 and shock wave emitter 907d is positioned such that a distal most surface of shock wave emitter 907d is recessed from a distal most surface of the catheter body 901. A nozzle 918 is positioned at the distal end of catheter 900d and is configured such that shock waves emitted from emitters 906d-908d propagate from the inlet 922 of nozzle 918 toward an outlet 920 of nozzle 918 as described throughout.

In some examples (e.g., the example illustrated in FIG. 9B described above), one or more shock wave emitters may be positioned radially exterior of an outer surface of a catheter body. For instance, in some examples a conductive emitter band may be positioned in a cavity of an outer surface of the catheter body and configured such that at least a portion of the conductive emitter band is positioned radial exterior to an outer surface of the catheter body, as shown in FIGS. 10A and 10B described below. In some embodiments, a nozzle may be configured to at least partially overlap with/circumscribe shock wave emitters positioned radially exterior to the catheter body such that shock waves emitted from the shock wave emitters are directed by the portion of the nozzle enclosing the emitters toward an outlet of the nozzle.

With respect to catheter 800 described above, the shock wave emitters are configured to respectively emit shock waves by creating a spark across a spark gap formed between the exposed distal ends of two wires. In some embodiments, such as the exemplary embodiment depicted in FIGS. 10A and 10B, the shock wave emitters may include conductive emitter bands. Shock waves may be generated by creating a spark across a spark gap between an exposed tip of a wire and a conductive emitter band. In some embodiments, the conductive emitter bands are conductive cylindrical tubes that at least partially circumscribe wires used to either generate shock waves and/or transfer current between multiple conductive emitter bands. Accordingly, in some embodiments, the shock wave emitters of the catheters described herein may include a respective conductive emitter band, a first insulated wire with an exposed distal tip positioned such that a spark gap is formed between the exposed distal tip and the conductive emitter band, and a second insulated wire that is electrically connected (e.g., soldered) to the conductive emitter band. The connected wire can be routed to a next shock wave emitter to transfer the electrical current from the first conductive emitter band to an electrode of the next shock wave emitter.

FIGS. 10A and 10B illustrate an exemplary catheter 1000 for generating shock waves in a forward direction using shock wave emitters formed by conductive emitter bands separated by a spark gap from an electrode. Catheter 1000 includes a catheter body 1001 having a distal end 1004. The catheter 1000, as shown, includes a plurality of shock wave emitters 1006-1010 disposed at the distal end 1004 of the catheter body 1001. A nozzle 1088 is positioned at the distal end of catheter 1000 and configured to direct shock waves from the shock wave emitters 1006-1010 toward a nozzle outlet 1087. The nozzle 1088 may include any of the features of the nozzles described herein (e.g., as described above with reference to nozzle 218, 418, 418a-418c, 518, 888, etc.).

The nozzle 1088 is positioned such that an inlet region 1089 of the nozzle 1088 at least partially overlaps with shock wave emitters 1006-1010 with respect to a longitudinal axis of the catheter body. The inlet region of nozzle 1088 includes a first portion that tapers away from the catheter body 1001 in the distal direction (e.g., referred to herein as a diverging portion 1089a). The diverging portion 1089a is positioned at least partially proximally of the shock wave emitters 1006-1010. The inlet region of nozzle 1088 also includes a second portion that extends straight along the catheter body 1001 (e.g., referred to herein as a straight portion 1089b). The straight portion at least partially overlaps with shock wave emitters 1006-1010 with respect to a longitudinal axis of the catheter body and is positioned between the diverging region 1089a of inlet region 1089 and a converging portion 1086 of nozzle 1088 that extends from the straight portion 1089b to the nozzle outlet 1087. The nozzle 1088 is configured to partially enclose shock wave emitters 1006-1010 proximally of the distal end of the catheter body such that radially and proximally propagating shock waves are redirected toward the nozzle outlet 1087 via the converging portion 1086.

Each shock wave emitter 1006-1010 can be configured to generate a shock wave that propagates distally of the catheter body 1001 (i.e., distally of distal end 1004 via nozzle outlet 1087). The shock wave emitters 1006-1010 can be arrayed about a longitudinal axis of the catheter body 1001 and configured such that shock waves emitted from the plurality of shock wave emitters 1006-1010 constructively interfere distally of the catheter body 1001 within nozzle 1088 and distally of the nozzle outlet 1087. The shock waves and/or bubbles produced during shock wave generation may be concentrated at the nozzle outlet 1087 and propagate distally of the nozzle outlet 1087 toward a treatment region. Thus, the positioning of the shock wave emitters 1006-1010 and use of nozzle 1088 to concentrate the shock waves generated by emitters 1006-1010 can maximize the shock wave intensity distally of the catheter body by causing shock waves emitted by each respective emitter to combine with one another to produce an amplified combined shock wave.

FIGS. 10A and 10B illustrate an isometric view of the distal end 1004 of catheter 1000 and a side view of the distal end of catheter 1000, respectively. In some embodiments, the shock wave emitters 1006-1010 of the catheter 1000 shown in FIGS. 10A and 10B are electrically connected in series. Shock wave emitter 1006 includes a conductive band 1070 with a distal end and a proximal end, the distal end of the shock wave emitter 1006 positioned relatively closer to the distal end 1004 of the catheter body 1001. The conductive band 1070 may form an electrode pair with an exposed end 1013 of a first insulated wire 1012, the exposed end 1013 positioned at the distal end of conductive band 1070. The insulated wire 1012 may be positioned within the conductive band 1070 and separated by a spark gap from the conductive band 1070 for generating shock waves when a voltage is applied across the spark gap. In some embodiments, the insulated wire 1012 extends along the length of catheter body 1001 from within the conductive band 1070 at the distal end 1004 such that the first insulated wire 1012 can be connected to a voltage source.

In some embodiments, shock wave emitter 1006 includes a second insulated wire 1014 with a first exposed end 1015a electrically connected (e.g., may be soldered, crimped, taped, adhered, clamped, or otherwise electrically connected to) to the conductive emitter band 1070. In some embodiments, the insulated wire 1014 is disposed at least partially within the interior of conductive emitter band 1070 and the exposed end 1015a is electrically connected (e.g., may be soldered, crimped, taped, clamped, or otherwise connected to) to an inner surface of the conductive band 1070. It should be understood, however, that the exposed end 1015a could be electrically connected to any conductive surface of the conductive band 1070. In some embodiments, a second exposed end (not shown) of the second insulated wire 1014 is electrically connected to a conductive band 1072 of a second shock wave emitter 1007 to transfer the voltage between the conductive band 1070 of the first shock wave emitter 1006 and the conductive band 1072 of the second shock wave emitter 1007. Similar to conductive band 1070, the conductive band 1072 has a distal end and a proximal end, the distal end of the conductive band 1072 positioned relatively closer to the distal end 1004 of the catheter body 1001. Shock wave emitter 1007 includes a first exposed tip 1017a of a third insulated wire 1016 positioned at least partially within the interior of conductive emitter band 1072, the exposed tip 1071a positioned at the distal end of the conductive emitter band 1072 such that shock waves generated by the shock wave emitter 1007 propagate distally. Insulated wire 1016 is positioned to form a spark gap between the exposed tip 1017a and the conductive emitter band 1072 such that an electrical current can flow between the conductive emitter band 1072 and the exposed tip 1017a to generate a shock wave distally of the distal end of the catheter body 1001 within nozzle 1088 that propagates distally of the nozzle outlet 1087.

Insulated wire 1016 is also positioned to transfer current from the second shock wave emitter 1007 to a third shock wave emitter 1008, and a spark gap is formed between a second exposed end 1017b of insulated wire 1016 and a conductive band 1074 at the distal end of the conductive emitter band, as described below. Specifically, insulated wire 1016 is inserted into both conductive bands 1072 and 1074 from the proximal end of each conductive band such that a portion of insulated wire 1016 extends into both conductive bands 1072 and 1074 toward the distal end of each respective conductive emitter band. Insulated wire 1016 includes a second exposed end 1017b disposed at the distal end of the conductive emitter 1074. Insulated wire 1016 is positioned such that a spark gap separates the second exposed end 1017b from the conductive band 1074. Accordingly, when an electrical current flows between exposed end 1017b and conductive band 1074 another shock wave is generated at shock wave emitter 1008 and propagates distally of the shock wave emitter 1008.

In some embodiments, a first exposed end 1019a of a fourth insulated wire 1018 is electrically connected (e.g., may be soldered, crimped, tapes, clamped, or otherwise electrically connected to) to the conductive band 1074. In some embodiments, the insulated wire 1018 is positioned at least partially within the interior of conductive band 1074 and the exposed end 1019a is electrically connected (e.g., may be soldered, crimped, tapes, clamped, or otherwise electrically connected to) to an inner surface of the conductive band. As with shock wave emitters 1006 and 1007, it should be understood that the exposed end 1019a could be electrically connected to any conductive surface of the conductive band 1074. In some embodiments, a second exposed end 1019b of the second insulated wire 1018 is electrically connected to a conductive band 1076 of a fourth shock wave emitter 1009 to transfer the electrical current between the conductive band 1074 of the third shock wave emitter 1008 and the conductive band 1076 of the fourth shock wave emitter 1009. More specifically, similar to conductive band 1070, 1072, and 1074, the conductive band 1076 has a distal end and a proximal end, the distal end positioned relatively closer to the distal end 1004 of the catheter body 1001. Insulated wire 1018 may extend outwardly from the distal end of the conductive emitter band 1074 and exit the conductive emitter band 1074 from its distal end. Insulated wire 1018 may then be directed toward the next shock wave emitter in the series, shock wave emitter 1009. Insulated wire 1018 may extend into the distal end of conductive band 1076 and a second exposed end 1019b of insulated wire 1018 may be electrically connected to the conductive band 1076 to transfer an electrical current between the conductive band 1074 of the third shock wave emitter 1008 and the conductive band 1076 of the fourth shock wave emitter 1009.

Shock wave emitter 1009 additionally includes a first exposed end 1021a of a fifth insulated wire 1020. The fifth insulated wire is disposed at least partially within the interior of conductive band 1076, and the first exposed end 1021a is disposed at the distal end of the conductive emitter band 1076. Insulated wire 1020 is positioned within conductive emitter band 1076 such that the exposed end 1021a is separated by a spark gap from the conductive emitter band 1076. Accordingly, when a current flows across the spark gap between the conductive emitter band 1076 and the exposed end 1021a a spark is created thus generating a shock wave that propagates distally of the distal end of the catheter body 1001 via nozzle outlet 1087.

Similar to insulated wire 1016, in some embodiments, the insulated wire 1020 is routed to a fifth shock wave emitter 1010 and an exposed end 1021b of wire 1020 is separated by a spark gap from a conductive band 1078 at the distal end of the conductive emitter band, as described in more detail below. Insulated wire 1020 may be inserted into both conductive bands 1076 and 1078 from the proximal end of each respective conductive band. A portion of insulated wire 1020 extends into both conductive bands 1076 and 1078 toward the distal end of each respective conductive emitter band, for instance, as shown in the side view of catheter 1000 in FIG. 10B. Insulated wire 1020 may include a second exposed end 1021b positioned at the distal end of the conductive emitter band 1078 (i.e., the end of the conductive emitter band facing the distal end of catheter body 1001), as described above. The insulated wire 1020 may be positioned such that the exposed end 1021b is separated by a spark gap from the conductive emitter band 1078. Accordingly, when the current flows between the exposed tip 1021b to conductive emitter band 1078 a shock wave is generated by shock wave emitter 1010. A sixth insulated wire 1022 with an exposed tip 1023 may be electrically connected (e.g., soldered crimped, tapes, clamped, or otherwise electrically connected to) to the conductive emitter band 1078 and may extend along the length of the catheter body 1001 from the conductive emitter band 1078 at the distal end 1004 to connect to a positive terminal of a voltage source. Accordingly, when a voltage is applied across the negative and positive terminal, and thus across wires 1012 and 1022, a plurality of shock waves are generated at each of the shock wave emitters 1006-1010.

In some embodiments, each of the respective shock wave emitters 1006-1010 are positioned within a respective cavity 1040 formed into the outer circumferential surface of catheter body 1001. In some embodiments, each cavity 1040 has a semi-circular shape sized such that a respective conductive emitter 1070-1078 band having a cylindrical shape can be positioned at least partially within a semi-circular cavity 1040. In some embodiments, each cavity 1040 extends along the length of catheter body 1001 from distal end 1004 (e.g., to a proximal end of the catheter body 1001). In some embodiments, one or more of the insulated wires included in shock wave emitters 1006-1010 extend within a respective cavity 1040 along the length of the catheter body 1001 to connect to a voltage source.

In some embodiments, the catheter 1000 includes a central lumen 1050 extending from the distal end 1004 along the length of the catheter body 1001. The central lumen may be aligned (e.g., concentric) with nozzle outlet 1087. The central lumen 1050 and nozzle outlet 1087 may be configured to receive a guide wire. For instance, the guide wire can be inserted into the catheter 1000 via central lumen 1050 proximally of the distal end 1004 and exit the catheter via the central lumen 1050 at the distal end 1004. The guide wire may extend through nozzle 1088 and exit the nozzle via outlet 1087. The guide wire can be used to guide the catheter into place within a body lumen (e.g., blood vessel or other organ). In some embodiments, the central lumen 1050, like central lumen 850 of catheter 800, is configured to receive a pacemaker wire lead at the distal end 1004 of the catheter body 1001 after the pacemaker lead is received via nozzle outlet 1087 for removing the pacemaker wire lead from a tissue, such as cardiac tissue. In some embodiments, the catheter body 1001 includes an aspiration lumen. In some embodiments, the aspiration lumen is for removing debris from a body lumen. In some embodiments central lumen 1050 can be configured for aspiration. In some embodiments, the catheter body may include a separate lumen (not shown) for aspiration. In some embodiments, the catheter 100 includes a marker band for determining an orientation of the catheter within a body lumen.

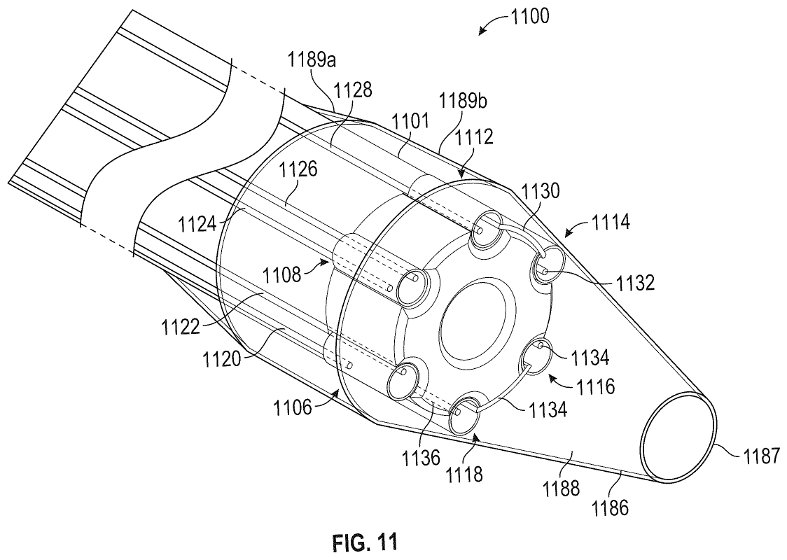

Although catheter 1000 is described as having five shock wave emitters electrically connected in series such that an electrical pulse applied to a first shock wave emitter of the plurality of shock wave emitters causes each of the plurality of shock wave emitters to emit a respective shock wave, it should be understood that the shock wave emitters could be configured such that any of the shock wave emitters of the plurality of shock wave emitters 1006-1010 can be driven independently of any of the other shock wave emitter of the plurality of shock wave emitters. An exemplary embodiment illustrating a catheter including a first set of shock wave emitters configured to be driven independently of one another and a second set of shock wave emitters configured to be driven in series is illustrated below in FIG. 11.

FIG. 11 illustrates an exemplary catheter 1100 including shock wave emitters 1106 and 1108 configured to fire independently of one another and of the other shock wave emitters 1112, 1114, 1116, and 1118 included on catheter 1100. Catheter 1100 of FIG. 11 also includes nozzle 1188 which may include the same features described above with reference to catheter 1000 of FIGS. 10A and 10B. Nozzle 1188 is positioned at the distal end of catheter 1100 and configured to direct shock waves from the shock wave emitters 1106-1118 toward a nozzle outlet 1087.

The nozzle 1188 is positioned such that an inlet region 1189 of the nozzle 1188 at least partially overlaps with shock wave emitters 1106-1118 with respect to a longitudinal axis of the catheter body. The inlet region of nozzle 1188 includes a first portion that tapers radially outwardly from the catheter body 1101 (e.g., referred to herein as a diverging portion 1189a).

The diverging portion 1189a is positioned at least partially proximally of the shock wave emitters 1106-1118 with respect to a longitudinal axis of the catheter bod. The inlet region of nozzle 1188 also includes a second portion that extends straight along the catheter body 1101 (e.g., referred to herein as a straight portion 1189b). The straight portion at least partially overlaps with shock wave emitters 1106-1118with respect to a longitudinal axis of the catheter bod and is positioned between the diverging region 1189a of inlet region 1189 and a converging portion 1186 of nozzle 1188 that extends from the straight portion 1189b to the nozzle outlet 1187. The nozzle 1188 is configured to partially enclose shock wave emitters 1106-1118 proximally of the distal end of the catheter body such that radially and proximally propagating shock waves are redirected toward the nozzle outlet 1187 via the converging portion 1186.

Shock wave emitter 1106 includes an electrode pair formed by an exposed end of an insulated wire 1122 and a conductive band 1170. The insulated wire 1122 extends into the conductive band 1170 from a proximal end of the conductive band toward a distal end of the conductive band 1170 and is positioned such that the exposed end of the wire 1122 is separated by a spark gap from conductive band 1170. The insulated wire 1122 may extend from the proximal end of the conductive band 1170 along the length of the catheter body 1101 of catheter 1100 to connect to a first terminal of a voltage source. A second insulated wire 1120 may extend into the conductive band 1170 from the proximal end of the band and connect to (e.g., may be soldered, crimped, tapes, clamped, or otherwise connected to) a surface of the conductive band 1170. The second insulated wire may extend from the proximal end of the conductive band 1170 along the length of the catheter body to connect to a second terminal of the voltage source. The insulated wires 1120 and 1122, respectively, may be connected to a negative and positive terminal of the voltage source. Accordingly, when a voltage is applied across wire 1120 and 1122, a shock wave is generated at shock wave emitter 1106, but the voltage applied across wire 1120 and 1122 does not result in shock waves at any of the other shock wave emitters provided on catheter 1100.

Similarly, shock wave emitter 1108 includes an electrode pair formed by an exposed end of an insulated wire 1126 and a conductive band 1172. The insulated wire 1126 extends into the conductive band 1172 from a proximal end of the conductive band toward a distal end of the conductive band 1172 and is positioned such that the exposed end of the wire 1126 is separated by a spark gap from conductive band 1172. The insulated wire 1126 may extend from the proximal end of the conductive band 1172 along the length of the catheter body 1101 of catheter 1100 to connect to a first terminal of a voltage source. A second insulated wire 1124 may extend into the conductive band 1172 from the proximal end of the band and connect to (e.g., may be soldered, crimped, tapes, clamped, or otherwise connected to) a surface of the conductive band 1172. The second insulated wire may extend from the proximal end of the conductive band 1172 along the length of the catheter body to connect to a second terminal of the voltage source. The insulated wires 1126 and 1124, respectively, may be connected to a negative and positive terminal of the voltage source. Accordingly, when a voltage is applied across wire 1124 and 1126, a shock wave is generated at shock wave emitter 1108, but the voltage applied across wire 1124 and 1126 does not result in shock waves at any of the other shock wave emitters provided on catheter 1100. In contrast, the plurality of shock wave emitters 1112, 1114, 1116, and 1118 are electrically connected in series and thus fire simultaneously with one another.

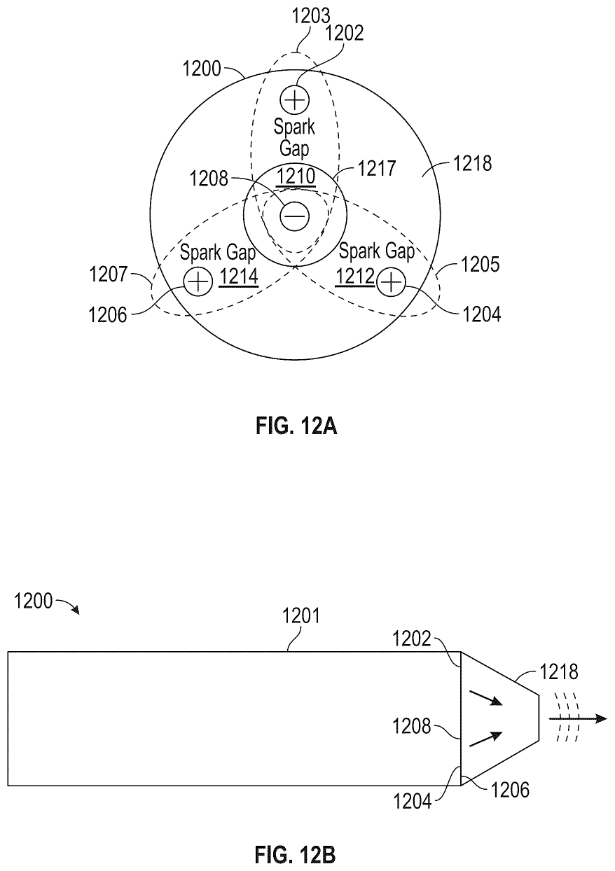

In some examples, it may be desirable to arrange the shock wave emitters and corresponding electrodes such that when the shock wave emitters of a catheter are simultaneously driven, a single cavitation bubble is formed that propagates distally of the distal end of the shock wave emitters toward a nozzle outlet (e.g., in contrast to a plurality of relatively smaller cavitation bubbles that may constructively interfere distally of the distal end). This may be accomplished, for instance, as described below with reference to FIGS. 12A and 12B, by configuring the shock wave emitters such that each of the shock wave emitters shares a common electrode. The common electrode may be positioned at a location between each of the other electrodes (e.g., the common electrode may be positioned at a center of a distal end of the catheter and an electrode of each respective shock wave emitter may be positioned at an equal distance from the common electrode about a circumference of the distal end of the catheter). Shock waves and/or bubble(s) generated by the emitters sharing a common electrode may be directed to and concentrated at a nozzle outlet as described throughout.

FIG. 12A illustrates a front view of an exemplary catheter 1200 for generating shock waves according to some embodiments. Catheter 1200 may include a plurality of shock wave emitters 1201, 1203, and 1205 positioned proximally of a nozzle 1218 located at the distal end of catheter body 1201. Each of the shock wave emitters 1201, 1203, and 1205 may include a respective electrode 1202, 1204, and 1206 connected to a respective positive terminal (e.g., supply terminal) of a voltage source (e.g., shock wave energy generator 530), for instance, by a supply wire that extends along the length of the catheter 1200. The respective electrodes 1202, 1204, and 1206 of each of shock wave emitters 1201, 1202, and 1203 may be spaced apart by a respective spark gap 1210, 1212, and 1214 from an electrode 1208 that is connected to a negative terminal (e.g., return terminal) of the voltage source. Accordingly, electrode 1208 may form one of the electrodes at each respective shock wave emitter. The respective positive terminals connected to each of electrodes 1202, 1204, and 1206 may be simultaneously pulsed, resulting in a voltage applied across the respective spark gaps 1210, 1212, and 1214 separating each of electrodes 1202, 1204, and 1206 from electrode 1208. The electrodes may be positioned such that when a sufficiently high voltage is applied across the electrodes 1202 and 1208, 1204 and 1208, and 1206 and 1208, each shock wave emitter 1201, 1203, and 1205 generates a shock wave that propagates in a direction forward of the distal end of the catheter body toward outlet 1217 of nozzle 1218. For instance, electrode 1208 may be positioned at an equal distance from each of electrodes 1202, 1204, and 1206. Electrode 1208 may be positioned at the center of the distal end of catheter 1200 aligned with nozzle outlet 1217, and electrodes 1202, 1204, and 1206 may be positioned at equal distances from electrode 1208 about the circumference of the distal end of the catheter 1200, as shown. Additionally, the shock wave emitters are configured such that when each simultaneously generates a shock wave, a single combined cavitation bubble may be formed rather than three separate cavitation bubbles that constructively interfere distally of the distal end of catheter 1200. The voltage polarity (i.e., direction of current flow) between the respective electrodes of shock wave emitters 1201, 1203, and 1205 may be switched between voltage pulses. Such polarity switching may promote more uniform wear of electrodes and extend device longevity. FIG. 12B illustrates a side view of the catheter 1200 showing the relative positioning of nozzle 1218 with respect to electrodes 1202-1208 on catheter body 1201.

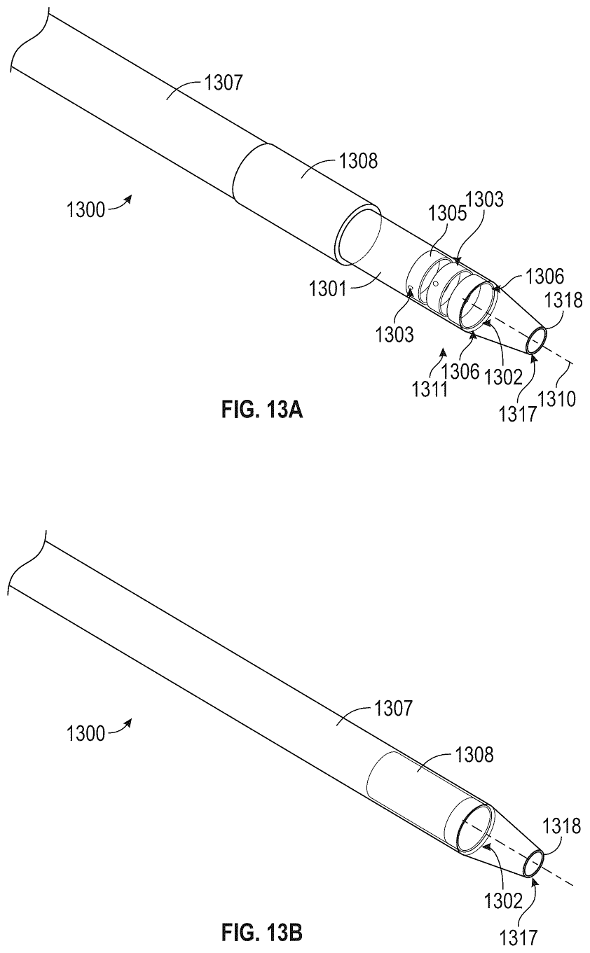

FIGS. 13A and 13B illustrate aspects of a distal portion of a shock wave catheter 1300 that can be used for catheter 10 of FIG. 1 and include yet another exemplary configuration of shock wave emitters and a nozzle, according to one or more aspects of the present disclosure. The catheter 1300 includes a catheter body 1301. The catheter body 1301 includes a cavity 1302 at its distal end that opens in a distal direction. The catheter 1300 includes one or more radially firing shock wave emitters 1303 located outwardly of and adjacent to the cavity 1302. The one or more radially firing shock wave emitters 1303 can generate shock waves that propagate radially outwardly relative to the longitudinal axis 1310 of the catheter body 1301.

The catheter body 1301 includes one or more forward firing shock wave emitters 206 configured to generate shock waves that propagate in a forward direction, distally of the distal end 1311 of the catheter body 201 and which are directed within a nozzle 1318 toward a nozzle outlet 1317. The shock waves may be concentrated at the nozzle outlet 1317 and propagate distally of the nozzle outlet 1317 as described throughout.

One or more of the radially firing shock wave emitters 1303 and/or one or more of the forward firing shock wave emitters 1306 may be formed, in part, by one or more emitter bands 1305 that extend at least partially around the longitudinal axis 1310. The illustrated example includes three emitter bands 1305, the two proximal emitter bands 1305 forming radially firing shock wave emitters 1303 and the distal emitter band forming forward firing shock wave emitters 1306. In some examples, one or more radially firing shock wave emitter 1303 and/or one or more of the forward firing shock wave emitters 1306 may be formed by optical fibers extending from a power source configured to generate laser pulses. The nozzle 1318 may be positioned distally of the distal emitter band forming forward firing shock wave emitters 1306 such that shock wave generated by the forward firing shock wave emitters 1306 are directed into the nozzle 1318 and toward outlet 1317 to concentrate at the outlet and propagate distally of the outlet 1317 and catheter body 1301.

A sheath 1307 may extend around the catheter body 1301 and may include a shield 1308 at its distal end. The sheath 1307 may be movable relative to the catheter body 1301 in the longitudinal direction of the catheter body 1301. FIG. 13A shows the sheath 1307 in a retracted position relative to the catheter body 1301, and FIG. 13B shows the sheath in an extended position relative to the catheter body 1301. When in the extended position of FIG. 13B, the shield 1308 may cover one or more of the radially firing shock wave emitters 1303. In the illustrated example, the shield 1308 is configured to cover all of the radially firing shock wave emitters 1303. The shield 1308 is made of a material that can reflect the shock waves generated by the radially firing shock wave emitters 1303. As such, when the shield 1308 covers one or more of the radially firing shock wave emitters 1303, shock waves generated by the covered radially firing shock wave emitters 1303 are reflected inwardly into the cavity 1302. The reflected shock waves can break up target material (e.g., calcified, multi-morphology, and/or fibrotic material) disposed at least partially within the cavity 1302. The shock waves may propagate in a forward direction and impact target material located in front of the distal end 1311 of the catheter body 1301.

With the sheath 1307 in a retracted position illustrated in FIG. 13A, the radially firing shock wave emitters 1303 are uncovered. Shock waves generated by the one or more radially firing shock wave emitters 1303 when uncovered may propagate radially outwardly to impact target material located radially outwardly of the catheter 1300. Thus, the radially firing shock wave emitters 1303 may be used selectively to break up target material located at least partially within the cavity 1302 and/or target material located radially outward of the catheter 1300 simply by extending or retracting the sheath 1307. As noted above, the catheter 1300 may include one or more forward firing shock wave emitters 1306 that can be used to direct shock waves in a forward direction to break up target material located forward of the catheter, providing yet another mode of action of the catheter 1300. The forward directed shock waves may be concentrated by nozzle 1318 at the nozzles outlet 1317, amplifying the forces exerted by the shock waves on the target material distally of the outlet 1317. These three modes for generating shock waves-generating radially-outwardly directed shock waves, generating radially-inwardly directed shock waves, and generating forward directed shock waves-can be used independently and/or in concert to treat target material (e.g., to break up calcifications, multi-morphic, and/or fibrotic tissue). For example, with reference to the illustration of FIG. 1, catheter 1300 can be used to tunnel into the stenotic lesion by generating shock waves from the forward firing shock wave emitter(s) 1306 and/or from the radially firing shock wave emitters 1303 while the shield 1308 is in an extended position to break up portions of the stenotic lesion positioned within the cavity 1302 and/or in front of the catheter. The shield 1308 can then be retracted and shock waves can be generated by the radially firing shock wave emitters 1303 to break up portions of the stenotic lesion located radially outwardly of the catheter.

The sheath 1307 may be formed from at least one reinforced wire material. The wire material can braided, coiled, or both. The wire material may be round or may be flat to provide a lower profile. The sheath 1307 may be configured to contribute mechanical strength to the catheter 1300. For instance, the material composition of the sheath 1307 could provide increased torqueability, pushability, and/or enhanced rigidity to the catheter 1300 to facilitate maneuvering the catheter 1300 through a patient's vasculature. The sheath 1307 can be laminated with one or more polymer liners. A polymer liner can be formed of any suitable material (e.g., nylon) to allow for improved mechanical properties such as pushability and torqueability.

The shield 1308 may be formed from a hard material that is capable of reflecting shock waves (e.g., stainless steel, platinum-iridium alloy, chromium, etc.). The shield 1308 can be formed of a radiopaque material or include radiopaque material to facilitate fluoroscopic tracking of the catheter 1300. The shield 1308 may be mounted to the sheath 1307 in any suitable fashion, such as via a press fit between the shield 1308 and the sheath 1307 and/or an adhesive attachment between the shield 1308 and the sheath 1307. In some embodiments, the sheath 1307 is made of a material that can reflect shock waves such that the shield 1308 is not a separate component but, rather, a distal region of the sheath 1307.

The catheter body 1301 (and any other exemplary catheters described herein) may be made of any suitable material. Examples of suitable material include urethane, polyether block amide (e.g., Pebax), and other low durometer polymer material.

FIGS. 14A-14C illustrate cross sectional views of an example of the distal portion of catheter 1300 of FIGS. 13A and 13B. FIG. 14A shows the catheter 1300 with the sheath 1307 in a retracted position and FIGS. 14B and 14C show the catheter 1300 with the sheath 1307 in different extended positions. The catheter body 1301 may include an outer wall 1401 and an inner wall 1402 that are spaced from one another to form an annular lumen 1410. The annular lumen 1410 may house at least one radially firing shock wave emitter located radially inwardly of the outer wall 1401 and radially outwardly of the inner wall 1402 and may be filled with a conductive fluid. The illustrated example includes four radially firing shock wave emitters—1303a, 1303b, 1303c, and 1303c. The proximal radially firing shock wave emitters 1303a and 1303b in the illustrated example are located at the same longitudinal position but at opposite circumferential positions. The distal radially firing shock wave emitters 1303c and 1303d in the illustrated example are located at the same longitudinal position, distal of the proximal radially firing shock wave emitters 1303a and 1303b, and are located at opposite circumferential positions. The illustrated arrangement of the radially firing shock wave emitters is merely exemplary. The catheter can include any number of radially firing shock wave emitters in any longitudinal and circumferential locations.

The inner wall 1402 may form a cavity 1302 that has an open distal end 1420. The inner wall 1402 may reduce in diameter from a larger diameter section 1422 that defines the cavity 1302 to a smaller diameter section 1424 that defines a central lumen 1411. The larger diameter section 1422 may have a diameter of up to 1 millimeters (mm), up to 0.750 millimeters (mm), up to 0.500 millimeters (mm), or up to 0.250 millimeters (mm). The diameter of the larger diameter section 1422 may be at least 0.010 millimeters (mm), at least 0.020 millimeters (mm), or at least 0.050 millimeters (mm). In some examples, the diameter of the larger diameter section 1422 is in the range of 0.050 millimeters (mm) to 0.250 millimeters (mm). A length of the cavity 1302 may be up to 3 cm, up to 2 cm, or up to 1 cm. The length of the cavity 1302 may be at least 1 millimeters (mm), at least 2 mm, or at least 3 millimeters (mm). In some examples, the length of the cavity 1302 is in the range of 3 millimeters (mm) to 1 cm. The central lumen 1411 may receive a guidewire and/or a pacemaker lead. The inner wall 1402 may include a narrowing section 1426 that transitions from the larger diameter section 1422 to the smaller diameter section 1424. The narrowing section 1426 can have any suitable shape, including a tapering shape as shown, a stepped shape, a domed shape, or a funnel shape.

When the sheath 1307 is in a retracted position such that the shield 1308 is not covering the radially firing shock wave emitters 1303a-d, as illustrated in FIG. 14A, shock waves generated by the radially firing shock wave emitters 1303a-d propagate radially outwardly relative to the longitudinal axis 1310 in direction 1412. These shock waves can break up calcifications, multi-morphology tissue, or fibrotic tissue (not shown) located radially outwardly of the catheter 1300.

When the sheath 1307 is in an extended position such that shield 1308 is covering one or more of the radially firing shock wave emitters 1303a-d, as illustrated in FIGS. 14B and 14C, shock waves generated by the covered radially firing shock wave emitters 1303a-d propagate radially outwardly in direction 1412 and are then reflected by shield 1308 radially inwardly (e.g., in direction 1414) into the cavity 1302. The reflected shock waves may impinge on calcified or fibrotic material located within the cavity 1302 and/or be directed distally toward an outlet 1317 of nozzle 1318 toward a target treatment area located distally of the nozzle outlet 1317. Shock waves may constructively interfere within the cavity 1302, amplifying the destructive effect of the shock waves on the calcified or fibrotic material. Shock waves may be reflected multiple times by the shield 1308. Shock waves may propagate in a distal direction, out through the open distal end 1420 of the cavity 1302 into the nozzle 1318 and through the nozzle outlet 1317. Optionally, the narrowing section 1426 is configured to reflect shock waves in a distal direction. As such, the catheter 1300 can be used with the shield 1308 covering the radially firing shock emitters 1303a-d for treating target material located within the cavity 1302 and/or in front of the cavity 1302.

While FIG. 14B shows the shield 1308 covering all of the radially firing shock wave emitters 1303a-d, the shield 1308 may be positioned so that it covers one or more of the shock wave emitters 1303a-d but not all of them, enabling some shock waves to propagate radially outwardly and others to be reflected radially inwardly. An example of this is illustrated in FIG. 14C in which the shield is positioned to cover radially firing shock wave emitters 1303a and 1303b but not radially firing shock wave emitters 1303c and 1303d. By covering some but not all of the radially firing emitters, the catheter 1300 could be used to treat scarred, calcified, multi-morphic, and/or fibrotic material or other target material located both outside of the catheter 1300 and within and/or in front of the cavity 1302.

Optionally, at least one forward firing shock wave emitter 1306 may be positioned in the annular lumen 1410, at the distal end 1311 of the catheter body 1301, distally of the radially firing shock wave emitters 1303a-d. The one or more forward firing shock wave emitters 1306 can be configured to generate shock waves directed forward in direction 1413, past the distal end of the catheter 1300 via nozzle 1318 and through the nozzle outlet 1317, such as to break up calcifications, multi-morphology tissue, or fibrotic tissue (not shown) located forward of the catheter 1300. The one or more forward firing shock wave emitters 1306 could be used to treat calcified, multi-morphic, and/or fibrotic material located in front of the catheter 1300 and the radially firing shock wave emitters 1303 could be used to treat calcified, multi-morphic, and/or fibrotic material located in radially outward of the catheter 1300 and/or within the cavity 1302.

The one or more radially firing shock wave emitters 1303a-d and the one or more forward firing shock wave emitters 1306 may generate shock waves based on voltage pulses applied to the emitters from a voltage pulse generator (e.g., shock wave power source 28 of FIG. 1). A plurality of conductors 1403 may extend in the annular lumen 1410 to the radially firing shock wave emitters 1303 and/or forward firing shock wave emitters 1306 to provide voltage pulses to the shock wave emitters for generating shock waves. The plurality of conductors 1403 may electrically connect the one or more radially firing shock wave emitters 1303 and/or the one or more forward firing shock wave emitters 1306 to a shock wave power source and/or to each other.

In the illustrated example, the conductors 1403 are configured so that the radially firing shock wave emitters 1303a-d are arranged serially such that a voltage pulse can cause each of the radially firing shock wave emitters 1303a-d to generate a shock wave. Each of the radially firing shock wave emitters 1303a-d is formed by an electrode pair that includes an end of one of the conductors 1403 and a portion of an emitter band 1305.

A first conductor 1404 extends proximally to a first emitter band 1305a. The first emitter band 1305a extends around the longitudinal axis 1310, within the annular lumen 1410. The distal end of the first conductor 1404 is uninsulated and is located adjacent to but spaced from a first hole 1416a in the first emitter band 1305a. The distal end of the first conductor 1404 and the first emitter band 1305a together form an electrode pair of radially firing shock wave emitter 1303a. In use, a suitable voltage pulse applied to the electrode pair formed by the distal end of the first conductor 1404 and the first emitter band 1305a causes an electrical arc to form across the gap between them in conductive fluid that fills the annular lumen 1410, which results in the generation of one or more shock waves.

Radially firing shock wave emitter 1303b is formed by the first emitter band 1305a and a proximal end of a second conductor 1405. The proximal end of the second conductor 1405 is uninsulated and located adjacent to but spaced from a second hole 1416b in the first emitter band 1305a. The proximal end of the second conductor 1405 and the first emitter band 1305a form the electrode pair of radially firing shock wave emitter 1303a.

The second conductor 1405 extends distally to a second emitter band 1305b, which extends around the longitudinal axis 1310 within the annular lumen 1410. The distal end of the second conductor 1405 is uninsulated and adjacent to but spaced from a hole 1416c in the second emitter band 1305b. The distal end of the second conductor 1405 and the second emitter band 1305b together form the electrode pair of radially firing shock wave emitter 1303c. Radially firing shock wave emitter 1303d is formed by the second emitter band 1305b and an uninsulated distal end of a third conductor 1419, which is adjacent to but spaced from a hole 1416d in the second emitter band 1305b. The third conductor 1419 extends toward a proximal end of the catheter 1300 for connection to a voltage source (directly or via one or more intermediate conductors).

In use, a voltage may be applied across first conductor 1404 and third conductor 1419 (e.g., across proximal ends of the first conductor 1404 and third conductor 1419) that causes current to flow across the gap between the distal end of the first conductor 1404 and the first emitter band 1305a, through the first emitter band 1305a, across the gap between the first emitter band 1305a and the proximal end of the second conductor 1405, along the second conductor 305, across the gap between the distal end of the second conductor 1405 and the second emitter band 1305b, through the second emitter band 1305b, and across the gap between the second emitter band 1305b and the distal end of the third conductor 1419 resulting in shock waves being generated at each of the radially firing shock wave emitters 1303a-d.

Additional conductors may be provided for providing voltage pulses to one or more forward firing shock wave emitters 1306. The illustrated example includes two conductors 1428 and 1430 that provide voltage pulses to two forward firing shock wave emitters 1306a and 1306b. The forward firing shock wave emitters 1306a and 1306b include electrode pairs formed by a distal end of a respective one of the conductors 1428 and 1430 and a third emitter band 1305c. Distal ends of each of the conductors 1428 and 1430 are spaced by respective gaps from the distal end of the third emitter band 1305c. Voltage pulses can be applied to the conductors 1428 and 1430 so that sparks form across the gap between conductor 1428 and the third emitter band 1305c and across the gap between conductor 1430 and the third emitter band 1305c (which, in the illustrated example, is opposite the gap between conductor 1428 and the third emitter band 1305c), generating shock waves at those two locations that propagate in a distal direction as indicated by arrows 1413.

The arrangement of the radially firing shock wave emitters 1303a-d and forward firing shock wave emitters 1306a-b illustrated in FIGS. 14A and 14B is merely exemplary. Any number, arrangement, and configuration of radially firing and/or forward firing emitters may be used. For example, one or more radially firing shock wave emitters may be formed by gaps between two wires, rather than between a wire and an emitter band. In some examples, radially firing shock wave emitters are formed by locating conductors adjacent to an end of an emitter band or a notch in the end of an emitter band, rather than a hole in the emitter band. In some examples, radially firing shock wave emitters are circumferentially spaced 180 degrees from each other (such as shown in FIG. 14A). However, any circumferential spacing may be used, including 60 degrees, 90 degrees, 120 degrees, etc. In some examples, emitters provided by each emitter band are circumferentially spaced from each other by 180 degrees and adjacent emitter bands are arranged such that a set of emitters formed by a first band are circumferentially offset from a set of emitters formed by a second emitter band, such as by 90 degrees, so that, collectively, emitters are spaced at 90 degree intervals.

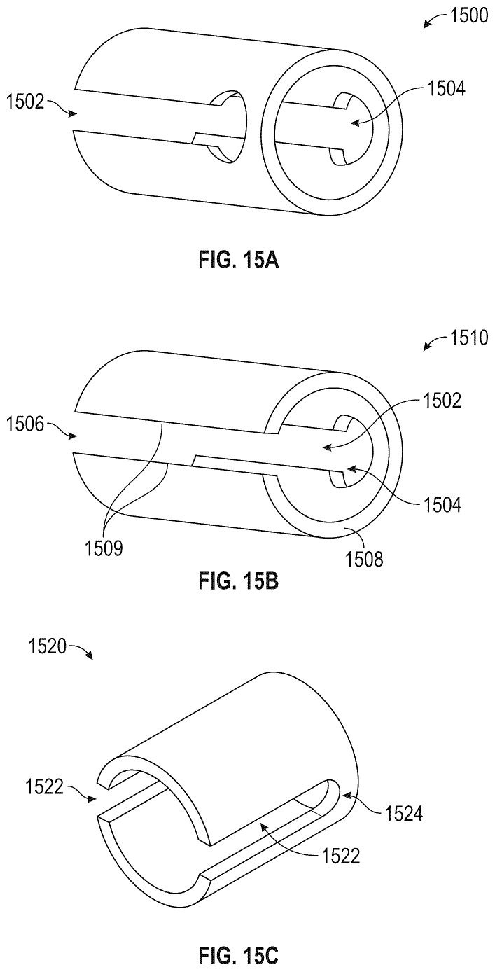

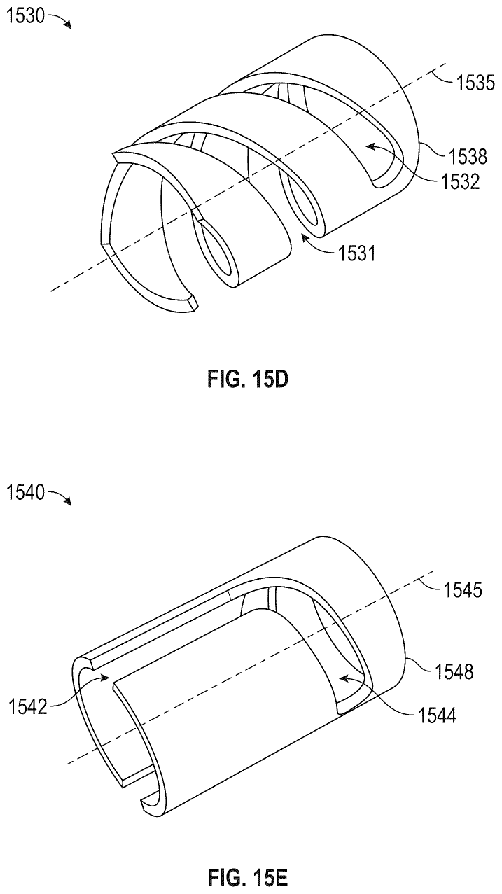

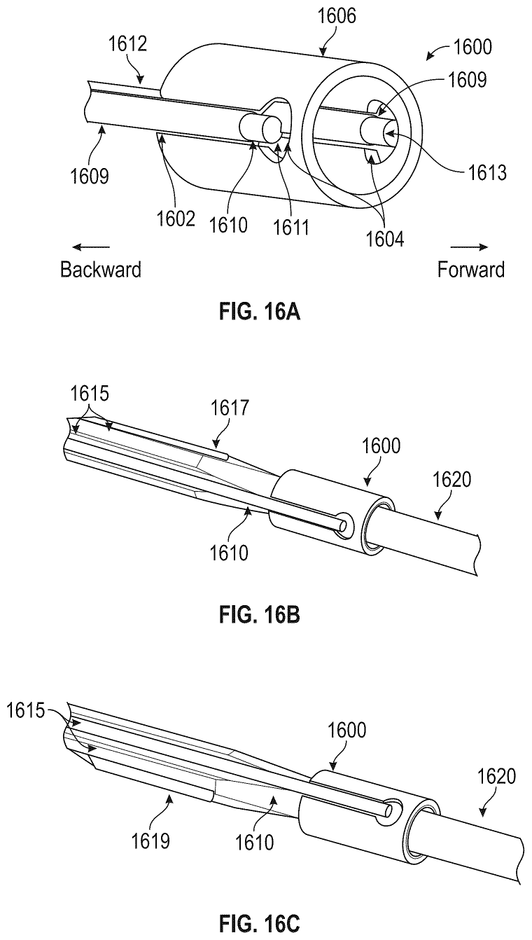

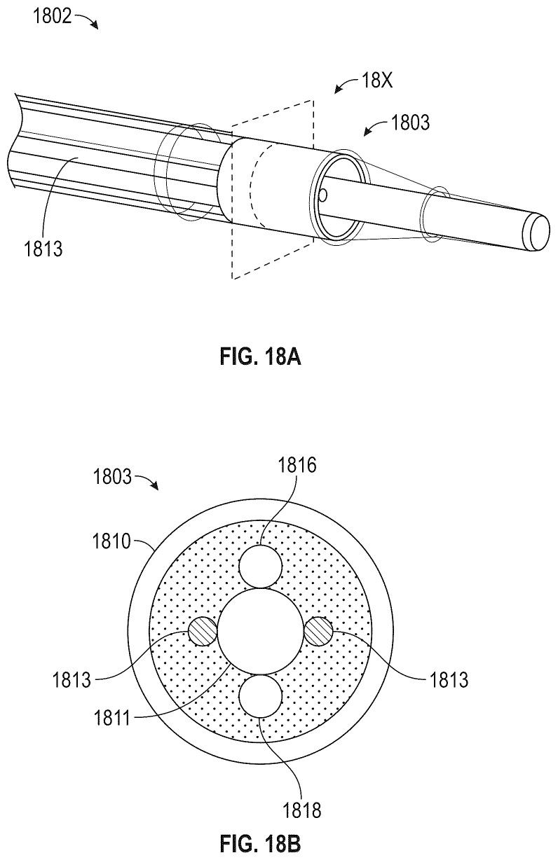

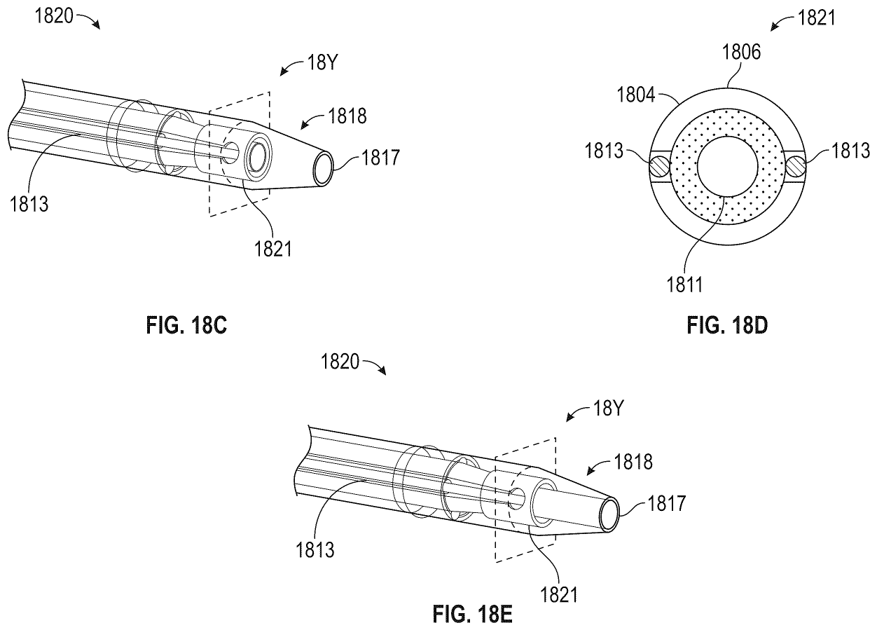

FIGS. 15A-15E below illustrate exemplary slotted emitter bands that form an electrode of forward biased shock wave emitters as illustrated in FIGS. 16A-16C. The slotted emitter bands can be positioned on a catheter that includes a nozzle configured to concentrate at least a portion of the forward biased shock waves at an outlet of the nozzle as described throughout and as shown in FIGS. 18C-18E.

FIG. 15A illustrates a perspective view of an exemplary slotted emitter sheath 1500 of an emitter having slots with a circular end. The emitter sheath 1500 can be incorporated into a shock wave generator such as the shock wave generator 16 of catheter 10 of FIG. 1 and may serve as an electrode of an electrode pair in an emitter. The emitter sheath 1500 includes a pair of slots 1502 extending along a length of the emitter sheath 1500 that terminate with a circular cutout 1504. The cut-out can be any variety of geometries, such as a triangular, square, rectangular, octagonal, hexagonal, elliptical, etc., and the circular shape used herein is provided for example only. Incorporating a shape with smoothed edges (e.g., circular, elliptical, etc.) rather than a shape with sharp edges may improve the structural integrity of the emitter sheath 1500, because shapes with sharp edges may introduce residual stress peaks that tend to crack the sheath of the emitter sheath 1500. The slots 1502 and cutout 1504 regions can enable placement of a wire or another electrode proximate to the distal end of the emitter sheath 1500, which can encourage forward-biased shock waves.

FIG. 15B illustrates another variation of a slotted emitter sheath 1510 that can be incorporated in an emitter of a catheter. The emitter sheath 1510 can be incorporated into a shock wave generator such as the shock wave generator 16 of catheter 10 of FIG. 1 and may serve as an electrode of an electrode pair in an emitter. The slotted emitter sheath 1510 includes a first slot 1502 that terminates with a circular cutout 1504 and a second slot 1506 that extends across the entire length of the emitter sheath 1510. Again, the circular cutout 1504 is exemplary and may be implemented with any number of shapes. The second slot 1506 can enable placement of a wire or another electrode near the distal face 1508 of the emitter sheath 1510, which can encourage forward-biased shock waves.

FIG. 15C illustrates another variation of a slotted emitter sheath 1520 that can be incorporated in an emitter of a catheter. The emitter sheath 1520 can be incorporated into a shock wave generator such as the shock wave generator 16 of catheter 10 of FIG. 1 and may serve as an electrode of an electrode pair in an emitter. The slotted emitter sheath 1520 includes two slots 1522 that terminate with a rounded cutout 1524. Incorporating rounded cutouts 1524 may promote forward-biased shock waves, in that the shock waves generated when a wire or another electrode is placed near the distal end of the slot 1522 close to the distal end of the rounded cutout 1524.

In variations in which the slotted emitter sheath serves as an electrode of an electrode pair in an emitter, the other electrode of the pair can include a wire that is positioned within the slot, as will be discussed further below. When voltage is supplied to a wire and across an electrode pair and shock waves are generated, however, the most distal portion of the wire can erode (e.g., retreat from the distal end of the wire towards the proximal end of the wire). As the wire erodes and the furthest distal portion recedes, the origin point from which shock waves are generated may also recede. Accordingly, it may be beneficial for the slot of an emitter to include at least a portion that extends circumferentially around the emitter sheath, rather than only along a longitudinal axis of the emitter sheath.

FIG. 15D illustrates another variation of a slotted emitter sheath 1530 that can be incorporated in an emitter of a catheter. The emitter sheath 1530 can be incorporated into a shock wave generator such as the shock wave generator 16 of catheter 10 of FIG. 1 and may serve as an electrode of an electrode pair in an emitter. The slotted emitter sheath 1530 includes two helical slots 1531 and 1532, with at least a portion of these slots extending circumferentially around the emitter sheath relative to the longitudinal axis 1535 of the emitter sheath. Wires that lay within the path of either helical slots 1531 and 1532 can be insulated albeit with exposed distal ends, which act as electrode surfaces forming electrode pairs with the material of emitter sheath 1530. The helical shape of the helical slots 1531 and 1532 can ensure that even as a wire retreats in a proximal direction due to erosion from shock wave generation, the wire is following the path of helical slot 1531 and/or 1532, and the origin point of the shock waves (jumping from the distal end of the wire to the slotted emitter sheath 1530) remains adjacent to the distal end 1538 of the slotted emitter sheath 1530 for a longer period. Accordingly, incorporating a helical slotted sheath can promote forward biased shock waves for a longer period than an emitter sheath having one or more slots that do not have a helical path.

Another design which similarly encourages forward biased shock waves for a longer period is shown in FIG. 15E, which illustrates another variation of a slotted emitter sheath 1540 that can be incorporated in an emitter of a catheter. The emitter sheath 1540 can be incorporated into a shock wave generator such as the shock wave generator 16 of catheter 10 of FIG. 1 and may serve as an electrode of an electrode pair in an emitter. The slotted emitter sheath 1540 includes a pair of slots 1542 that terminate with a contorted end 1544 (colloquially referred to as a “hockey stick” path), which extends circumferentially around the emitter sheath relative to the longitudinal axis 1545 of the emitter sheath. Wires that lay within the path of either slot 1542 with contorted end 1544 can be insulated albeit with exposed distal ends, which act as electrode surfaces forming electrode pairs with the material of emitter sheath 1540. Similarly to the helical slots above, the contorted end 1544 can promote forward biased shock waves for a longer period. For example, if a wire is placed within the slot 1542 and then shock waves are generated, even as the wire retreats within the slot due to erosion from shock wave generation (e.g., retreating from the distal end of the wire towards the proximal end of the wire), the distal end of the wire nonetheless remains roughly parallel to the distal end 1548 of the slotted emitter sheath 1540 for a longer period than an emitter sheath having slots that do not include a contorted end.

The emitter sheath 1500 is a generally cylindrical sheath. The emitter sheath 1500 may be formed from a variety of lightweight conductive materials, including metals and alloys such as stainless steel, cobalt chromium, platinum chromium, cobalt chromium platinum palladium iridium, or platinum iridium, or a mixture of such materials. In one or more examples, a catheter may include a plurality of slotted emitters positioned at various locations along a length of the catheter (e.g., longitudinally spaced apart from one another), and may include a combination of slotted emitters with any variation of slots, such as the slots with circular cutouts as shown with respect to the slotted emitter sheath 1500, through cut slots as shown with respect to the slotted emitter sheath 1510, slots with rounded ends as shown with respect to the slotted emitter sheath 1520, helical slots as shown with respect to the slotted emitter sheath 1530, and/or contorted slots as shown with respect to the slotted emitter sheath 1540.

FIG. 16A illustrates an exemplary emitter 1600, according to aspects of the present disclosure. The emitter 1600 includes an emitter sheath 1606, a lead wire 1610, and a return wire 1612. The emitter sheath 1606 here contains a pair of slots 1602 that each terminate with a circular cutout 1604, like the emitter sheath 1500 of FIG. 15A. The lead wire 1610 is positioned in one of the slots 1602 such that a distal face 1611 of the lead wire 1610 is located in the circular cutout 1604. Similarly, the return wire 1612 is positioned in the other slot 1602 such that the distal face 1613 of the return wire 1612 is located in the circular cutout 1604.

Each of the lead wire 1610 and the return wire 1612 can be insulated wires with insulation 1609 extending along the length of the wire (e.g., from a proximal connection to a voltage source to a distal position as part of an electrode assembly). The wires may be cylindrical wires (as shown in FIG. 16A) or may be flat wires. Optionally, the wires may include a flattened or crimped portion, such as a crimped distal end. The wires may have a diameter that is the same as the thickness of the emitter sheath 1606 such that the wires are coplanar with the outer surface of the emitter sheath 1606. The wires may instead have a greater diameter than the emitter sheath 1606 such that the wires extend farther outward than the emitter sheath 1606. When the wires have a diameter that is greater than the thickness of the emitter sheath 1606, the cavitation bubbles and/or shock waves generated by the emitter 1600 may propagate generally outward in all directions from the distal end of each wire.

At least a portion of the lead wire 1610 is exposed to form an electrode of an electrode pair opposite a section of electrode sheath 1606 of the emitter 1600. Similarly, at least a portion of the return wire 1612 is exposed to form an electrode of an electrode pair opposite a section of electrode sheath 1606 of the emitter 1600. The exposed portion (e.g., the non-insulated or insulated removed portion) of each wire can be an area of the wire wherein the insulating layer that surrounds the insulated wire is exposed, or wherein a strip of the insulating layer is removed. The insulation-removed portion may include just the distal faces 1611 and 1613 of the lead wire 1610 and return wire 1612. Optionally, the non-insulated portion of the wires can include a larger portion of the wire than just the distal face or distal end. For instance, the distal tip, including a portion of the shaft of the wire and the distal face, may form the non-insulated portion of the wire (as depicted in FIG. 16A). As shown in FIG. 16A, both an insulated portion and a non-insulated exposed portion of the lead wire 1610 and return wire 1612 is positioned in the slot 1602. Where the non-insulated portion of the wires is the distal end of the wire, a majority of the length of the slot includes the insulated portion of the wire. Optionally, rather than including a distal end that is located in the slot, the return wire can be connected to a proximal end of the conductive sheath.

The emitter 1600 includes two electrode pairs, a first pair including the distal face 1611 of the lead wire 1610 and a first circular cutout 1604 of the emitter sheath 1606 (more particularly, a surface of the circular cutout that is proximate to the distal face 1611), and a second pair including the distal face 1613 of the return wire 1612 and a second circular cutout 1604 of the emitter sheath 1606. Where the emitter instead includes a slot that extends along the entire length of the emitter (e.g., slot 1506 of FIG. 15B), a surface of the slot and/or of the distal end of the emitter sheath forms the electrode of the electrode pair. For example, referring to FIG. 15B, the portion of the emitter sheath 1510 that forms an electrode can be the distal face 308 and/or one or both inner faces 1509 of the slot 1506.

The distal faces 1611 and 1613 of the lead wire 1610 and return wire 1612 are each separated from an inner surface of the circular cutout 1604 of the emitter sheath 1606 by a gap. When voltage is applied across the lead wire 1610 and the return wire 1612, current flows across the gaps to generate shock waves. For instance, current may flow from the distal face 1611 of the lead wire 1610 to the emitter sheath 1606 by jumping across the gap between the distal face 1611 and the inner face of the cutout 1604 and then travel from the emitter sheath 1606 to the return wire 1612 by jumping across the gap between the inner face of the cutout 1604 to the distal face 1613 of the return wire 1612.

The lead wire 1610 receives voltage from a voltage source (such as voltage source 28 of FIG. 1) and delivers that voltage to the emitter sheath 1606. The return wire 1612 receives voltage from the emitter sheath 1606 and returns that voltage to the voltage source (e.g., to complete the circuit). In one or more examples, the lead wire 1610 (also called the “hot wire”) may deliver greater voltage. Thus, the gap adjacent to the lead wire 1610 can generate a larger shock wave that exhibits increased pressure than the shock wave generated adjacent to the return wire 1612. The lead wire 1610 may be located in a cutout 1604 as shown in FIG. 16A, and/or may be located in a slot that extends along the entire length of the emitter sheath 1606. Similarly, the return wire 1612 may be located in a cutout such as cutout 1604 and/or a slot that extends along the entire length of the emitter sheath 1606. In one or more examples, the polarity of the lead wire 1610 and the return wire 1612 may be switched. In such case, the return wire 1612 will act as the “lead” and deliver voltage to the emitter sheath 1606 while the lead wire 1610 will act as the “return” and return that voltage to the voltage source. Accordingly, the terminology of “lead” and “return” as it relates to the lead wire 1610 and the return wire 1612 is provided for example only, as both wires may serve as a “lead” or “return” depending on the polarity of the wires as they connect to a voltage source. Additionally, polarity may be switched during use such that a lead wire in one instance is the return wire in another instance.

By locating the lead wire 1610 and return wire 1612 in the slots 1602 such that the insulation-removed portions (e.g., the distal faces 1611 and 1613) are located proximate to a distal end of the emitter sheath 1606, the emitter 1600 promotes forward-biased and/or distally directed shock waves that are generated when current jumps across the gaps between the electrodes of each respective electrode pair. That is, shock waves generated when current jumps, for example, from the distal face 1611 of the lead wire 1610 to the emitter sheath 1606, will propagate in a forward direction (e.g., to the right based on the orientation shown in FIG. 16A). Encouraging shock waves to propagate in a forward direction, i.e., in a forward-biased configuration, can more efficiently break up occlusions that are proximate to a forward end of the catheter, and thus can be desirable when treating a tight occlusion, such as a CTO, because it enables the catheter to be incrementally advanced farther within the occlusion and/or to create a channel through the occlusion. As described throughout and as illustrated with reference to the slotted emitter band(s) in FIGS. 18C-18E, the forward biased shock waved can be directed into a nozzle and directed to a nozzle outlet, thus concentrating the shock waves together to create a more concentrated force in the direction of the nozzle outlet.

In addition to promoting forward-biased shock waves, locating the lead wire 1610 and return wire 1612 in the slots 1602 of the emitter sheath 1606 also reduces the overall diameter of the emitter 1600 relative to a configuration wherein the wires are located within the emitter sheath 1606 (e.g., in the interior of the emitter sheath 1606). Reducing the overall diameter of the emitter 1600 improves the navigability of the catheter within tight occlusions, as it enables the catheter to be advanced within smaller spaces than a catheter with a larger overall diameter.

Another design configuration that reduces the overall diameter of the catheter is incorporating grooves in the elongate tube that receive the wires of the emitter 1600. FIG. 16B illustrates a top perspective view of the emitter 1600 mounted to an elongate tube 1620, as the emitter 1600 may be mounted within a catheter. The elongate tube 1620 includes one or more grooves 1615 that extend along the length of the elongate tube. The lead wire 1610 is positioned in one of the grooves 1615. On the opposite side, the return wire can also be positioned in another groove (not visible in figure). By locating the wires (e.g., lead wire 1610 and return wire 1612) in grooves rather than on the outer surface of the elongate tube 1620, the overall diameter of the emitter 1600 when mounted to the elongate tube 1620 is reduced. Accordingly, a catheter that incorporates the emitter 1600 with the elongate tube 1620 having grooves 1615 exhibits improved navigability and crossing ability relative to a catheter with an elongate tube without grooves.

As shown in FIG. 16B, an inlet lumen 1617 is positioned in one of the grooves 1615 of the elongate tube 1620. An outlet lumen 1619 is shown in FIG. 16C, which illustrates the bottom perspective view of the emitter 1600 mounted to the elongate tube 1620. The inlet lumen 1617 and outlet lumen 1619 can be used to provide a fluid to an internal region of a nozzle (e.g., nozzle 1818 illustrated in FIG. 18C) to replace fluid pushed out from the nozzle outlet during shock wave generation and/or to aspirate debris from the nozzle following shock wave generation.

The placement and spacing of the electrode pairs can be controlled to provide a more effective shock wave treatment. For instance, the electrode pairs of a shock wave generator may be spaced circumferentially around the distal end of the catheter in consistent increments, e.g., 180 degrees apart or 90 degrees apart, to generate shock waves evenly around the catheter. The electrode pairs of the emitter 1600 of FIG. 16A are circumferentially spaced apart from one another by 180 degrees (e.g., on opposite sides of the emitter sheath 1606 from one another). In one or more examples, the electrode pairs may instead by circumferentially spaced apart from one another (which can be referred to as “offset”) by less than 180 degrees, which may encourage the shock waves generated by the respective electrode pairs to constructively interfere with one another. Moreover, the emitter 1600 may include more than two electrode pairs to encourage the generation of additional shock waves when voltage is supplied to the emitter sheath 1606.

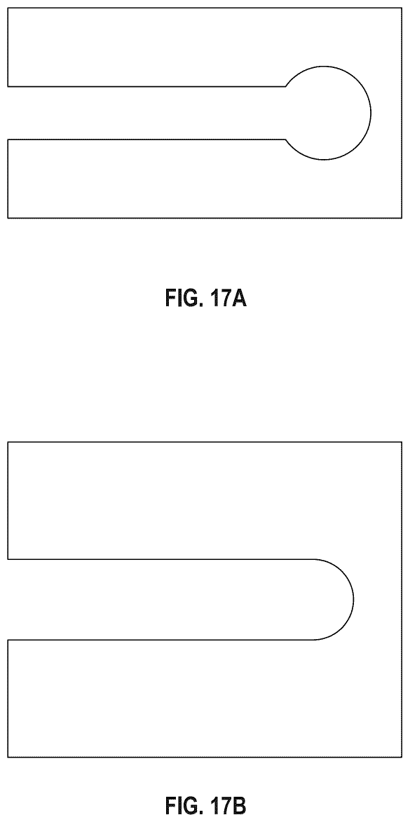

FIG. 17A illustrates a plan drawing showing a slotted emitter sheath. According to one or more examples, the dimension “A,” which corresponds to the width of a slot (e.g., slot 1602 of FIG. 16A), may be 0.008 inches. Dimension “B,” which corresponds to the diameter of a circular cutout (e.g., cutout 1604 of FIG. 16A), may be 0.014 inches. Dimension “C,” which corresponds to a distance from the distal end of the emitter sheath to the most distal portion of the cutout, may be 0.005 inches. In one or more examples, dimension “C” may instead be 0.010 inches. Dimension “D,” which corresponds to a distance from the distal end of the emitter sheath to a center point of the cutout may be 0.012 inches. Optionally, dimension “D” may be 0.017 inches. In one or more examples, dimension “D” may correspond to the most distal end of a wire when the wire is positioned in the slot. Dimension “E,” which corresponds to the length of the slotted emitter sheath may be, for example between about 0.02-0.12 inches. In one or more examples, the length of the slotted emitter sheath may be more than 0.12 inches, such as 0.2 inches 0.3 inches, 0.4 inches, and increments and gradients of length therein. In one or more examples, the length of the slotted emitter sheath may be less than 0.02 inches, such as 0.01 inches.

FIG. 17B illustrates a plan drawing showing a slotted emitter sheath. As compared to FIG. 17A, the slot of slotted emitter sheath depicted in FIG. 17B does not terminate with a circular cutout. Instead, the slot terminates with a curved distal end. In one or more examples, rather than a curved distal end, the slot may terminate with a straight distal end (e.g., a rectangular cutout). The dimensions of the slotted emitter sheath of FIG. 17B, namely the width of the slot (dimension “A”), the distance from the distal end of the emitter sheath to the most distal portion of the cutout (dimension “C”), and the distance from the distal end of the emitter sheath to a center point of the distal portion of the slot (dimension “D”) may be the same as the measurements of the slot of FIG. 17A.

FIG. 18A illustrates the distal end of an exemplary catheter 1802 with a shock wave generator including an emitter 1803 that surrounds a number of lumens, and FIG. 18B illustrates the cross-sectional view of the catheter 1802 cut across plane 18X. The catheter 1802 has an emitter 1803 having an emitter sheath 1810 that surrounds a pair of insulated wires 1813, a guidewire lumen 1811 that receives a guidewire, an inlet lumen 1816 and an outlet lumen 1818. As visible in FIG. 18B, the emitter sheath 1810 is the outermost component of the emitter 1803 of catheter 1820 and the emitter sheath 1810 surrounds each of the interior lumens. The catheter 1820 has an overall diameter d1. The overall diameter d1 of the catheter 1802 may be, for example, 1.4 millimeters (mm) or 1.5 millimeters (mm), and increments and gradients of range therein.

FIG. 18C illustrates the distal end of an exemplary catheter 1820 with a shock wave generator that has a slotted emitter 1821 and a nozzle 1818 configured to direct forward-biased shock waves generated by slotted emitter 1821 toward an outlet 1817 of nozzle 1818, and FIG. 18D illustrates the cross-sectional view of the catheter 1820 cut across plane 18Y. The catheter 1820 includes a slotted emitter 1821 having an emitter sheath 1806 with a pair of slots 1804 that receive wires 1813. The catheter 1820 can be configured as the catheter 10 of FIG. 1, and can include an electrode assembly such as the emitter 1600 of FIGS. 16A and 16B, and/or an emitter with a slot that extends across the length of the emitter. The catheter 1820 has an overall diameter d2. The overall diameter d2 of the catheter 1820 may be less than 1.5 millimeters (mm). For instance, the overall diameter d2 may be 1.3 millimeters (mm), 1.35 millimeters (mm), 1.40 millimeters (mm), or 1.45 millimeters (mm), and increments and gradients of range therein.

As compared to the catheter 1802, the catheter 1820 includes wires 1813 located in grooves of the elongate tube (such as grooves 415 of elongate tube 420 FIG. 4B) and positioned in the slots 1804 of the emitter sheath 1806. By locating the wires 1813 of the catheter 1820 in slots of an emitter sheath rather than within an emitter sheath and positioning the wires 1813 within grooves along the elongate tube, the overall diameter d2 of the catheter 1820 is less than the diameter d1 of the catheter 1802. Accordingly, the catheter 1820 achieves a smaller crossing profile, which improves the navigability of the catheter 1820, especially within hard-to-cross occlusions. For example, a catheter with a small overall diameter can burrow farther into a tight occlusion or a CTO and to create a channel when crossing the occluded area of the vessel.

Additionally, by positioning the distal end of the wires 1813 proximate to the distal end of the emitter sheath 1806, the origin of the shock waves generated via the emitter 1821 (from the current jumping between the wires and the emitter sheath) is proximate to the distal end of the emitter sheath 1806. Placing the origin of the shock waves proximate to the distal end of the emitter sheath 1806 enables the catheter 1820 to generate shock waves that are forward-biased and with the most distal portion of those shock waves applying spherical pressure against occlusions that are in front of the catheter 1820. Thus, a nozzle 1818 can be positioned at least partially distally of the emitter sheath 1806 and the catheter 1820 and nozzle 1818 can be configured such that the forward biased shock waves are directed to and concentrated at a nozzle outlet 1817 before propagating distally of the nozzle outlet 1817 as described throughout. For instance, a distal portion of the catheter 1820 proximate to emitter sheath 1806 (e.g., the portion enclosing the emitter sheath 1806) and/or the nozzle 1818 can be formed of an acoustically reflective material such that and radially propagating shock waves are reflected internally toward the nozzle 1818 and directed to the nozzle outlet 1817. In some examples, an acoustically reflective surface may be positioned proximally of the emitter sheath 1806 such that any proximally propagating shock waves are reflected toward the nozzle 1818.

In contrast, the origin of the shock waves generated by the catheter 1802 is not as proximate to the distal end of the emitter and no nozzle or acoustically reflective material are provided radially outward of the emitter sheath of catheter 1802. Accordingly, less (or none) of the spherical pressure of the shock waves generated by the catheter 1802 does not impinge against the occlusions that are in front of the catheter 1820 and instead dissipates as it propagates generally outwardly. Accordingly, as compared to the catheter 1802, the catheter 1820 generates forward-biased shock waves and harnesses the distal spherical pressure of these waves using nozzle 1818 to break up occlusions in front of the catheter 1820 thus enabling the catheter 1820 to be advanced farther within tight occlusions.

In one or more examples, a catheter comprising a slotted emitter sheath, such as the catheter 1820, can include of one or more coatings and/or liners that can reduce (or prevent) friction and/or drag when using the catheter. Friction and/or drag may be generated, for example, between the outer surface of a catheter and the vessel and/or between an internal guidewire lumen of the catheter and a guidewire as the catheter is inserted into a body lumen. To reduce friction and/or drag, a catheter can include a coating and/or liner at one or both of these interfaces. For example, the catheter can include a coating and/or liner on a portion or the entirety of an inner surface of a guidewire lumen that receives a guidewire. For instance, the catheter 1820 can include a coating and/or liner on the inner surface of the guidewire lumen 1811 to prevent or reduce friction and/or drag between the guidewire lumen 1811 and a guidewire as the catheter 1820 travels along a guidewire positioned in the guidewire lumen 1811. In addition or alternatively, a catheter can include an external coating and/or a liner on the external surface of the catheter. For example, the catheter 1820 could include a coating and/or liner on the outer surface of the catheter 1820 to prevent or reduce friction and/or drag between the catheter 1820 and the body lumen the catheter 1820 is traveling through.

By incorporating one or more liners and/or coatings that reduce or prevent friction and/or drag, the catheter can travel more easily within the body lumen, which can improve the device tracking and enable the catheter to reach and treat more distal lesions than a catheter without liners and/or coatings. Materials that a liner and/or coating may include that can reduce friction and/or drag include, for example, polymeric materials such as polytetrafluoroethylene (PTFE) and high density polyethylene (HDPE), hydrophilic or hydrophobic coatings, etc.

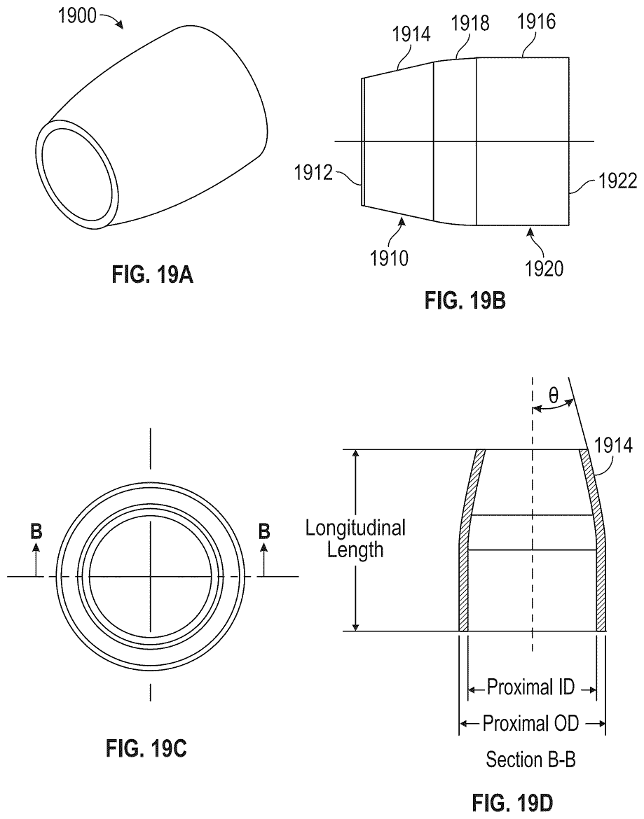

FIGS. 19A-19D illustrate, by way of example, a nozzle 1900 that may form a distal end of the catheters described herein. FIG. 19A illustrates a perspective view; FIG. 19B illustrates a side view; FIG. 19C illustrates a front end view; and FIG. 19D illustrates a cross-sectional view through plane B-B in FIG. 19C. Nozzle 1900 includes a body with a distal region 1910 and a proximal region 1920. Distal region 1910 may have a distal outlet 1912 that is narrower than a proximal outlet 1922 of the proximal region. Distal outlet 1912 may include a distal outlet diameter of 3.0 millimeters (mm) to 15 millimeters (mm). In some examples, the distal outlet diameter is at least 5 millimeters (mm). In some examples, the distal outlet diameter is less than 10 millimeters (mm). In some embodiments, the distal outlet diameter and the proximal outlet diameter are substantially the same. The various geometries of the nozzle may allow for concentrated sonic output delivery to anatomies where positioning shock wave emitters proximate a lesion is difficult or not possible.

The body of nozzle 1900 includes a tapered side wall 1914. The tapered side wall 1914 may be formed as part of the distal region 1910. The tapered side wall 1914 may include a taper angle θ of 0.0 degrees to 30 degrees. In some examples, taper angle θ is 10 degrees to 15 degrees. The body of nozzle 1900 may be tapered at just the distal region 1910, as shown in FIGS. 19A-19D . In some examples, the proximal region of the nozzle may be tapered as well. In some examples, the proximal region of the nozzle may be tapered less than the distal region. In some examples, the proximal region 1920 includes a cylindrical portion 1916. The cylindrical portion 1916 may have a longitudinal length of at least 3 millimeters (mm). In some embodiments, the cylindrical portion 1916 may have a length of at least 5 millimeters (mm). In some embodiments, the cylindrical portion 1916 includes a length that is at least 30 percent of the overall longitudinal length of nozzle 1900. By longitudinally spacing the tapered side wall from shock wave emitters, sonic output may be increased.

A wall thickness (i.e., a difference between outer diameter and inner diameter) of the body of nozzle 1900 may be at least 0.50 millimeters (mm). In some examples, the wall thickness is at least 0.75 millimeters (mm). In some examples, the wall thickness is at least 1.0 millimeters (mm). In some examples, the wall thickness is constant from the distal region 1910 to the proximal region 1920. In some examples, the wall thickness at the distal region is larger than the wall thickness at the proximal region. In some examples, the nozzle 1900 includes a transition region 1918 (e.g., at the bent region between the tapered side wall 1914 and cylindrical portion 1916). The wall thickness at the transition region 1918 of the nozzle body may be larger than at the distal region 1910 to provide structural support to the nozzle at the bent region of the nozzle.

Nozzle 1900 may have a longitudinal length no less than 5.0 millimeters (mm). In some examples, the longitudinal length is no less than 10 millimeters (mm). In embodiments where the shock wave emitters (e.g., spark gaps) are axially aligned with the proximal outlet 1922, the longitudinal length may be the same as the distance of the emitters from the distal outlet 1912. In some examples, the nozzle outlet 220 has a diameter of 3 millimeters (mm) to 15 millimeters (mm). In some examples, the nozzle outlet 220 has a diameter of at least 5 millimeters (mm). In some examples, the nozzle outlet 220 has a diameter of less than 10 millimeters (mm).

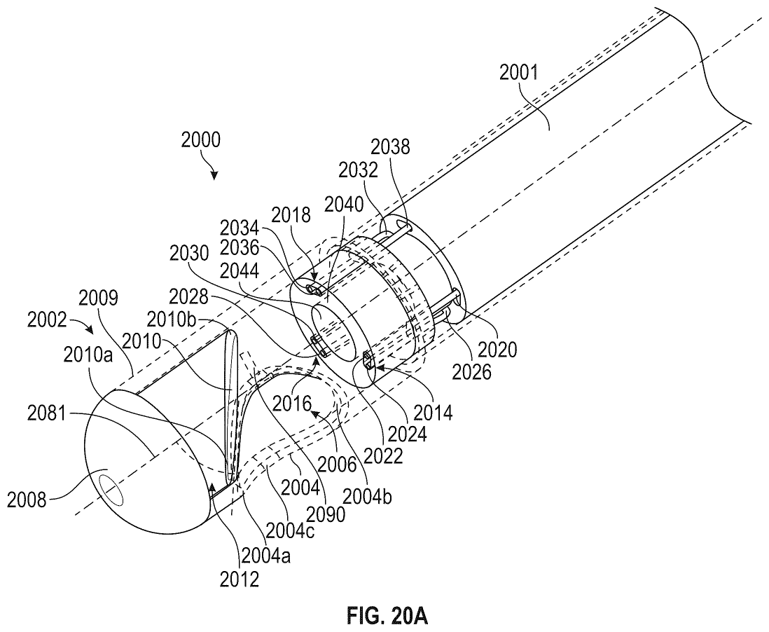

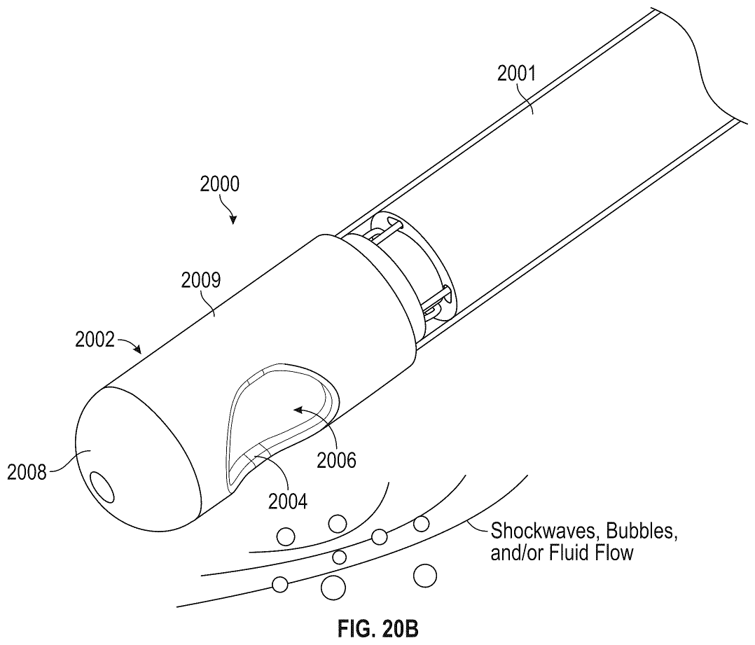

As discussed with reference to FIG. 4B, it may be desirable in some examples to direct shock wave energy at least partially radially outward. FIGS. 20A-20B illustrate aspects of an exemplary catheter 2000, which can be used for catheter 10 of FIG. 1, that can direct shock wave energy at least partially radially outward. Catheter 2000 includes a catheter body 2001 having a distal end 2040. A plurality of shock wave emitters 2014, 2016, and 2018 are positioned at the distal end 2040 of catheter body 2001. The plurality of shock wave emitters 2014, 2016, and 2018 may include any of the aspects described with respect to the shock wave emitters of any of the examples disclosed herein. In some examples, the plurality of shock wave emitters 2014, 2016, and 2018 include aspects of the shock wave emitters disclosed with reference to FIGS. 2 and/or 8A-8C. A cap 2002 of catheter 2000 is positioned at least partially distally of the plurality of shock wave emitters 2014, 2016, and 2018 and is configured to redirect shock waves and/or cavitation bubbles at least partially radially outward toward a lesion. A distal end 2008 of the cap 2002 may be enclosed and the cap 2002 may include an internal deflector 2010 configured to redirect shock waves and/or cavitation bubbles generated using the plurality of shock wave emitters 2014, 2016, and 2018 out of an aperture 2004 formed into a cylindrical wall 2009 of the cap 2002.

The cap 2002 may include a cavity 2006 between the plurality of shock wave emitters 2014, 2016, and 2018 and the deflector 2010. Catheter 2000 may be configured to introduce a conductive fluid into the cavity 2006, which may flow out of the aperture 2004 toward a lesion during shock wave generation. The catheter body 2001 may include a fluid lumen, such as lumen 2044, configured to replenish conductive fluid into the cavity 2006 to replace the fluid lost during shock wave generation. In some examples, a distal edge 2010a of the deflector 2010 may be longitudinally aligned with a distal edge 2004a of the aperture 2004. A proximal edge 2010b of the deflector 2010 may be longitudinally aligned with a proximal edge 2004b of the aperture 2004. Aligning the deflector 2010 with aperture 2004 may ensure a maximal sonic output is reflected through the aperture 2004 toward a lesion. In some examples, the deflector 2010 may be formed at an angle 2012 relative to a longitudinal axis 2081. The angle 2012 between the deflector 2010 and longitudinal axis 2081 may be between 0 degrees and 90 degrees. In some examples, the angle 2012 between the deflector 2010 and the longitudinal axis 2081 is an oblique angle.

In some examples, an inner diameter of the cap 2002 is larger than an outer diameter of the catheter body 2001. The cap 2002 may be removably attached to the catheter body 2001, permanently affixed to the catheter body 2001 or may be integrally formed with the catheter body 2001. The deflector 2010 may be flat, curved in a convex manner, and/or curved in a concave manner. An edge 2004c defining the aperture 2004 may be filleted to promote fluid flow outward from the cavity 2006 through the aperture 2004. In some examples, the aperture 2004 is covered by a membrane configured to allow shock waves to pass through the aperture 2004 while inhibiting fluid and/or particulate flow through the aperture 2004. The deflector 2010 may be formed from or include an acoustically reflective material configured to reflect shock waves. For instance, the deflector may be formed from or include a metallic material. In some examples, a radiopaque marker 2090 is positioned on the cap 2002 to enable a user to align the aperture 2004 with a treatment site. In some examples multiple radiopaque markers 2090, optionally having different shapes or sizes are positioned on the cap 2002 to enable a user to determine a circumferential location of the aperture 2004 relative to a target treatment site. For instance, radiopaque markers 2004 of different shapes or sizes may be positioned incrementally (e.g., at 10-degree offsets) around a circumference of the outer wall 2009, which may be a cylindrical outer wall. A user may determine the orientation of the aperture 2004 based on the type or shape of radiopaque marker 2090 visible via an imager positioned, for instance, above a patient during a procedure. In some examples, one or more radiopaque markers 2090 may be aligned with an edge (e.g., 2004c) of the aperture 2004. For instance, a first radiopaque marker 2090 may be aligned with the distal edge 2004a of aperture 2004 and a second radiopaque marker 2090 may be aligned with a proximal edge 2004b of the aperture 2004.

In the exemplary embodiment illustrated in FIG. 20A, a first shock wave emitter 2014 includes an exposed (conductive) distal tip 2022 of a first insulated wire 2020 and an exposed distal tip 2024 of a second insulated wire 2026 separated by a spark gap. As discussed above, an “exposed end,” “exposed tip,” and/or “exposed distal tip” of an insulated wire may refer to a portion of the wire from which the insulation has been removed, thus revealing a portion of the conductive wire. However, while the emitters herein are often described as including the exposed distal ends/tips of insulated wires, it should be understood that any suitable conductor may serve as an electrode of the emitters. The insulated wire 2020 extends along the length of the catheter body 2001 from the distal end 2040 (e.g., so that it can be connected to a voltage source proximally of the distal end, for instance, at a proximal end of the catheter). The second insulated wire 2026 extends from the first shock wave emitter 2014 to the second shock wave emitter 2016. The second insulated wire 2026 includes another exposed distal tip 2028 forming part of an electrode pair at shock wave emitter 2016.

The second insulated wire 2026 extends proximally from shock wave emitter 2014 into the catheter body 2001 for a first distance, and loops around (for instance as discussed with respect to the bend 895 forming the U-shaped portion of insulated wire 814 shown in FIG. 8A), and then extends distally toward shock wave emitter 2016. The electrode pair at shock wave emitter 2016 includes an exposed distal tip 2030 of a third insulated wire 2032 separated from the distal tip 2028 of insulated wire 2026 by a spark gap. The third insulated wire extends from the second shock wave emitter 2016 to a third shock wave emitter 2018 and includes another exposed distal tip 2034 forming part of an electrode pair at shock wave emitter 2018. The third insulated wire 2032 extends proximally from shock wave emitter 2016 into the catheter body 2001 for a first distance, loops around, and then extends distally toward shock wave emitter 2018. The electrode pair at shock wave emitter 2018 includes an exposed distal tip 2036 of a fourth insulated wire 2038 separated from the distal tip 2034 of wire 2032. The fourth insulated wire may extend along the length of the catheter body 2001 from the distal end 2040 (e.g., so that it can be connected to a voltage source proximally of the distal end, for instance, at a proximal end of the catheter).

During use of catheter 2000, a voltage pulse may be applied across wires 2020 and 2038, which may cause an electrical current to jump across the respective spark gaps at each of shock wave emitters 2014-2018, resulting in at least one respective shock wave and/or cavitation bubble being generated by the shock wave emitters 2014-2018. The shock wave(s) and/or cavitation bubble(s) may travel distally from the shock wave emitters 2014-2018 toward the deflector 2010 within cavity 2006. The shock wave(s) and/or cavitation bubble(s) may additionally induce a fluid flow toward the deflector 2010. The deflector 2010 may redirect the shock wave(s), cavitation bubble(s), and/or fluid flow at least partially radially outward through the aperture 2004 and away from the catheter body 2001 toward a lesion or other target treatment site. A combined force imparted by the shock wave(s), cavitation bubble(s), and fluid flow may be greater than that provided by conventional radially firing shock wave emitters, which are typically enclosed within a balloon that prevents direct impact of cavitation bubbles and/or fluid flow on lesions within the body. Thus, the cap 2002 and its deflector 2010 may enable enhanced treatment of different lesions within the body. Additionally, a user may rotate catheter 2000 to position aperture 2004 such that it is adjacent to different circumferential locations of a lesion, enabling a user to effectively cover a 360-degree treatment region.

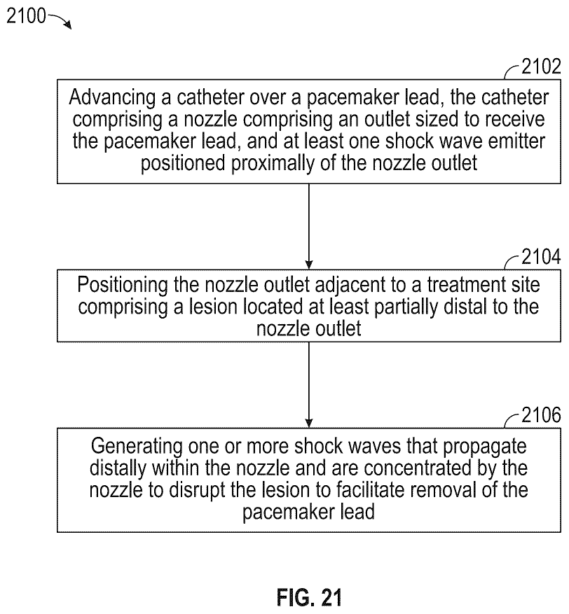

FIG. 21 illustrates an exemplary method 2100 of facilitating pacemaker lead removal using a shock wave catheter. Method 2100 may be performed using aspects of the catheters disclosed herein, including, for instance, catheter 10, catheter 200, catheter 400, catheter 500, catheter 800, catheters 900a-900d, catheter 1000, catheter 1100, catheter 1200, catheter 1300, and/or catheter 1820. At block 2102, method 2100 may include advancing a catheter over a pacemaker lead. The catheter may include a nozzle that includes an outlet sized to receive the pacemaker lead, and at least one shock wave emitter positioned proximally of the nozzle outlet. The nozzle may be a convergent nozzle configured to concentrate the one or more shock waves at or beyond the nozzle outlet. In some examples, the at least one shock wave emitter includes a plurality of shock wave emitters arrayed symmetrically about a longitudinal axis of the catheter. The at least one shock wave emitter may be formed by a distal end of a first wire and a distal end of a second wire separated by a spark gap.

At block 2104, method 2100 may include positioning the nozzle outlet adjacent to a treatment site comprising a lesion located at least partially distal to the nozzle outlet. The treatment site may be within the heart. The lesion may include scarred tissue, calcified tissue, fibrotic tissue, and/or multi-morphic tissue. The lesion may be disposed at least partially on the pacemaker lead. Thus, as the catheter is advanced over the pacemaker lead, the lesion may be received into the nozzle outlet. The catheter may be advanced along the pacemaker lead such that the pacemaker lead is inserted into a lumen of the catheter proximal to the nozzle. Thus, the pacemaker lead may effectively act as a guidewire for the catheter.

At block 2106, method 2100 may include generating one or more shock waves that propagate distally within the nozzle and are concentrated by the nozzle to disrupt the lesion to facilitate removal of the pacemaker lead. The shock waves concentrated at the nozzle outlet may impinge on the lesion within the nozzle and at the outlet. The nozzle may be configured such that a substantially uniform pressure (e.g., a uniform circumferential pressure) is applied to the pacemaker lead by the one or more shock waves. The one or more shock waves may impinge upon the lesion distal of the catheter. In some examples, the method includes directing, by the nozzle, a plurality of bubbles generated by the at least one shock wave emitter toward an outlet of the nozzle. Directing the plurality of bubbles toward the outlet of the nozzle may cause the plurality of bubbles to concentrate at the outlet and/or propagate distally of the outlet.

In some examples, the nozzle outlet may include a beveled edge or tapered edge. The method may include advancing the catheter distally toward the lesion and scraping the lesion from the pacemaker lead using the beveled edge. In some examples, the method may include supplying a fluid to an inlet of the nozzle while the one or more shock waves are generated. In some examples, the method may include aspirating debris from a body lumen in which the pacemaker lead is positioned via the outlet of the nozzle. The nozzle may be formed from an acoustically reflective material. In some examples, the nozzle may be configured to concentrate the one or more shock waves at a longitudinal axis of the catheter. In some examples, the nozzle may be configured to concentrate the one or more shock waves at an position offset from a longitudinal axis of the catheter.

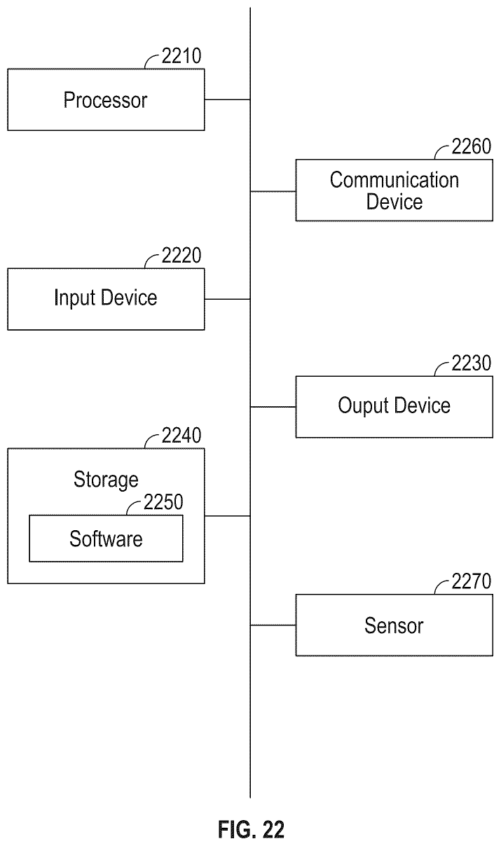

FIG. 22 illustrates an example of a computing system 2200 that can be used for one or more components of the system for catheter 100 of FIG. 1, such as subsystem 150 for controlling energy delivery to catheter 100. System 2200 can be a computer connected to a network, such as one or more networks of hospital, including a local area network within a room of a medical facility and a network linking different portions of the medical facility, or a wide-area network accessed through the internet or other means. System 2200 can be a client or a server. System 2200 can be any suitable type of processor-based system, such as a personal computer, workstation, server, handheld computing device (portable electronic device), such as a phone or tablet, or dedicated device. System 2200 can include, for example, one or more of input device 2220, output device 2230, one or more processors 2210, storage 2240, and communication device 2260. Input device 2220 and output device 2230 can generally correspond to those described above and can either be connectable or integrated with the computer.

Input device 2220 can be any suitable device that provides input, such as a touch screen, keyboard or keypad, mouse, gesture recognition component of a virtual/augmented reality system, or voice-recognition device. Output device 2230 can be or include any suitable device that provides output, such as a display, touch screen, haptics device, virtual/augmented reality display, or speaker.

Storage 2240 can be any suitable device that provides storage, such as an electrical, magnetic, or optical memory including a RAM, cache, hard drive, removable storage disk, or other non-transitory computer-readable medium. Communication device 2260 can include any suitable device capable of transmitting and receiving signals over a network, such as a network interface chip or device. The components of the computing system 2200 can be connected in any suitable manner, such as via a physical bus or wirelessly.

Processor(s) 2210 can be any suitable processor or combination of processors, including any of, or any combination of, a central processing unit (CPU), field programmable gate array (FPGA), and application-specific integrated circuit (ASIC). Software 2250, which can be stored in storage 2240 and executed by one or more processors 2210, can include, for example, the programming that embodies the functionality or portions of the functionality of the present disclosure (e.g., as embodied in the devices as described above), such as programming for performing one or more steps of method 200, method 300, and/or method 600.

Software 2250 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 2240, that can contain or store programming for use by or in connection with an instruction execution system, apparatus, or device.

Software 2250 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by or in connection with an instruction execution system, apparatus, or device. The transport computer-readable medium can include, but is not limited to, an electronic, magnetic, optical, electromagnetic, or infrared wired or wireless propagation medium.

System 2200 may include a sensor device 2270 that provides sensor data for processing by processor 2210. Sensor device 2270, in some embodiments, may be an imaging sensor that provides imaging data, for a lesion being treated. In some embodiments, sensor device 2270 may be a voltage sensor, a current sensor, a pressure sensor, a temperature sensor, or an optical sensor for providing data about a state of the catheter or a lesion.

System 2200 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

System 2200 can implement any operating system suitable for operating on the network. Software 2250 can be written in any suitable programming language, such as C, C++, Java, or Python. In various examples, application software embodying the functionality of the present disclosure can be deployed in different configurations, such as in a client/server arrangement or through a Web browser as a Web-based application or Web service.

System 2200 may be configured to selectively control the delivery of energy from one or more of energy sources (e.g., a voltage pulse generator or a light energy source) to one or more acoustic energy emitters (e.g., a forward-firing emitter, a radially-firing emitter, an unenclosed emitter, or an enclosed emitter) depending on input from input device 2220.

System 2200 may be configured to tune the energy properties of energy delivered to one or more of the above-described emitters based on tissue properties received from sensor device 2270. Tissue properties may include lesion tissue type (e.g., calcific, thrombic, fibrotic, etc.), lesion morphology (e.g., thickness, length, eccentricity, density, stiffness, etc.).

According to aspects of the disclosure, a method of refurbishing a shock wave catheter may include replacing or repairing one or more components of the catheter, such as a nozzle, electrode, catheter body, wiring, and so on. For example, a nozzle may be replaced by detaching the nozzle from a distal end of a catheter attaching a new nozzle to the distal end of the catheter. This may include screwing/unscrewing the nozzle from the catheter, prying the nozzle from the catheter, cutting the nozzle from the catheter, etc. In some examples, the nozzle may be refurbished either in place or after having been removed from the catheter. Refurbishment of the nozzle may include, for example, unclogging the nozzle and/or sharpening a beveled edge of the nozzle. Refurbishing of a catheter may include replacing one or more components of a shock wave emitter assembly. This can include replacing one or more wires or removing a portion of one or more wires and soldering a new wire to the remaining portion and/or replacing one or more emitter sheaths. Optionally, an entire electrode assembly is removed from the catheter, repaired, and reassembled to the catheter. Optionally, an entire electrode assembly may be removed and replaced. Optionally, refurbishing a shock wave emitter assembly may include testing one or more performance parameters of a refurbished shock wave emitter. Testing may include testing the ability of the nozzle to concentrate shock waves and/or cavitation bubble, such as by generating one or more shock waves and measuring a sonic output of the catheter. Testing may include testing the shock wave emitter assembly, such as by applying one or more voltage pulses to the shock wave emitter assembly and observing whether sparks are formed and/or measuring an intensity of the resulting shock waves.

Although the electrode assemblies and catheter devices described herein have been discussed primarily in the context of treating coronary occlusions, such as lesions in vasculature, the electrode assemblies and catheters herein can be used for a variety of occlusions, such as occlusions in the peripheral vasculature (e.g., above-the-knee, below-the-knee, iliac, carotid, etc.). For further examples, similar designs may be used for treating soft tissues, such as cancer and tumors (i.e., non-thermal ablation methods), blood clots, fibroids, cysts, organs, multi-morphology, scar, and fibrotic tissue removal, or other tissue destruction and removal. Electrode assembly and catheter designs could also be used for neurostimulation treatments, targeted drug delivery, treatments of tumors in body lumens (e.g., tumors in blood vessels, the esophagus, intestines, stomach, or vagina), wound treatment, non-surgical removal and destruction of tissue, or used in place of thermal treatments or cauterization for venous insufficiency and fallopian ligation (i.e., for permanent female contraception).

In one or more examples, the electrode assemblies and catheters described herein could also be used for tissue engineering methods, for instance, for mechanical tissue decellularization to create a bioactive scaffold in which new cells (e.g., exogenous or endogenous cells) can replace the old cells; introducing porosity to a site to improve cellular retention, cellular infiltration/migration, and diffusion of nutrients and signaling molecules to promote angiogenesis, cellular proliferation, and tissue regeneration similar to cell replacement therapy. Such tissue engineering methods may be useful for treating ischemic heart disease, fibrotic liver, fibrotic bowel, and traumatic spinal cord injury (SCI). For instance, for the treatment of spinal cord injury, the devices and assemblies described herein could facilitate the removal of scarred spinal cord tissue, which acts like a barrier for neuronal reconnection, before the injection of an anti-inflammatory hydrogel loaded with lentivirus to genetically engineer the spinal cord neurons to regenerate.

It should be understood that the foregoing is only illustrative of the principles of the invention, and that various modifications, alterations and combinations can be made by those skilled in the art without departing from the scope and spirit of the invention. Any of the variations of the various catheters disclosed herein can include features described by any other catheters or combination of catheters herein. Furthermore, any of the methods can be used with any of the catheters disclosed. Accordingly, it is not intended that the invention be limited, except as by the appended claims.

1. A method of facilitating pacemaker lead removal using a shock wave catheter, the method comprising:
advancing a catheter over a pacemaker lead, the catheter comprising a nozzle comprising an outlet sized to receive the pacemaker lead, and at least one shock wave emitter positioned proximally of the nozzle outlet;
positioning the nozzle outlet adjacent to a treatment site comprising a lesion located at least partially distal to the nozzle outlet;
generating one or more shock waves that propagate distally within the nozzle and are concentrated by the nozzle to disrupt the lesion to facilitate removal of the pacemaker lead.

2. The method of claim 1, wherein the treatment site is within the heart.

3. The method of claim 1, wherein the nozzle is a convergent nozzle configured to concentrate the one or more shock waves at or beyond the nozzle outlet.

4. The method of claim 1, wherein the lesion is disposed at least partially on the pacemaker lead such that as the catheter is advanced over the pacemaker lead, the lesion is received into the nozzle outlet.

5. The method of claim 4, wherein the shock waves concentrated at the nozzle outlet impinge on the lesion within the nozzle and at the outlet.

6. The method of claim 1, wherein the nozzle is configured such that a substantially uniform pressure is applied to the pacemaker lead by the one or more shock waves.

7. The method of claim 1, wherein the nozzle outlet comprises a beveled edge or a tapered edge.

8. The method of claim 7, further comprising advancing the catheter distally toward the lesion and scraping the lesion from the pacemaker lead using the beveled edge.

9. The method of claim 1, wherein the one or more shock waves impinge upon the lesion distally of the nozzle outlet.

10. The method of claim 1, comprising supplying a fluid to an inlet of the nozzle while the one or more shock waves are generated.

11. The method of claim 1, comprising: directing, by the nozzle, a plurality of bubbles generated by the at least one shock wave emitter toward an outlet of the nozzle, wherein the nozzle concentrates the bubbles at the outlet.

12. The method of claim 1, comprising, aspirating debris from a body lumen in which the pacemaker lead is positioned via the outlet of the nozzle.

13. The method of claim 1, wherein the nozzle is formed from an acoustically reflective material.

14. The method of claim 1, wherein the nozzle is configured to concentrate the one or more shock waves at a longitudinal axis of the catheter.

15. The method of claim 1, wherein the nozzle is configured to concentrate the one or more shock waves at an position offset from a longitudinal axis of the catheter.

16. The method of claim 1, wherein the at least one shock wave emitter comprises a plurality of shock wave emitters arrayed symmetrically about a longitudinal axis of the catheter.

17. The method of claim 1, wherein the at least one shock wave emitter is formed by a distal end of a first wire and a distal end of a second wire separated by a spark gap.

18. A catheter for use in a body lumen, the catheter comprising:
a catheter body;
at least one shock wave emitter disposed at a distal end of the catheter body and configured to generate at least one shock wave that propagates distally of the catheter body; and
a nozzle disposed at a distal end of the catheter body and configured to direct the at least one shock wave to an outlet of the nozzle.

19. A method for treating an occlusion in a body lumen with shock waves, the method comprising:
positioning a distal portion of a catheter adjacent to the occlusion in the body lumen;
emitting one or more shock waves from one or more shock wave emitters located at the distal portion of the catheter such that the shock waves propagate in a distal direction; and
directing the shock waves by a nozzle located at a distal end of the catheter to an outlet of the nozzle for treating the occlusion.

20. A catheter for treating a lesion in a body lumen, the catheter comprising:
a catheter body;
at least one shock wave emitter disposed at a distal end of the catheter body and configured to generate at least one shock wave;
a cap positioned at least partially distally of the at least one shock wave emitter, the distal cap comprising:
a closed distal end;
an outer wall extending between the catheter body and the distal end, the outer wall comprising an aperture; and
a deflector positioned between the distal end and the at least one shock wave emitter, the deflector oriented at an oblique angle relative to a longitudinal axis of the catheter body and configured to direct shock waves generated using the at least one shock wave emitter outward through the aperture.
